(19) United States
(12) Patent Application Publication
(10) Pub. No.: US 2005/0096327 A1
(43) Pub. Date: May 5, 2005

(54) Novel norepinephrine reuptake inhibitors for the treatment of central nervous system disorders

(76) Inventors: Bradley William Caprathe, Livonia, MI (US); Shelly Ann Glase, Ann Arbor, MI (US); Zissis Konstantinou, Scarborough (CA); Robert Michael Schelkun, Milan, MI (US); Susan M. Sheehan, Dexter, MI (US); Anthony Jerome Thomas, Ann Arbor, MI (US); Po-Wai Yuen, Ann Arbor, MI (US)

Correspondence Address: WARNER-LAMBERT COMPANY, 2800 PLYMOUTH RD, ANN ARBOR, MI, 48105, US

(21) Appl. No.: 10/979,651

(22) Filed: Nov. 2, 2004

Related U.S. Application Data
(60) Provisional application No. 60/516,879, filed on Nov. 3, 2003.
(60) Provisional application No. 60/611,292, filed on Sep. 21, 2004.

(51) Int. Cl. A61K 31/517; C07D 43/02

(52) U.S. Cl. 514/252.17; 514/266.22; 544/284

(57) ABSTRACT

This invention relates to compounds of the formula 1 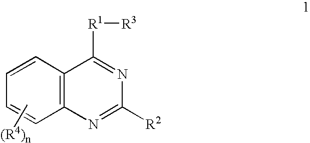
wherein R¹, R², and R³ and R⁴ are defined as in the specification, pharmaceutical compositions containing them and their use in the treatment of central nervous system disorders.

This invention relates to a method of preventing or treating attention deficit hyperactivity disorder (“ADHD”) by administering a compound that inhibits the reuptake of norepinephrine. Such compounds are also referred to in the literature as selective norepinephrine reuptake inhibitors (NRIs).

### BACKGROUND OF THE INVENTION

Attention deficit hyperactivity disorder (ADHD) has an estimated incidence in school age children of 3-5%, and is characterized by the core symptoms of hyperactivity, impulsivity, and/or inattention. The attentional symptoms of ADHD can be successfully treated with psychomotor stimulants such as methylphenidate (Ritalin). Clonidine, an α₂-adrenoceptor agonist, treats the aggressive and oppositional symptoms. There is a potential for significant side effects with both methylphenidate and clonidine, making it important to identify other drugs that have similar or better efficacy with reduced side effects and abuse liability.

ADHD is one of the most common childhood psychiatric disorders and appears to be a common, often underrecognized, psychiatric disease in adults as well (T. Spencer, et al., J Clin Psychiatry, 1998, 59(Suppl. 7), 759-768). This disorder, which begins in childhood, may be followed by a lifelong expression of symptoms (e.g., inattention and/or impulsivity) (J B. Schweitzer, et al., Med Clin North Am, May 2001, 85:3, 757-777). ADHD may change its manifestations as it develops from preschool through adult life (D P. Cantwell, J Am Acad Child Adolesc Psychiatry, Aug. 1996, 35(8), 978-987; J. Elia, et al. N Eng J Med, Mar. 1999, 340(10), 780-788; E E. Nolan, et al., J Am Acad Child Adolesc Psychaitry, Feb. 2001, 40(2), 241-249).

The diagnosis of ADHD is based on clinical evaluation (M. Dulcan, et al. M,J Am Acad Child Adolesc Psychaitry, Oct. 1997, 36(10 Suppl), 85S-121S; National Institutes of Health, 1998). “The essential feature of ADHD is a persistent pattern of inattention and/or hyperactivity-impulsivity that is more frequent and severe than is typically observed in individuals at a comparative level of development” (Diagnostic and Statistical Manual of Mental Disorders (DSM-IV), American Psychiatric Association, Washington, D.C., 1994). In order to be diagnosed with ADHD, patients must demonstrate symptoms of ADHD that cause impairment before the age of seven years, and symptoms must have been ongoing for longer than six months in at least two settings (e.g., school [or work] and home). (See DSM-IV).

Several NRI compounds are known. Atomoxetine, an NRI, is now commercially available (Strattera®, Eli Lilly) and is beginning to be used extensively for the clinical treatment of ADHD in both children and adults. Atomoxetine represents a non-stimulant treatment for ADHD. The number of treated ADHD patients is expected to increase as a result of the introduction of atomoxetine and enhanced educational initiatives. Accordingly, there is an ongoing need for ADHD treatments that provide more efficacy than those treatments currently available.

### SUMMARY OF THE INVENTION

The present invention relates to compounds of the formula 1 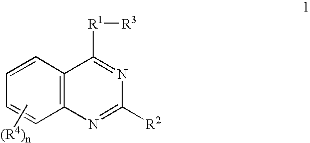
or a pharmaceutically acceptable salt, solvate or hydrate thereof, wherein
R¹ is (C₁-C₆)alkyl, (C₃-C₈)cycloalkyl, (C₁-C₆)alkoxy, aryl, amino, halogen, hydroxy, heteroaryl, or a saturated, unsaturated, or aromatic five to seven membered monocyclic heterocyclic ring containing from one to three heteroatoms independently selected from oxygen, nitrogen, and sulfur;
R² is (C₁-C₆)alkyl, (C₃-C₈)cycloalkyl, amino, or a saturated, unsaturated, or aromatic five to seven membered monocyclic heterocyclic ring or a six to ten membered bicyclic ring containing from one to three heteroatoms independently selected from oxygen, nitrogen, and sulfur, any of which may be unsubstituted or substituted with one or more of the following substituents: (C₁-C₆)alkyl, substituted (C₁-C₆)alkyl, amino, (C₁-C₆)alkylamino, or a heterocyclic group;
R³ is independently selected from one or more of hydrogen, (C₁-C₆)alkyl, (C₃-C₈)cycloalkyl, (C₁-C₆)alkoxy, aryl, amino, halogen, or hydroxy groups;
R⁴ is independently selected from one or more of hydrogen, halogen, —NO₂, (C₁-C₆)alkyl, (C₁-C₆)alkoxy, or a heterocyclic group, wherein each occurrence of R⁴ may be the same or different; and n is 0, 1, 2, or 3.

A preferred embodiment of this invention relates to compounds of the formula 1 wherein R¹ is aryl and R² is piperazinyl or piperidinyl.

A further preferred embodiment of the invention relates to compounds wherein R¹ is a phenyl group. More preferred embodiments relate to compounds wherein R¹ is a substituted phenyl group. Yet a further preferred embodiment of the invention relates to compounds wherein R¹ is a halogen substituted phenyl group.

A further preferred embodiment of the invention relates to compounds of the formula 1B 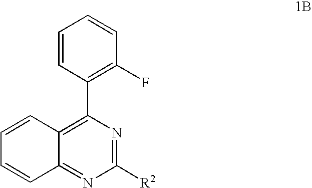
wherein R² is substituted or unsubstituted piperidinyl, piperazinyl, homopiperazinyl, or 3-aminopyrrolidinyl, and the pharmaceutically acceptable salts thereof.

A further preferred embodiment of the invention relates to compounds of the formula 1B wherein R² is

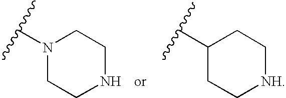

Preferred compounds of the invention include the following compounds and their pharmaceutically acceptable salts, solvates, and hydrates:
2-(4-Methyl-piperazin-1-yl)-4-phenyl-quinazoline;
2-(4-Methyl-piperazin-1-yl)-4-p-tolyl-quinazoline;
4-Phenyl-2-piperazin-1-yl-quinazoline;
2-(4-Methyl-piperazin-1-yl)-4-o-tolyl-quinazoline;
2-(3-Methyl-3,9-diaza-bicyclo[3.3.1]non-9-yl)-4-phenyl-quinazoline;
4-Isopropyl-2-piperazin-1-yl-quinazoline;
2-[1,4]Diazepan-1-yl-4-phenyl-quinazoline;
2-[1,4]Diazepan-1-yl-4-isopropyl-quinazoline;
2-(2,5-Dimethyl-piperazin-1-yl)-4-phenyl-quinazoline;
2-(2,5-Diaza-bicyclo[2.2.1]hept-2-yl)-4-phenyl-quinazoline;
2-[1-(4-Phenyl-quinazolin-2-yl)-piperidin-3-yl]-ethylamine;
1-(4-Phenyl-quinazolin-2-yl)-piperidin-4-ylamine;
N¹-(4-Phenyl-quinazolin-2-yl)-ethane-1,2-diamine;
1-(4-Phenyl-quinazolin-2-yl)-pyrrolidin-3-ylamine;
2-(2-Methyl-piperazin-1-yl)-4-phenyl-quinazoline;
1-(4-Phenyl-quinazolin-2-yl)-pyrrolidin-3-yl amine;
3-(4-Phenyl-quinazolin-2-yl)-aminopyrrolidine;
4-(2-Fluoro-phenyl)-2-(4-methyl-piperazin-1-yl)-quinazoline;
4-(2-Chloro-phenyl)-2-(4-methyl-piperazin-1-yl)-quinazoline;
4-(2-Fluoro-phenyl)-2-piperazin-1-yl-quinazoline;
2-[1,4]Diazepan-1-yl-4-(2-fluoro-phenyl)-quinazoline;
4-(2-Chloro-phenyl)-2-piperazin-1-yl-quinazoline;
4-(2-Methoxy-phenyl)-2-piperazin-1-yl-quinazoline;
4-(2-Methyl-phenyl-2-piperazin-1-yl)quinazoline;
4-(4-Fluoro-phenyl)-2-(4-methyl-piperazin-1-yl)-quinazoline;
4-(3-Fluoro-phenyl)-2-(4-methyl-piperazin-1-yl)-quinazoline;
2-(4-Methyl-piperazin-1-yl)-4-thiophen-2-yl-quinazoline;
4-Benzyl-2-piperazin-1-yl-quinazoline;
4-(2,6-Difluoro-phenyl)-2-piperazin-4-yl-quinazoline;
(R)-(−)-2-(2-Methyl-piperazin-1-yl)-4-phenyl-quinazoline;
(R)-(+)-2-(3-Methyl-piperazin-1-yl)-4-phenyl-quinazoline;
2-(3,9-Diaza-bicyclo[3.3.1]non-3-yl)-4-phenyl-quinazoline;
2-(3,9-Diaza-bicyclo[3.3.1]non-3-yl)-4-(2-fluoro-phenyl)-quinazoline;
(S)-(+)-1-[4-(2-Fluoro-phenyl)-quinazolin-2-yl]-pyrrolidin-3-ylamine;
(S)-(+)-{1-[4-(2-Fluoro-phenyl)-quinazolin-2-yl]-pyrrolidin-3-yl}-methyl-amine;
4-(2-Chloro-6-fluoro-phenyl)-2-piperazin-1-yl-quinazoline;
4-(2,3-Difluoro-phenyl)-2-piperazin-1-yl-quinazoline;
4-(2,4-Difluoro-phenyl)-2-piperazin-1-yl-quinazoline;
4-(2-Fluoro-phenyl)-2-(hexahydro-pyrrolo[3,4-c]pyrrol-2-yl)-quinazoline;
(S)-(+)-1-[4-(2-Fluoro-phenyl)-quinazolin-2-yl]-piperidin-3-ylamine;
4-(2-Fluoro-phenyl)-2-(piperidin-4-yl)-quinazoline;
4-Phenyl-2-piperidin-4-yl-quinazoline;
4-(2-Fluoro-phenyl)-2-(1-methyl-piperidin-4-yl)-quinazoline;
4-(2-Chloro-phenyl)-2-(1-methyl-piperidin-4-yl)-quinazoline;
4-(2-Chloro-phenyl)-2-piperidin-4-yl-quinazoline;
4-(2-Methoxy-phenyl)-2-piperidin-4-yl-quinazoline;
4-(2-Methyl-phenyl)-2-piperidin-4-yl-quinazoline;
4-Phenyl-2-piperidin-3-yl-quinazoline;
4-(4-Phenyl-quinazolin-2-yl)piperidine-4-carboxylic acid methyl ester;
4-[4-(2-Fluoro-phenyl)-quinazolin-2-yl]-piperidine-4-carboxylic acid methyl ester;
3-(4-Phenyl-quinazolin-2-yl)-piperidine-3-carboxylic acid methyl ester;
2-Piperazin-1-yl-4-s-tolyl-quinazoline;
2-(3-Methyl-piperazin-1-yl)-4-phenyl-quinazoline;
2-(3,9-Diaza-bicyclo[3.3.1]non-9-yl)-4-phenyl-quinazoline;
2-(3,8-Diaza-bicyclo[3.2.1]oct-8-yl)-4-phenyl-quinazoline;
2-[1,4]Diazepan-1-yl-4-(2,3-difluoro-phenyl)-quinazoline;
4-(2,6-Difluoro-phenyl)-quinazolin-2-yl]-pyrrolidin-3-yl-amine;
7-Fluoro-4-(2-fluoro-phenyl)-2-piperazin-1-yl-quinazoline;
4-(3-Fluoro-phenyl)-2-piperazin-1-yl-quinazoline;
4-(2-Chloro-4-fluoro-phenyl)-2-piperazin-1-yl-quinazoline;
4-(4-Chloro-phenyl)-2-piperazin-1-yl-quinazoline;
4-(2,6-Dichloro-phenyl)-2-piperazin-1-yl-quinazoline;
6-Fluoro-4-(2-fluoro-phenyl)-2-piperidin-4-yl-quinazoline;
7-Fluoro-4-(2-fluoro-phenyl)-2-piperidin-4-yl-quinazoline;
4-(3-Fluoro-phenyl)-2-piperidin-4-yl-quinazoline;
4-(3-Fluoro-phenyl)-2-(1-methyl-piperidin-4-yl)-quinazoline;
4-(4-Fluoro-phenyl)-2-piperidin-4-yl-quinazoline;
4-(2,6-Difluoro-phenyl)-2-(1-methyl-piperidin-4-yl)-quinazoline;
4-(2,6-Difluoro-phenyl)-2-piperidin-4-yl-quinazoline;
4-(2,3-Difluoro-phenyl)-2-piperidin-4-yl-quinazoline;
4-(2,4-Difluoro-phenyl)-2-piperidin-4-yl-quinazoline;
2-Piperidin-4-yl-4-(2,3,6-trifluoro-phenyl)-quinazoline;
4-(2-Chloro-6-fluoro-phenyl)-2-(1-methyl-piperidin-4-yl)-quinazoline;
4-(2-Chloro-6-fluoro-phenyl)-2-piperidin-4-yl-quinazoline;
2-Piperidin-4-yl-4-o-tolyl-quinazoline;
4-(2-Fluoro-phenyl)-2-piperidin-3-yl-quinazoline;
2-Piperidin-3-yl-4-o-tolyl-quinazoline;
4-(2-Fluoro-phenyl)-2-(4-phenyl-piperidin-4-yl)-quinazoline;
2-(2,5-Diaza-bicyclo[2.2.1]hept-2-yl)-4-phenyl-quinazoline;
2-(Hexahydro-pyrrolo[3,4-c]pyrrol-2-yl)-4-phenyl-quinazoline;
4-(2,4-Difluoro-phenyl)-2-(2-methyl-piperazin-1-yl)-quinazoline;
4-(2,6-Difluoro-phenyl)-7-fluoro-2-(4-methyl-piperazin-1-yl)-quinazoline;
[4-(2,6-Difluoro-phenyl)-7-fluoro-quinazolin-2-yl]-pyrrolidin-3-yl-amine;
4-(2-Chloro-6-fluoro-phenyl)-2-(2-methyl-piperazin-1-yl)-quinazoline;
4-(2,6-Difluoro-phenyl)-2-(2-methyl-piperazin-1-yl)-quinazoline;
4-(2,3-Difluoro-phenyl)-2-(2-methyl-piperazin-1-yl)-quinazoline;
4-(2,3-Difluoro-phenyl)-2-(2-methyl-piperazin-1-yl)-quinazoline;
7-Chloro-4-phenyl-2-piperazin-1-yl-quinazoline;
4-(3-methoxy-phenyl)-2-piperazin-1-yl-quinazoline;
6-Bromo-4-phenyl-2-piperazin-1-yl-quinazoline;
6-Fluoro-4-phenyl-2-piperazin-1-yl-quinazoline;
1-[4-(2,6-Difluoro-phenyl)-7-fluoro-quinazolin-2-yl]-pyrrolidin-3-ylamine;
1-[4-(2,6-Difluoro-phenyl)-7-fluoro-quinazolin-2-yl]-pyrrolidin-3-ylamine;
1-[4-(2,6-Difluoro-phenyl)-quinazolin-2-yl]-pyrrolidin-3-ylamine;
1-[4-(2,6-Difluoro-phenyl)-quinazolin-2-yl]-pyrrolidin-3-ylamine;
7-Fluoro-4-phenyl-2-piperazin-1-yl-quinazoline;
4-(2,6-Difluoro-phenyl)-7-fluoro-2-piperazin-1-yl-quinazoline;
[4-(2,6-Difluoro-phenyl)-quinazolin-2-yl]-pyrrolidin-3-yl-amine;
[4-(2,6-Difluoro-phenyl)-7-fluoro-quinazolin-2-yl]-pyrrolidin-3-yl-amine;
[4-(2,6-Difluoro-phenyl)-quinazolin-2-yl]-pyrrolidin-3-yl-amine;
1-[4-(2,3-Difluoro-phenyl)-quinazolin-2-yl]-pyrrolidin-3-ylamine;
4-(3,4-Difluoro-phenyl)-2-(4-methyl-piperazin-1-yl)-quinazoline;
4-(2,6-Difluoro-phenyl)-7-fluoro-2-(2-methyl-piperazin-1-yl)-quinazoline;
7-Fluoro-2-(2-methyl-piperazin-1-yl)-4-phenyl-quinazoline;
7-Fluoro-2-(2-methyl-piperazin-1-yl)-4-phenyl-quinazoline;
4-(2,6-Difluoro-phenyl)-7-fluoro-2-(2-methyl-piperazin-1-yl)-quinazoline;
4-(3,4-difluoro-phenyl)-2-piperazin-1-yl-quinazoline;
1-[4-(2,3-Difluoro-phenyl)-7-fluoro-quinazolin-2-yl]-pyrrolidin-3(S)-yl-amine;
1-[4-(2,3-Difluoro-phenyl)-7-fluoro-quinazolin-2-yl]-pyrrolidin-3(R)-yl-amine;
4-(2,3-Difluoro-phenyl)-7-fluoro-2-piperazin-1-yl-quinazoline;
4-(3,4-Difluoro-phenyl)-7-fluoro-2-(4-methyl-piperazin-1-yl)-quinazoline;
4-(3-chloro-phenyl)-2-piperazin-1-yl-quinazoline;
4-(3,4-Difluoro-phenyl)-2-(4-methyl-piperazin-1-yl)-quinazoline;
4-(3,4-dichloro-phenyl)-2-piperazin-1-yl-quinazoline;
[4-(2,3-Difluoro-phenyl)-7-fluoro-quinazolin-2-yl]-pyrrolidin-3-yl-amine;
7-Fluoro-4-(4-fluoro-2-methyl-phenyl)-2-piperazin-1-yl-quinazoline;
7-Chloro-4-(4-fluoro-2-methyl-phenyl)-2-piperazin-1-yl-quinazoline;
4-(3,4-Difluoro-phenyl)-7-fluoro-2-piperazin-1-yl-quinazoline;
4-(2,4-Dichloro-phenyl)-7-fluoro-2-piperazin-1-yl-quinazoline;
4-(2,3-Difluoro-phenyl)-6-fluoro-2-piperazin-1-yl-quinazoline;
4-(2,4-Difluoro-phenyl)-6-fluoro-2-piperazin-1-yl-quinazoline;
4-(2,3-Difluoro-phenyl)-6,7-difluoro-2-piperazin-1-yl-quinazoline;
4-(2,3-Difluoro-phenyl)-7-fluoro-2-(2-methyl-piperazin-1-yl)-quinazoline;
4-(2,5-dichloro-phenyl)-2-piperazin-1-yl-quinazoline;
4-(3,5-difluoro-phenyl)-2-piperazin-1-yl-quinazoline;
4-(2,6-Difluoro-phenyl)-2-(2-methyl-piperazin-1-yl)-quinazoline;
6-Chloro-4-phenyl-2-piperazin-1-yl-quinazoline;
4-(2-Fluoro-phenyl)-6-chloro-2-piperazin-1-yl-quinazoline;
4-(2,3-Difluoro-phenyl)-7-fluoro-2-(2-methyl-piperazin-1-yl)-quinazoline;
4-(2,6-Difluoro-phenyl)-6-chloro-2-piperazin-1-yl-quinazoline;
N1-[4-(2,4-Difluoro-phenyl)-quinazolin-2-yl]-ethane-1,2-diamine;
8-Fluoro-4-phenyl-2-piperazin-1-yl-quinazoline;
4-(2-Chloro-4-fluoro-phenyl)-7-fluoro-2-piperazin-1-yl-quinazoline;
7-Fluoro-2-piperazin-1-yl-4-thiazol-2-yl-quinazoline;
4-(2,-Methoxy-phenyl)-7-fluoro-2-piperazin-1-yl-quinazoline;
7-Fluoro-4-(5-fluoro-2-methyl-phenyl)-2-piperazin-1-yl-quinazoline;
4-(2,4-Difluoro-phenyl)-7-fluoro-2-(2-methyl-piperazin-1-yl)-quinazoline;
4-(2,4-Difluoro-phenyl)-7-fluoro-2-(2-methyl-piperazin-1-yl)-quinazoline;
4-(2,4-Difluoro-phenyl)-7-fluoro-2-piperazin-1-yl-quinazoline;
Azetidin-3-yl-[4-(2,4-difluoro-phenyl)-7-fluoro-quinazolin-2-yl]-amine;
4-(2,4-Difluoro-phenyl)-7-fluoro-2-(hexahydro-pyrrolo[3,4-b]pyrrol-1-yl)-quinazoline;
[4-(2,4-Difluoro-phenyl)-7-fluoro-quinazolin-2-yl]-pyrrolidin-3-yl-amine;
[4-(2,4-Difluoro-phenyl)-7-fluoro-quinazolin-2-yl]-pyrrolidin-3-yl-amine;
5-Methyl-4-phenyl-2-piperazin-1-yl-quinazoline;
4-(2,6-Difluoro-phenyl)-6,7-difluoro-2-piperazin-1-yl-quinazoline;
4-(2,4-Difluoro-phenyl)-6,7-difluoro-2-piperazin-1-yl-quinazoline;
[4-(2,6-Difluoro-phenyl)-quinazolin-2-yl]-piperidin-4-yl-amine;
{1-[4-(2,6-Difluoro-phenyl)-quinazolin-2-yl]-pyrrolidin-3-yl}-methyl-amine;
{1-[4-(2,4-Difluoro-phenyl)-quinazolin-2-yl]-pyrrolidin-3-yl}-methyl-amine;
{1-[4-(2,6-Difluoro-phenyl)-quinazolin-2-yl]-piperidin-4-yl}-methyl-amine;
N1-[4-(2,4-Difluoro-phenyl)-quinazolin-2-yl]-propane-1,3-diamine;
{1-[4-(2,4-Difluoro-phenyl)-quinazolin-2-yl]-piperidin-4-yl}-methyl-amine;
[4-(2,4-Difluoro-phenyl)-quinazolin-2-yl]-piperidin-4-yl-amine;
[4-(2,4-Difluoro-phenyl)-quinazolin-2-yl]-pyrrolidin-3-ylmethyl-amine;
[4-(2,3-Difluoro-phenyl)-quinazolin-2-yl]-piperidin-4-yl-amine;
{1-[4-(2,3-Difluoro-phenyl)-quinazolin-2-yl]-pyrrolidin-3-yl}-methyl-amine;
{1-[4-(2,3-Difluoro-phenyl)-quinazolin-2-yl]-piperidin-4-yl}-methyl-amine;
N1-[4-(2,3-Difluoro-phenyl)-quinazolin-2-yl]-propane-1,3-diamine;
7-Fluoro-2-piperazin-1-yl-4-(2-trifluoromethyl-phenyl)-quinazoline;
2-(2,4-Difluoro-phenyl)-4-piperazin-1-yl-quinazoline;
4-(2,6-Difluoro-phenyl)-7-fluoro-2-piperidin-4-yl-quinazoline;
7,8-Difluoro-4-phenyl-2-piperazin-1-yl-quinazoline;
4-(2,6-Difluoro-phenyl)-2-(2-ethyl-piperazin-1-yl)-quinazoline;
4-(2,4-Difluoro-phenyl)-2-(2-ethyl-piperazin-1-yl)-quinazoline;
4-(2,3-Difluoro-phenyl)-2-(2-isopropyl-piperazin-1-yl)-quinazoline; and
4-(2,4-Difluoro-phenyl)-2-(2-isopropyl-piperazin-1-yl)-quinazoline.

Preferred compounds of the invention also include the following compounds and their solvates and hydrates:
4-Isopropyl-2-piperazin-1-yl-quinazoline hydrochloride;
2-[1,4]Diazepan-1-yl-4-phenyl-quinazoline hydrochloride;
2-[1,4]Diazepan-1-yl-4-isopropyl-quinazoline hydrochloride;
2-(2,5-Dimethyl-piperazin-1-yl)-4-phenyl-quinazoline hydrochloride;
2-(2,5-Diaza-bicyclo[2.2.1]hept-2-yl)-4-phenyl-quinazoline hydrochloride;
2-[1-(4-Phenyl-quinazolin-2-yl)-piperidin-3-yl]-ethylamine hydrochloride;
1-(4-Phenyl-quinazolin-2-yl)-piperidin-4-ylamine hydrochloride;
N1-(4-Phenyl-quinazolin-2-yl)-ethane-1,2-diamine hydrochloride;
1-(4-Phenyl-quinazolin-2-yl)-pyrrolidin-3-ylamine hydrochloride;
2-(2-Methyl-piperazin-1-yl)-4-phenyl-quinazoline hydrochloride;
1-(4-Phenyl-quinazolin-2-yl)-pyrrolidin-3-yl amine hydrochloride;
3-(4-Phenyl-quinazolin-2-yl)-aminopyrrolidine hydrochloride;
4-(2-Fluoro-phenyl)-2-piperazin-1-yl-quinazoline hydrochloride;
2-[1,4]Diazepan-1-yl-4-(2-fluoro-phenyl)-quinazoline hydrochloride;
4-(2-Chloro-phenyl)-2-piperazin-1-yl-quinazoline hydrochloride;
4-(2-Methoxy-phenyl)-2-piperazin-1-yl-quinazoline hydrochloride;
4-(2-Methyl-phenyl-2-piperazin-1-yl)quinazoline hydrochloride;
2-(4-Methyl-piperazin-1-yl)-4-thiophen-2-yl-quinazoline oxalate;
4-Benzyl-2-piperazin-1-yl-quinazoline hydrochloride;
4-(2,6-Difluoro-phenyl)-2-piperazin-4-yl-quinazoline hydrochloride;
(R)-(−)-2-(2-Methyl-piperazin-1-yl)-4-phenyl-quinazoline hydrochloride;
2-(3,9-Diaza-bicyclo[3.3.1]non-3-yl)-4-phenyl-quinazoline hydrochloride;
2-(3,9-Diaza-bicyclo[3.3.1]non-3-yl)-4-(2-fluoro-phenyl)-quinazoline hydrochloride;
(S)-(+)-1-[4-(2-Fluoro-phenyl)-quinazolin-2-yl]-pyrrolidin-3-ylamine hydrochloride;
(S)-(+)-{1-[4-(2-Fluoro-phenyl)-quinazolin-2-yl]-pyrrolidin-3-yl }-methyl-amine hydrochloride;
4-(2-Chloro-6-fluoro-phenyl)-2-piperazin-1-yl-quinazoline hydrochloride;
4-(2,3-Difluoro-phenyl)-2-piperazin-1-yl-quinazoline hydrochloride;
4-(2,4-Difluoro-phenyl)-2-piperazin-1-yl-quinazoline hydrochloride;
4-(2-Fluoro-phenyl)-2-(hexahydro-pyrrolo[3,4-c]pyrrol-2-yl)-quinazoline hydrochloride;
(S)-(+)-1-[4-(2-Fluoro-phenyl)-quinazolin-2-yl]-piperidin-3-ylamine hydrochloride;
4-(2-Fluoro-phenyl)-2-(piperidin-4-yl)-quinazoline hydrochloride;
4-Phenyl-2-piperidin-4-yl-quinazoline hydrochloride;
4-(2-Chloro-phenyl)-2-piperidin-4-yl-quinazoline hydrochloride;
4-(2-Methoxy-phenyl)-2-piperidin-4-yl-quinazoline hydrochloride;
4-(2-Methyl-phenyl)-2-piperidin-4-yl-quinazoline hydrochloride;
4-Phenyl-2-piperidin-3-yl-quinazoline hydrochloride;
4-(4-Phenyl-quinazolin-2-yl)piperidine-4-carboxylic acid methyl ester hydrochloride;
4-[4-(2-Fluoro-phenyl)-quinazolin-2-yl]-piperidine-4-carboxylic acid methyl ester hydrochloride; and
3-(4-Phenyl-quinazolin-2-yl)-piperidine-3-carboxylic acid methyl ester hydrochloride; and
2-Piperazin-1-yl-4-s-tolyl-quinazoline hydrochloride.

The present invention also provides a method of treating attention deficit hyperactivity disorder (ADHD) comprising administering to a mammal in need of such treatment a therapeutically effective amount of a compound having the formula 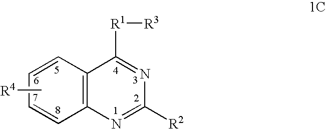
or a pharmaceutically acceptable salt, solvate or hydrate thereof, wherein
R¹ is (C₁-C₆)alkyl, (C₃-C₈)cycloalkyl, (C₁-C₆)alkoxy, aryl, amino, halogen, hydroxy, or a saturated, unsaturated, or aromatic five to seven membered monocyclic heterocyclic ring containing from one to three heteroatoms independently selected from oxygen, nitrogen, and sulfur;
R² is (C₁-C₆)alkyl, (C₃-C₈)cycloalkyl, or a saturated, unsaturated, or aromatic five to seven membered monocyclic heterocyclic ring or a six to ten membered bicyclic ring containing from one to three heteroatoms independently selected from oxygen, nitrogen, and sulfur, any of which may be unsubstituted or substituted with one or more of the following substituents: (C₁-C₆)alkyl, amino, (C₁-C₆)alkylamino, or a heterocyclic group;
R³ is independently selected from one or more of hydrogen, (C₁-C₆)alkyl, (C₃-C₈)cycloalkyl, (C₁-C₆)alkyl, amino, (C₁-C₆)alkylamino, or a heterocyclic group; and
R⁴ is a hydrogen, halogen, —NO₂, (C₁-C₆)alkyl, (C₁-C₆) alkoxy, or a heterocyclic group.

Certain compounds of formula 1C are disclosed in U.S. Pat. Nos. 4,499,092 and 4,540,696, both of which are incorporated herein by reference.

Another aspect of this invention relates to compounds and their pharmaceutically acceptable salts, solvates, and hydrates of formula 1D 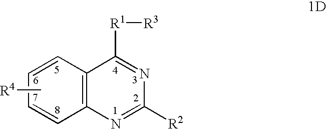
wherein
R¹ is (C₁-C₆)alkyl, (C₃-C₈)cycloalkyl, (C₁-C₆)alkoxy; aryl, amino, halogen, hydroxy, or a saturated, unsaturated, or aromatic five to seven membered monocyclic heterocyclic ring containing from one to three heteroatoms independently selected from oxygen, nitrogen, and sulfur;
R² is piperidinyl unsubstituted or substituted with one or more of the following substituents: (C₁-C₆)alkyl, amino, (C₁-C₆)alkylamino, a heterocyclic group or a carboxylic acid or ester thereof;
R³ is independently selected from one or more of hydrogen, (C₁-C₆)alkyl, (C₃-C₈)cycloalkyl, (C₁-C₆)alkoxy, aryl, amino, halogen, or hydroxy groups; and
R⁴ is a hydrogen, halogen, —NO₂, (C₁-C₆)alkyl, (C₁-C₆) alkoxy, or a heterocyclic group, with the proviso that when R¹ is phenyl and R² is 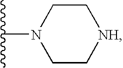
   then R⁴ cannot be halogen, methyl, or NO₂ at the 6-position.

Preferred compounds of this invention are compounds of formula 1D including:
4-(4-Phenyl-quinazolin-2-yl)piperidine-4-carboxylic acid methyl ester hydrochloride;
4-Phenyl-2-piperidin-4-yl-quinazoline hydrochloride;
4-(2-Fluoro-phenyl)-2-(1-methyl-piperidin-4-yl)-quinazoline;
4-(2-Chloro-phenyl)-2-(1-methyl-piperidin-4-yl)-quinazoline;
4-[4-(2-Fluoro-phenyl)-quinazolin-2-yl]-piperidine-4-carboxylic acid methyl ester;
4-Phenyl-2-piperidin-3-yl-quinazoline;
4-(2-Chloro-phenyl)-2-piperidin-4-yl-quinazoline;
4-(2-Methoxy-phenyl)-2-piperidin-4-yl-quinazoline; and
3-(4-Phenyl-quinazolin-2-yl)-piperidine-3-carboxylic acid methyl ester;

In some of the following definitions, a dash (“-”) may be use used indicate a bond between atoms or point of attachment.

The term “alkyl”, as used herein, unless otherwise indicated, includes saturated monovalent hydrocarbon radicals having straight, branched or cyclic moieties or combinations thereof, which may be unsubstituted or substituted with one or more of the substituents listed below for aryl. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “aryl” means an aromatic carbocyclic group having a single ring (e.g., phenyl), multiple rings (e.g., biphenyl), or multiple condensed rings in which at least one is aromatic (e.g., 1,2,3,4-tetrahydronaphthyl, naphthyl, anthryl, or phenanthryl), unsubstituted or substituted by 1 to 3 substituents selected from alkyl, O-alkyl and S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —CH₂)ₘPO₃H₂, —(CH₂)ₘPO3(alkyl)₂, —(CH₂)ₘSO₂NH₂, and —(CH₂)ₘSO₂NH-alkyl wherein alkyl is defined as above and m is 0, 1, 2, or 3. A preferable aryl group of the present invention is phenyl. Typical substituted phenyl groups include 2-chlorophenyl, 3-methoxyphenyl, 4-aminophenyl, 3,5-dinitrophenyl, 2,6-dibromo-4-ethoxyphenyl, and 2-hydroxy-3-cyano-5-trifluoromethylphenyl.

The term “alkoxy”, as used herein, unless otherwise indicated, means “alkyl-O—”, wherein “alkyl” is as defined above. Examples of “alkoxy” groups include, but are not limited to, methoxy, ethoxy, propoxy, butoxy and pentoxy.

The term “heteroaryl” as used herein, unless otherwise indicated, includes monocyclic aromatic heterocycles containing five to seven ring members, of which from 1 to 4 can be heteroatoms selected, independently, from N, S and O, and bicyclic aromatic heterocycles containing from six to 10 ring members, of which from 1 to 4 can be heteroatoms selected, independently, from N, S and O.

The term “heterocycle”, as used herein, unless otherwise indicated, means a 5- to 10-membered mono- or bicyclic ring structure which may contain one or more heteroatoms such as N or O; examples of heterocycles are pyridine, pyrimidine, pyridazine, pyrazole, oxazole, indole, N-alkylindole, quinoline, quinazoline, and the like.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above.

“Pharmaceutically acceptable salts” refers to acid or base addition salts of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

The compounds of the present invention also include prodrugs thereof. “Prodrugs” refer to compounds having little or no pharmacological activity that can, when metabolized in vivo, undergo conversion to claimed or disclosed compounds having desired activity. For a discussion of prodrugs, see T. Higuchi and V. Stella, “Pro-drugs as Novel Delivery Systems,” ACS Symposium Series 14 (1975), E. B. Roche (ed.), Bioreversible Carriers in Drug Design (1987), and H. Bundgaar, Design of Prodrugs (1985).

The compounds of formulae 1, 1B, 1C, or 1D and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formulae 1, 1B, 1C, or 1D or a pharmaceutically acceptable salt thereof, and a pharmaceutically acceptable carrier.

Compounds of formulae 1, 1B, 1C, or 1D may contain chiral centers and therefore may exist in different enantiomeric and diastereomeric forms. This invention relates to all optical isomers and all stereoisomers of compounds of the formulae 1, 1B, 1C, or 1D, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, optically selective reaction, or chromatographic separation in the preparation of the final product or its intermediate. Individual enantiomers of the compounds of formulae 1, 1B, 1C, or 1D may have advantages, as compared with the racemic mixtures of these compounds, in the treatment of various disorders or conditions.

In so far as the compounds of formulae 1, 1B, 1C, or 1D of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, oxalate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (ie., 1,1′-methylene-bis-(2-hydroxy-3-naphthoate)) salts.

The present invention also includes isotopically labeled compounds, which are identical to those recited in formulae 1, 1B, 1C, or 1D, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formulae 1, 1B, 1C, or 1D of this invention and prodrugs thereof can generally be prepared by carrying out the procedures disclosed in the Schemes and/or in the Examples below, by substituting a readily available isotopically labeled reagent for a non-isotopically labeled reagent.

The compounds of formulae 1, 1B, 1C, or 1D of this invention have useful pharmaceutical and medicinal properties.

Compounds of formulae 1, 1B, 1C, or 1D of this invention in addition to the inhibition of norepinephrine reuptake also possess activity as inhibitors or antagonists of 5-hydroxytryptamine-3-receptor (5-HT3).

This invention also relates to a method of treating a disorder or condition selected from the group consisting of norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression or reactive depression including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias including specific animal phobias, social anxiety, social phobia including social anxiety disorder, obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders including schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions including dependencies on, or addictions to, alcohol, heroin, cocaine, benzodiazepines, psychoactive substances, nicotine, or phenobarbitol and behavioral addictions including addiction to gambling; ocular disorders such as glaucoma and ischemic retinopathy addictive disorders including those due to alcohol, nicotine, and other psychoactive substances and withdrawal syndrome, adjustment disorders and disruptive behavior disorder including depressed mood, anxiety, mixed anxiety and depressed mood, disturbance of conduct, and mixed disturbance of conduct and mood; age-associated learning and mental disorders including Alzheimer's disease; anorexia nervosa; apathy; attention-deficit or other cognitive disorders due to general medical conditions including attention-deficit disorder (ADD) and attention-deficit hyperactivity disorder (ADHD) and it's recognized sub-types; reading disorders; bulimia nervosa; chronic fatigue syndrome; pain; chronic pain; cyclothymic disorder; depression including adolescent depression and minor depression; fibromyalgia and other somatoform disorders including somatization disorder; conversion disorder; pain disorder; hypochondriasis; body dysmorphic disorder; undifferentiated somatoform disorder; and somatoform NOS; incontinence including stress incontinence; genuine stress incontinence; and mixed incontinence; urinary disorders; premature ejaculation; inhalation disorders; intoxication disorders including alcohol addiction; mania; migraine headaches; obesity including reducing the weight of obese or overweight patients; restless legs syndrome; oppositional defiant disorder; peripheral neuropathy; diabetic neuropathy; post-herpetic neuralgic; premenstrual dysphoric disorder including premenstrual syndrome and late luteal phase dysphoric disorder; hot flashes; sleep disorders including narcolepsy, insomnia, enuresis, sleep walking disorder and breathing related disorder; specific developmental disorders; selective serotonin reuptake inhibition (SSRI) “poop out” syndrome; and TIC disorders including Tourette's Disease in a mammal, including a human, comprising administering to a mammal in need of such treatment an amount of a compound of the formulae 1, 1B, 1C, or 1D, or a pharmaceutically acceptable salt thereof, that is effective in treating such disorder or condition.

The compounds of formulae 1, 1B, 1C, or 1D and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formulae 1, 1B, 1C, or 1D, or a pharmaceutically acceptable salt thereof, and a pharmaceutically acceptable carrier.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; pain; stress induced urinary incontinence; premature ejaculation; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising an amount of a compound of the formulae 1, 1B, 1C, or 1D or a pharmaceutically acceptable salt thereof, that is effective in treating such disorder or condition, and a pharmaceutically acceptable carrier.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is pain. Pain refers to acute as well as chronic pain. Acute pain is usually short-lived and is associated with hyperactivity of the sympathetic nervous system. Examples are postoperative pain and allodynia. Chronic pain is usually defined as pain persisting from 3 to 6 months and includes somatogenic pain and psychogenic pain. Other pain is nociceptive.

Examples of the types of pain that can be treated with the compounds of formulas 1, 1B, 1C, or 1D of the present invention and their pharmaceutically acceptable salts include pain resulting from soft tissue and peripheral damage, such as acute trauma, pain associated with osteoarthritis and rheumatoid arthritis, musculo-skeletal pain, such as pain experienced after trauma; spinal pain, dental pain, myofascial pain syndromes, episiotomy pain, and pain resulting from burns; deep and visceral pain, such as heart pain, muscle pain, eye pain, orofacial pain, for example, odontalgia, abdominal pain, gynaecological pain, for example, dysmenorrhoea, labour pain and pain associated with endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, trigeminal neuralgia, neuropathic lower back pain, HIV related neuropathic pain, cancer related neuropathic pain, diabetic neuropathic pain, and arachnoiditis; neuropathic and non-neuropathic pain associated with carcinoma, often referred to as cancer pain; central nervous system pain, such as pain due to spinal cord or brain stem damage; lower back pain; sciatica; phantom limb pain, headache, including migraine and other vascular headaches, acute or chronic tension headache, cluster headache, temperomandibular pain and maxillary sinus pain; pain resulting from ankylosing spondylitis and gout; pain caused by increased bladder contractions; post operative pain; scar pain; and chronic non-neuropathic pain such as pain associated with fibromyalgia, HIV, rheumatoid and osteoarthritis, arthralgia and myalgia, sprains, strains and trauma such as broken bones; and post surgical pain.

Still other pain is caused by injury or infection of peripheral sensory nerves. It includes, but is not limited to pain from peripheral nerve trauma, herpes virus infection, diabetes mellitus, fibromyalgia, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Neuropathic pain includes, but is not limited to pain caused by nerve injury such as, for example, the pain diabetics suffer from.

Psychogenic pain is that which occurs without an organic origin such as low back pain, atypical facial pain, and chronic headache.

Other types of pain are: inflammatory pain, osteoarthritic pain, trigeminal neuralgia, cancer pain, diabetic neuropathy, restless leg syndrome, acute herpetic and postherpetic neuralgia, causalgia, brachial plexus avulsion, occipital neuralgia, gout, phantom limb, burn, and other forms of neuralgia, neuropathic and idiopathic pain syndrome.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compound of formulae 1, 1B, 1C, or 1D is administered to a human for the treatment of any two or more comorbid disorders or conditions selected from those disorders and conditions referred to in any of the above methods.

For the treatment of depression, anxiety, schizophrenia or any of the other disorders and conditions referred to above in the descriptions of the methods and pharmaceutical compositions of this invention, the novel compounds of this invention can be used in conjunction with one or more other antidepressants or anti-anxiety agents. Examples of classes of antidepressants that can be used in combination with the active compounds of this invention include norepinephrine reuptake inhibitors, selective serotonin reuptake inhibitors (SRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, alpha-2-delta ligands (A2D), and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine, citalopram, and sertraline. Examples of monoamine oxidase inhibitors include isocarboxazid, phenelzine, and tranylcyclopramine. Suitable reversible inhibitors of monoamine oxidase include moclobemide. Suitable serotonin and noradrenaline reuptake inhibitors of use in the present invention include venlafaxine and duloxetine. Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in World Patent Publication WO 01/77100. Suitable A2D ligands include those referred to in World Patent Publications WO 99/21824, WO 01/90052, WO 01/28978, WO 98/17627, WO 00/76958, and WO 03/082807, and specifically gabapentin and pregabalin.

Suitable classes of anti-anxiety agents that can be used in combination with the active compounds of this invention include benzodiazepines and serotonin IA (5-HT_{1A}) agonists or antagonists, especially 5-HT_{1A} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

Suitable antipsychotic agents include both conventional and atypical antipsychotics.

Conventional antipsychotics are antagonists of dopamine (D₂) receptors. The atypical antipsychotics also have D₂ antagonistic properties but possess different binding kinetics to these receptors and activity at other receptors, particularly 5-HT_{2A}, 5-HT_{2C} and 5-HT_{2D} (Schmidt B et al, Soc. Neurosci. Abstr. 24:2177, 1998).

The class of atypical antipsychotics includes clozapine (Clozaril®), 8-chloro-11-(4-methyl-1-piperazinyl)-5H-dibenzo[b,e][1,4]diazepine (U.S. Pat. No. 3,539,573); risperidone (Risperdal®), 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (U.S. Pat. No. 4,804,663); olanzapine (Zyprexa®), 2-methyl-4-(4-methyl-1-piperazinyl)-10H-thieno[2,3-b][1,5]benzodiazepine (U.S. Pat. No. 5,229,382); quetiapine (Seroquel®), 5-[2-(4-dibenzo[b,f][1,4]thiazepin-11-yl-1-piperazinyl)ethoxy]ethanol (U.S. Pat. No. 4,879,288); aripiprazole (Abilify®), 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro-2(1H)-quinolinone (U.S. Pat. Nos. 4,734,416 and 5,006,528); sertindole, 1-[2-[4-[5-chloro-1-(4-fluorophenyl)-1H-indol-3-yl]-1-piperidinyl]ethyl]imidazolidin-2-one (U.S. Pat. No. 4,710,500); amisulpride (U.S. Pat. No. 4,410,822); asenapine (U.S. Pat. No. 4,415,434); and ziprasidone (Geodon®), 5-[2-[4-(1,2-benzisothiazol-3-yl)piperazin-3-yl]ethyl]-6-chloroindolin-2-one hydrochloride hydrate (U.S. Pat. No. 4,831,031).

Compounds of the present invention may also be administered with one or more compounds such as NEURONTIN®, LYRICA®, a tricyclic antidepressant, Amitryptyline, Fluoxetine (PROZAC®), Ibuprofen, an opioid, morphine, Fentanyl, Paroxetine, Diazepam, Femoxetine, Diazepam, Carbamazepine, Milnacipran, Venlafaxine, Duloxetine, Topisetron, Interferon alpha, Cyclobenzaprine, CELEXA™, ZOLOFT® (sertraline HCl), a muscle relaxant, or a COX-2 inhibitor, such as CELEBREX® (celecoxib), VIOXX® (rofecoxib), BEXTRA® (valdecoxib) and etoricoxib.

This invention also relates to a method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; pain; stress induced urinary incontinence; premature ejaculation; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising administering to said mammal:
(a) a compound of the formulae 1, 1B, 1C, or 1D or a pharmaceutically acceptable salt thereof; and
(b) another pharmaceutically active compound that is an antidepressant or anti-anxiety agent, or a pharmaceutically acceptable salt thereof;
wherein the active compounds “a” and “b” are present in amounts that render the combination effective in treating such disorder or condition.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compounds of formulae 1, 1B, 1C, or 1D and the additional antidepressant or anti-anxiety agent are administered to a human for the treatment of any two or more comorbid disorders or conditions selected from those disorders and conditions referred to in any of the above methods.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising:
(a) a compound of the formulae 1, 1B, 1C, or 1D or a pharmaceutically acceptable salt thereof;
(b) another pharmaceutically active compound that is an antidepressant or anti-anxiety agent, or a pharmaceutically acceptable salt thereof; and
(c) a pharmaceutically acceptable carrier;

wherein the active compounds “a” and “b” are present in amounts that render the composition effective in treating such disorder or condition.

### DETAILED DESCRIPTION OF THE INVENTION

The compounds of formulae 1, 1B, 1C, or 1D of the present invention may be prepared as described in the following reaction schemes. Unless otherwise indicated, R¹-R⁴ in the reaction schemes and discussion that follow, are as defined above.

### General Synthesis

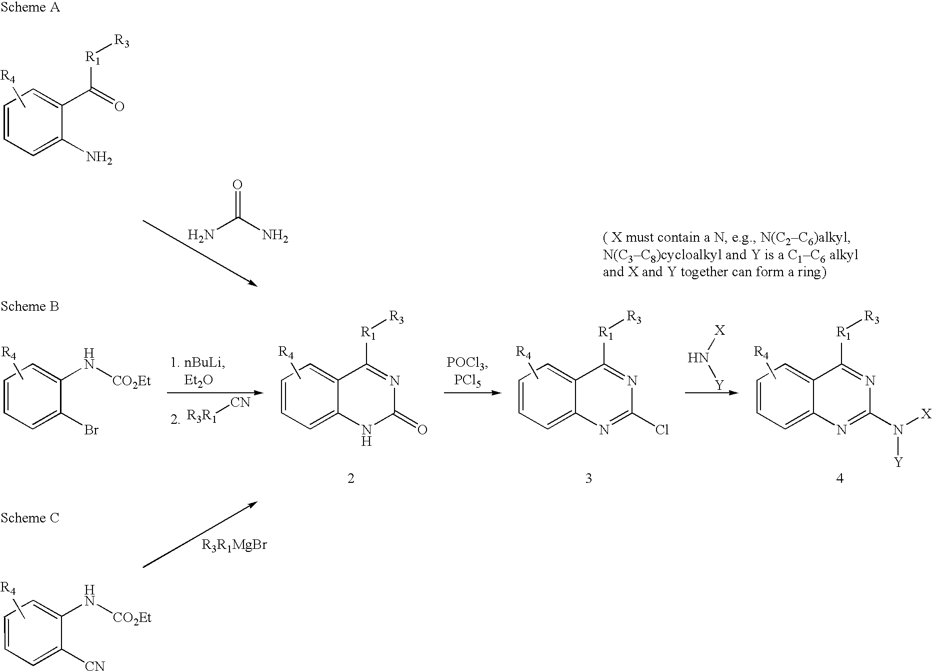

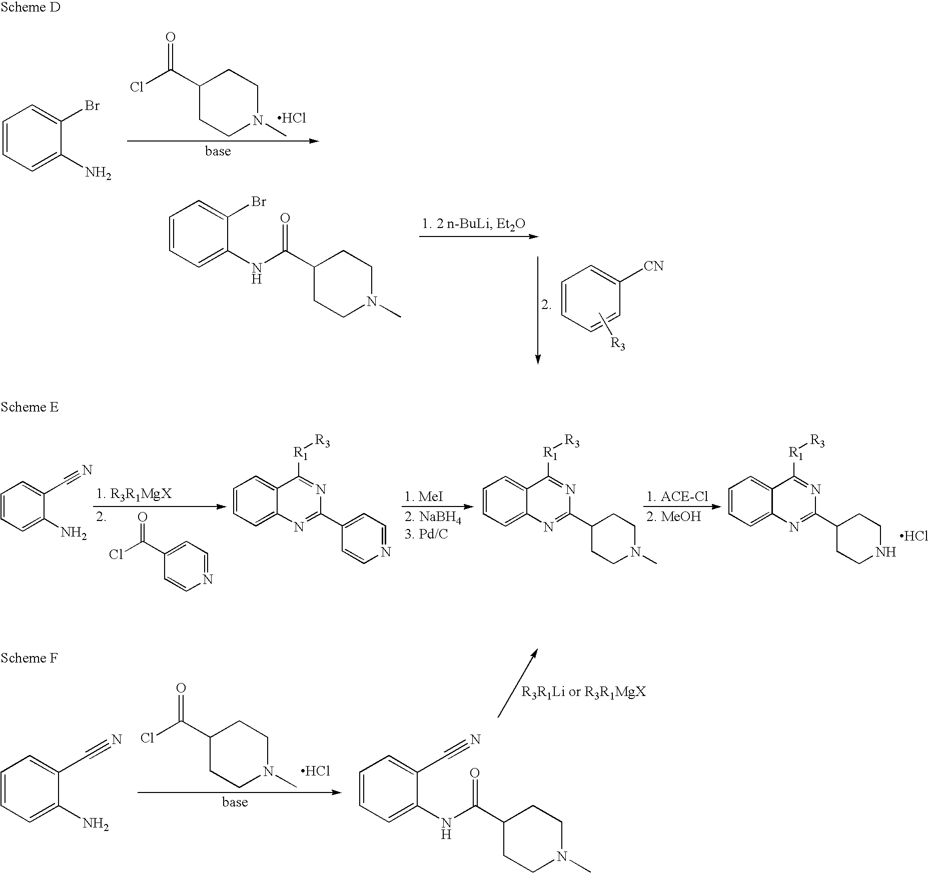

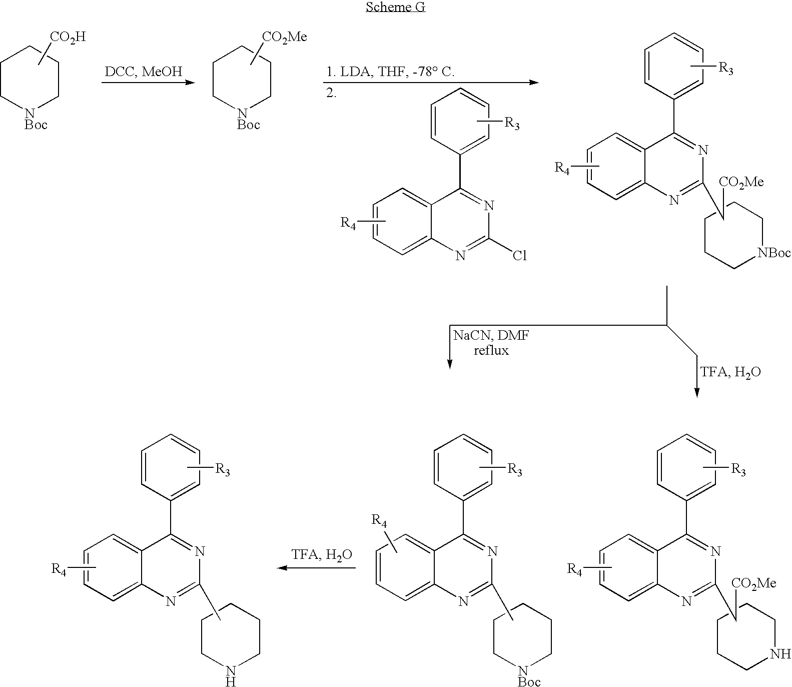

Compounds of the present invention 4 can be prepared by reaction of an amine with an appropriately 4-substituted 2-chloro-quinazoline 3, which is obtained by chlorination of a 4-substituted quinazolin-2-one 2 with phosphorous oxychloride and, in some cases together with phosphorus pentachloride. This quinazolin-2-one can be prepared from 2-aminobenzophenone and urea (Scheme A), an appropriately substituted benzonitrile with the lithiated ethyl carbamate of 2-bromoaniline (Scheme B) or from (2-cyano-phenyl)-carbamic acid ethyl ester and a Grignard reagent (Scheme C). Alternatively, one can lithiate an amide and add to a nitrile to form the quinazoline (Method D) or add a lithiate or Grignard to an amido nitrile (Schemes E and F). Finally, one can add a lithiate to an appropriately 4-substituted 2-chloro-quinazoline 3 to form the desired 2-substituted quinazoline (Scheme G).

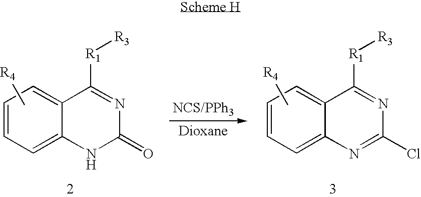

Scheme H depicts an alternative reaction to the conversion of 2 to 3. Triphenyl phosphine (PPh₃) is added to N-chlorosuccinimide (NCS) in dioxane. The reaction mixture is stirred and 2 (e.g., 4-(2,4-difluoro-phenyl)-7-fluoro-1H-quinazolin-2-one) is added to provide 3 (e.g., 2-Chloro-4-(2,4-difluoro-phenyl)-7-fluoro-quinazoline).

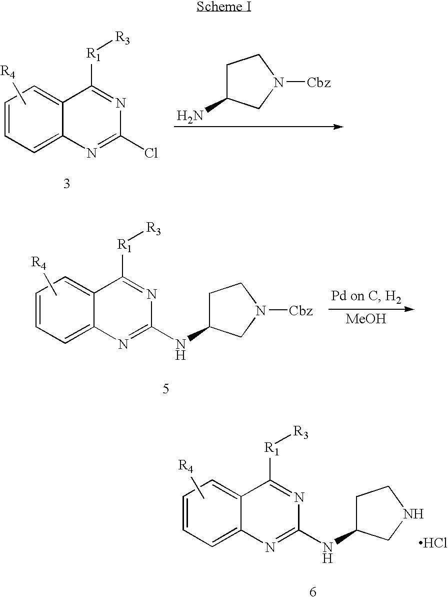

In Scheme I, a benzyloxycarbonyl (Cbz) protected amino pyrrolidine (e.g., (S)-1-Cbz-3-aminopyrrolidine) or benzyl protected piperazine (e.g., 1-benzyl-3-isopropylpiperazine or 1-benzyl-3-ethylpiperazine) is reacted with 3 (e.g., 2-chloro-4-(2,6-difluoro-phenyl)-quinazoline) in toluene to provide 5 (e.g., 3-[4-(2,6-Difluoro-phenyl)-quinazolin-2-ylamino]-pyrrolidine-1-carboxylic acid benzyl ester). The Cbz group or benzyl group of 5 is then removed by reaction with hydrogen gas over palladium on carbon in methanol.

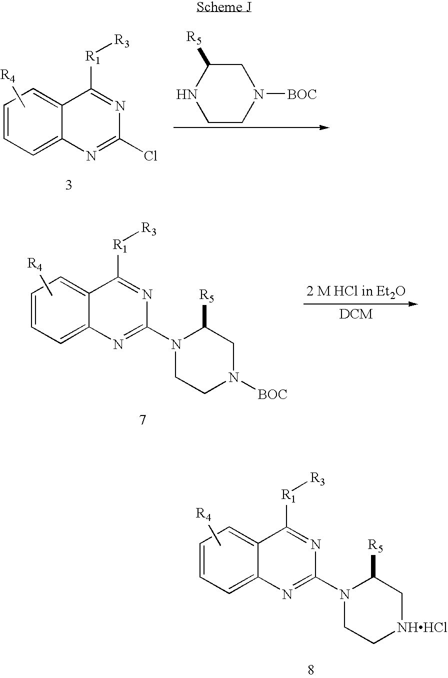

In Scheme J, 3 (e.g., 2-Chloro-4-(2,4-difluoro-phenyl)-7-fluoro-quinazoline) can be reacted with a t-butoxycarbonyl (BOC) protected piperazine (e.g., (S)-4-N-BOC-2-methylpiperazine) to afford 7 (e.g., 4-[4-(2,4-Difluoro-phenyl)-7-fluoro-quinazolin-2-yl]-3-methyl-piperazine-1-carboxylic acid tert-butyl ester). The BOC group of 7 can then be removed in a solvent such as dichloromethane (DCM) by treatment with an acid such as HCl to provide 8.

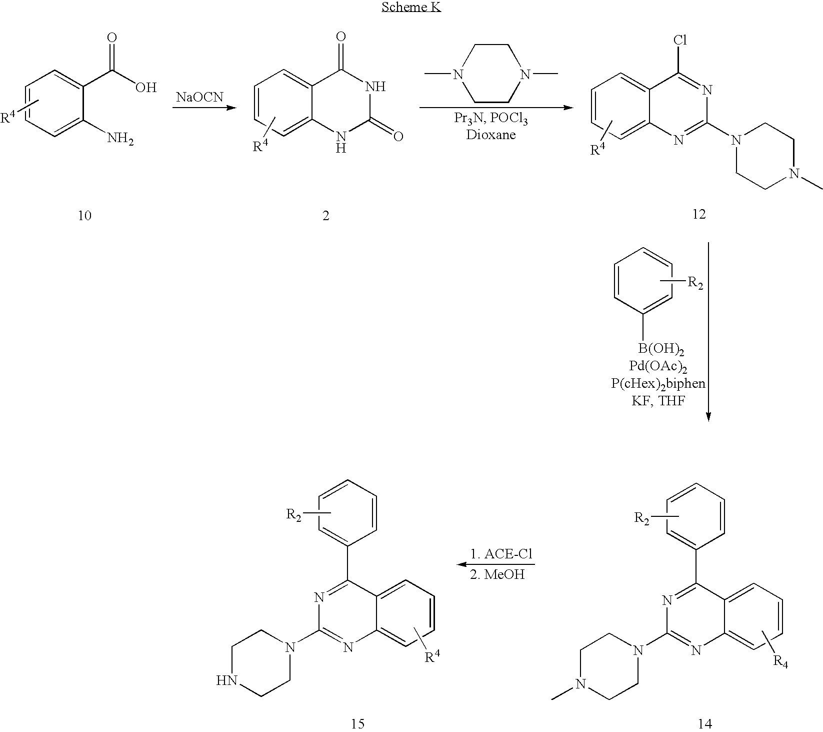

In Scheme K, a 2-amino-benzoic acid 10 (e.g., 2-Amino-4-fluoro-benzoic acid) in water and glacial acetic acid is treated with a suspension of sodium cyanate in water. The reaction is treated with base (e.g., sodium hydroxide ) to provide 2 (e.g., 7-Fluoro-1H-quinazoline-2,4-dione). The quinazoline dione 2, dimethylpiperazine, and a tertiary amine (e.g., tripropylamine), in dioxane are treated with phosphorous oxychloride to afford 12 (e.g., 4-Chloro-7-fluoro-2-(4-methyl-piperazin-1-yl)-quinazoline). An aryl boronic acid (e.g., a phenyl boronic acid, 3,4-difluoroboronic acid), 12, potassium fluoride, palladium acetate (Pd(OAc)₂) and dicyclohexylphosphinobiphenyl (P(cHex)₂biphen) are dissolved in THF (tetrahydrofuran). NaOH and dichloromethane are added and the biphasic mixture was stirred to give 14 (e.g., 4-(3,4-Difluoro-phenyl)-7-fluoro-2-(4-methyl-piperazin-1-yl)-quinazoline). Then 14 in dichloroethane can be treated with proton sponge (1,8-bis(dimethylamino)naphthalene), 1-chloroethyl chloroformate (ACE-Cl) and heated to reflux in the presence of methanol to afford 15 (e.g., 4-(3,4-Difluoro-phenyl)-7-fluoro-2-piperazin-1-yl-quinazoline).

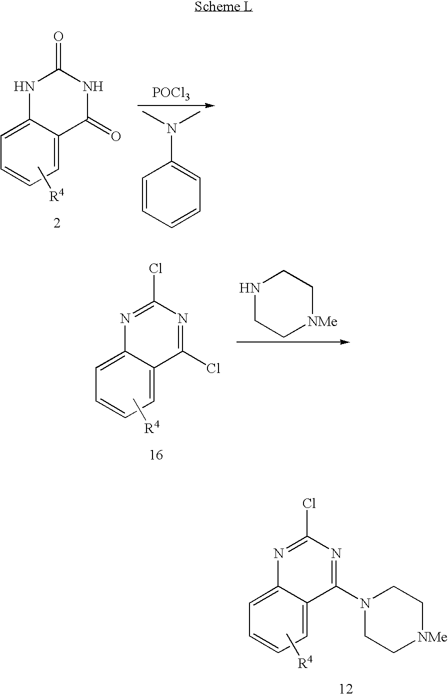

In addition to Scheme K, 12 also may be prepared as depicted in Scheme L. A quinazoline-2,4-dione 2 is dissolved in phosphorous oxychloride and treated slowly with dimethylaniline to afford the 2,4-dichloro-quinazoline 16, which is treated with methylpiperazine, followed by dichloromethane (DCM) and base (e.g., NaOH) to yield 12 (e.g., 2-Chloro4-(4-methyl-piperazin-1-yl)-quinazoline).

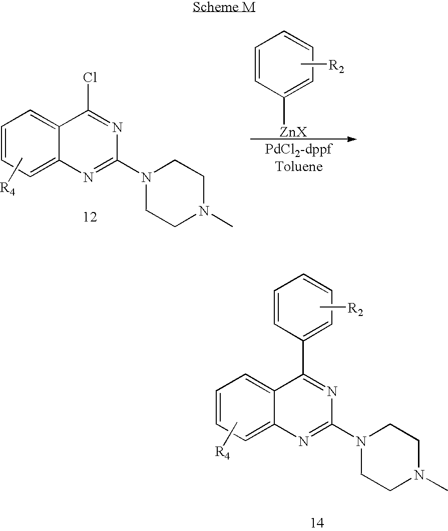

Scheme M provides for an additional manner to provide 14 from 12 using Negishi reaction conditions. A phenyl zinc halide (R²-phenyl-Zn—X, where X is a halo group) (e.g., 2-chloro4-fluorophenylzinc iodide) in tetrahydrofuran and a catalytic amount of 1,1-bis(diphenylphosphino)ferrocenepalladium(II) chloride (PdCl₂-dppf) are added to a suspension of 12 (e.g., 4-Chloro-7-fluoro-2-(4-methyl-piperazin-1-yl)-quinazoline) in toluene. The reaction is heated to reflux to afford 14 (e.g., 4-(2-Chloro4-fluoro-phenyl)-7-fluoro-2-(4-methyl-piperazin-1-yl)-quinazoline).

The preparation of other compounds of formulae 1, 1B, 1C, or 1D and intermediates used in their synthesis that are not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formulae 1, 1B, 1C, or 1D and the intermediates shown in the above reaction schemes can be isolated and purified by conventional procedures, such as recrystallization or chromatographic separation.

The compounds of the formulae 1, 1B, 1C, or 1D and their pharmaceutically acceptable salts can be administered to mammals via routes such as oral, parenteral (such as subcutaneous, intravenous, intramuscular, intrasternal and infusion techniques), rectal, buccal, and intranasal routes. In general, these compounds are most desirably administered in doses ranging from about 3 mg to about 600 mg per day, in single or divided doses (ie., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

This invention relates to methods of treating ADHD, anxiety, depression, schizophrenia and the other disorders referred to in the description of the methods of the present invention, wherein a novel compound of this invention and one or more of the other active agents referred to above (e.g., an NK1 receptor antagonist, an antipsychotic agent, tricyclic antidepressant, 5HT1B receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1B receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1B receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

### EXAMPLES

### Example 1

### 2-(4-Methyl-piperazin-1-yl)-4-phenyl-quinazoline

### Step A: 4-Phenyl-1H-quinazolin-2-one

2-Aminobenzophenone (100 g, 0.5 mole) and urea (60.8 g, 1.01 mole) are heated with efficient stirring. The mixture begins to melt at 90° C. and completely solidifies after 45 minutes at 200° C. The resulting solid is cooled, washed with hot anhydrous ethanol and cooled again before filtering to give 114 g of the title compound as a white solid; mp 247-253° C.

### Step B: 2-Chloro-4-phenyl-quinazoline

Phosphorous pentachloride (106 g, 0.51 mole) is added gradually to a suspension of 4-phenyl-1H-quinazolin-2-one (114 g, 0.51 mole) in phosphorous oxychloride (500 mL) and heated to reflux for 23 hours. Excess phosphorous oxychloride (250 mL) is distilled off and the remaining residue is poured into a mixture of concentrated ammonium hydroxide and ice (5 L) and stirred for 30 minutes. The solid is filtered, washed with water and recrystallized from 95% ethanol to give 96 g of the title compound as a pale yellow solid; mp 110-112° C.

### Step C: 2-(4-Methyl-piperazin-1-yl)-4-phenyl-quinazoline

2-Chloro4-phenyl-quinazoline (48.1 g, 0.2 mole) and N-methylpiperazine (130 mL) are heated to reflux for 2 hours. The reaction mixture is cooled, diluted with water. The solid is filtered, washed with water and recrystallized from 50% ethanol/water to give 50 g of the title compound as a yellow solid; mp 101-122° C. Elemental analysis found for C₁₉H₂₀N₄: C, 75.30; H, 6.48; N, 18.37.

Examples 2-17 were prepared in a manner similar to Example 1.

### Example 2

### 2-(4-Methyl-piperazin-1-yl)-4-p-tolyl-quinazoline

Mp 85-87° C.; Elemental analysis found for C₂₀H₂₂N₄: C, 75.17; H, 6.12.

### Example 3

### 4-Phenyl-2-piperazin-1-yl-quinazoline

Mp 127-128° C.

### Example 4

### 2-(4-Methyl-piperazin-1-yl)-4-o-tolyl-quinazoline

Mp 91-94° C.; Elemental analysis found for C₂₀H₂₂N₄: C, 75.47; H, 6.72.

### Example 5

### 2-(3-Methyl-3,9-diaza-bicyclo[3.3.1]non-9-yl)-4-phenyl-quinazoline

Mp 118-121° C.; Elemental analysis found for C₂₂H₂₄N₄: C, 76.13; H, 7.17; N, 16.02.

### Example 6

### 4-Isopropyl-2-piperazin-1-yl-quinazoline hydrochloride

Mp 253-254° C.; Elemental analysis found for C₁₅H₂₀N₄.1.15 HCl.0.15 H₂O: C, 59.83; H, 7.28; N, 15.24; Cl, 13.41.

### Example 7

### 2-[1,4]Diazepan-1-yl-4-phenyl-quinazoline hydrochloride

Mp 230-231° C.; Elemental analysis found for C₁₉H₂₀N₄.1.33 HCl: C, 64.33; H, 5.98; N, 15.64; Cl, 13.01.

### Example 8

### 2-[1,4]Diazepan-1-yl-4-isopropyl-quinazoline hydrochloride

Mp 193-194° C.; Elemental analysis found for C₁₆H₂₂N₄.HCl: C, 62.35; H, 7.51; N, 18.03; Cl, 11.68.

### Example 9

### 2-(2,5-Dimethyl-piperazin-1-yl)-4-phenyl-quinazoline hydrochloride

Mp 221-222° C.

### Example 10

### 2-(2,5-Diaza-bicyclo[2.2.1]hept-2-yl)-4-phenyl-quinazoline hydrochloride

Elemental analysis found for C₁₉H₁₈N₄.1.4 HCl.0.075 H₂O: C, 61.80; H, 5.57; N, 15.13; Cl, 13.25, H₂O, 2.88.

### Example 11

### 2-[1-(4-Phenyl-quinazolin-2-yl)-piperidin-3-yl]-ethylamine hydrochloride

Mp 86-88° C.

### Example 12

### 1-(4-Phenyl-quinazolin-2-yl)-piperidin-4-ylamine hydrochloride

Mp>300° C.; Elemental analysis found for C₁₉H₂₀N₄.1.35 HCl.0.5 H₂O: C, 62.70; H, 6.00; 0N, 15.07; Cl, 13.07.

### Example 13

### N¹-(4-Phenyl-quinazolin-2-yl)-ethane-1,2-diamine hydrochloride

Elemental analysis found for C₁₆H₁₆N₄.1.15 HCl.0.66 H₂O: C, 60.73; H, 5.49; N, 17.22; Cl, 12.61.

### Example 14

### (S)-(+)-1-(4-Phenyl-quinazolin-2-yl)-pyrrolidin-3-ylamine hydrochloride

Mp 274-275° C.; Elemental analysis found for C₁₈H₁₈N₄.HCI: C, 65.21; H, 5.78; N, 16.57; Cl, 10.83.

### Example 15

### (S)-(+)-2-(2-Methyl-piperazin-1-yl)-4-phenyl-quinazoline hydrochloride

Mp 154-157° C.; Elemental analysis found for C₁₉H₂₀N₄.HCl.0.4 H₂O: C, 65.39; H, 6.15; N, 16.00; Cl, 10.19.

### Example 16

### (R)-(−)1-(4-Phenyl-quinazolin-2-yl)-pyrrolidin-3-yl-amine hydrochloride

Mp 261-265° C.; Elemental analysis found for C₁₈H₁₈N₄.1.04 HCl.0.3 H₂O: C, 64.81; H, 5.76; N, 16.66; Cl, 11.03.

### Example 17

### (S)-(−)-3-(4-Phenyl-quinazolin-2-yl)-aminopyrrolidine hydrochloride

Mp 251-253° C.; Elemental analysis found for C₁₈H₁₈N₄.HCl.0.2 H₂O: C, 65.51; H, 5.91; N, 16.73; Cl, 10.75.

### Example 18

### 4-(2-Fluoro-phenyl)-2-(4-methyl-piperazin-1-yl)-quinazoline

### Step A: 4-(2-Fluoro-phenyl)-1H-quinazolin-2-one

Ethyl chloroformate (4.2 mL, 4.39 mmole) is added dropwise to a solution of 2-bromoaniline (6.93 g, 4.03 mmole) in anhydrous pyridine (25 mL) at 0° C. The mixture is stirred at 0° C. for 30 minutes and warmed to room temperature for 3.5 hours. The reaction is concentrated in vacuo, treated with 1N hydrochloric acid (30 mL) and extracted with ethyl acetate (2×50 mL). The combined organic layers are washed with saturated sodium chloride (1×30 mL) and dried with magnesium sulfate, filtered and concentrated in vacuo. The residue is chromatographed on silica gel with 10% ethyl acetate in hexane to give 5.92 g of (2-bromo-phenyl)-carbamic acid ethyl ester.

Butyllithium in heptane (100 mL, 0.167 mole) is added dropwise to a solution of (2-bromo-phenyl)-carbamic acid ethyl ester (20.1 g, 0.0825 mole) in anhydrous diethyl ether (150 mL) at −10° C. under nitrogen gas and stirred for 20 minutes. To this, a solution of 2-fluorobenzonitrile (9.1 g, 0.0753 mole) in anhydrous ethyl ether (50 mL) is added dropwise and warmed gradually to 10° C. over 3 hours. Saturated ammonium chloride (50 mL) and water (50 mL) are added. The mixture is stirred vigorously for 20 minutes, filtered and recrystallized from 95% ethanol to give 15.1 g of the title compound as a white solid; mp 281-285° C.

### Step B: 2-Chloro-4-(2-fluoro-phenyl)-quinazoline

Prepared in a manner similar to Example 1, Step B; mp 139-141° C.

### Step C: 4-(2-Fluoro-phenyl)-2-(4-methyl-piperazin-1-yl)-quinazoline

Prepared according to Example 1, Step C. The product was recrystallized from ethanol to give the title compound as yellow needles; mp 123-124° C. Elemental analysis found for C₁₉H₁₉N₄F: C, 70.55; H, 5.90; N, 17.29; F, 5.88.

Examples 19-21 were prepared in a manner similar to Example 18.

### Example 19

### 4-(2-Chloro-phenyl)-2-(4-methyl-piperazin-1-yl)-quinazoline

Mp 116-119° C.; Elemental analysis found for C₁₈H₁₇N₄Cl: C, 64.24; H, 5.81; N, 16.42.

### Example 20

### 4-(2-Fluoro-phenyl)-2-piperazin-1-yl-quinazoline hydrochloride

Mp 276-277° C.; Elemental analysis found for C₁₈H₁₇N₄F.HCl: C, 62.79; H, 5.28; N, 15.85; Cl, 10.33; F, 5.50.

### Example 21

### 2-[1,4]Diazepan-1-yl-4-(2-fluoro-phenyl)-quinazoline hydrochloride

Mp 190-192° C.; Elemental analysis found for C₁₉H₁₉N₄F.1.1 HCl: C, 62.62; H, 5.45; N, 15.29; Cl, 10.68; F, 5.28.

### Example 22

### 4-(2-Chloro-phenyl)-2-piperazin-1-yl-quinazoline hydrochloride

1-Chloroethylchloroformate (190 mg, 1.33 mmol) was added to a solution of 2-(1-methyl-piperidin-4-yl)-4-o-chloro-quinazoline (450 mg, 1.33 mmol) and proton sponge (1,8-bis(dimethylamino)naphthalene) (156 mg, 0.73 mmol) in 20 ml of dichloroethane. The reaction was refluxed for 3 hours. Intermediate carbamate was purified by chromatographing on silica gel using 1:4 ethylacetate/hexanes as eluent. The resulting light yellow oil was dissolved in methanol (50 mL) and warmed to 60° C. 2 hours. The solvent was removed under reduced pressure to yield an off-white solid that was triturated with ethyl acetate, collected by filtration and dried in a 50° C. vacuum oven to yield 200 mg of the title compound as a white powder. Elemental analysis found for C₁₈H₁₇ClN₄.HCl.H₂O: C, 56.66; H, 5.05; N, 14.64.

Examples 23 and 24 were prepared in a manner similar to Example 22.

### Example 23

### 4-(2-Methoxy-phenyl)-2-piperazin-1-yl-quinazoline hydrochloride

The identity and purity was confirmed by HPLC/MS. Phenomenex Develosil Combi RP3 50×4.6 mm column, 45° C., 98-2% H₂O (in CH₃CN), hold 0.5 min, run time 4 min. APCI MS m/z 321 (M⁺+1, 100%) 1.99 min

### Example 24

### 4-(2-Methyl-phenyl)-2-(piperazin-1-yl)-quinazoline hydrochloride

The identity and purity was confirmed by HPLC/MS. Phenomenex Develosil Combi RP3 50×4.6 mm column, 45° C., 98-2% H₂O (in CH₃CN), hold 0.5 min, run time 4 min. APCI MS m/z 305 (M⁺+1, 100%) 1.78 min

### Example 25

### 4-(4-Fluoro-phenyl)-2-(4-methyl-piperazin-1-yl)-quinazoline

### Step A: 4-(4-Fluoro-phenyl)-1H-quinazolin-2-one

Ethyl chloroformate (40 g, 0.369 mole) is added dropwise to a solution of 2-cyanoaniline (40 g, 0.33 mole) in anhydrous pyridine (135 mL) at 0° C. The mixture is stirred at 0° C. for 30 minutes and warmed to room temperature for 2 hours. The reaction is poured into cold water and filtered. The resulting solid is recrystallized from ethyl acetate/cyclohexane to give 56.1 g of (2-cyano-phenyl)-carbamic acid ethyl ester.

To a 1M solution of 4-Fluorophenylmagnesium bromide in THF (tetrahydrofuran) (114 mL, 0.114 mole) at 0° C. is added a solution of (2-cyano-phenyl)-carbamic acid ethyl ester (10.0 g, 0.0526 mole) in THF (50 mL). The reaction mixture is stirred for 1.5 hours at 0° C. and 1.5 hours at room temperature. Saturated ammonium chloride (50 mL) and water (50 mL) are added. The mixture is stirred for several hours, filtered and dried to give 11.9 g of the title compound as a white solid; mp 294-298° C.

### Step B: 2-Chloro-4-(4-fluoro-phenyl)-quinazoline

Prepared in a manner similar to Example 1, Step B; mp 183-184° C.

### Step C: 4-(4-Fluoro-phenyl)-2-(4-methyl-piperazin-1-yl)-quinazoline

Prepared in a manner similar to Example 1, Step C. The product was recrystallized from 95% ethanol to give the title compound as yellow needles; mp 130-131° C. Elemental analysis found for C₁₉H₁₉N₄F: C, 71.19; H, 6.01; N, 17.47.

### Examples 26-41 were prepared in a manner similar to Example 25.

### Example 26

### 4-(3-Fluoro-phenyl)-2-(4-methyl-piperazin-1-yl)-quinazoline

Mp 134-135° C.; Elemental analysis found for C₁₉H₁₉N₄F: C, 70.92; H, 5.94; N, 17.57; F, 5.75.

### Example 27

### 2-(4-Methyl-piperazin-1-yl)-4-thiophen-2-yl-quinazoline oxalate

Mp 216-217° C.; Elemental analysis found for C₁₇H₁₈N₄S×C₂H₂O₄: C, 56.62; H, 5.06; N, 13.64; S, 8.72.

### Example 28

### 4-Benzyl-2-piperazin-1-yl-quinazoline hydrochloride

Identity and purity confirmed by HPLC/MS. Phenomenex Develosil Combi RP3 50×4.6mm column, 45° C., 98-2% H₂O (in CH₃CN), hold 0.5 min, run time 4 min. 1.97 min, APCI MS m/z 305 (M⁺+1, 100%).

### Example 29

### 4-(2,6-Difluoro-phenyl)-2-piperazin-4-yl-quinazoline hydrochloride

Mp 295-297° C.; Elemental analysis found for C₁₈H₁₆F₄N₄.HCl: C, 59.76; H, 4.70; N, 14.96; Cl, 9.56; F, 10.23.

### Example 30

### (R)-(−)-2-(2-Methyl-piperazin-1-yl)-4-phenyl-quinazoline hydrochloride

Mp 150-161° C.; Elemental analysis found for C₁₉H₂₀N₄.1 HCl.0.2 H₂O: C, 65.39; H, 6.21; N, 16.01; Cl, 11.11.

### Example 31

### (R)-(+)-2-(3-Methyl-piperazin-1-yl)-4-phenyl-quinazoline

Mp 241-242° C.; Elemental analysis found for C₁₉H₂₀N₄.1.05 HCl.0.2 H₂O: C, 66.09; H, 6.26; N, 16.08; Cl, 10.50.

### Example 32

### 2-(3,9-Diaza-bicyclo[3.3.1]non-3-yl)-4-phenyl-quinazoline hydrochloride

Mp>300° C.; Elemental analysis found for C₂₁H₂₂N₄.HCl.0.05 H₂O: C, 68.31; H, 6.42; N, 14.95; Cl, 9.53.

### Example 33

### 2-(3,9-Diaza-bicyclo[3.3.1]non-3-yl)-4-(2-fluoro-phenyl-quinazoline hydrochloride

Mp 282-290° C.; Elemental analysis found for C₂₁H₂₁FN₄.HCl.0.4 H₂O: C, 63.94; H, 5.50; N, 14.10; F, 4.67; Cl, 9.03.

### Example 34

### (S)-(+)-1-[4-(2-Fluoro-phenyl)-quinazolin-2-yl]-pyrrolidin-3-ylamine hydrochloride

Mp 285-287° C.; Elemental analysis found for C₁₈H₁₇FN₄.1.05 HCl: C, 62.22; H, 5.07; N, 16.01; F, 5.46.

### Example 35

### (S)-(+)-1-[4-(2-Fluoro-phenyl)-quinazolin-2-yl]-pyrrolidin-3-yl}-methyl-amine hydrochloride

Mp 257-258° C.; Elemental analysis found for C₁₉H₁₉FN₄.HCl: C, 63.48; H, 5.72; N, 15.36; Cl, 10.07; F, 5.29.

### Example 36

### 4-(2-Chloro-6-fluoro-phenyl)-2-piperazin-1-yl-quinazoline hydrochloride

Mp 266-268° C.

### Example 37

### 4-(2,3-Difluoro-phenyl)-2-piperazin-1-yl-quinazoline hydrochloride

Mp 273-276° C.

### Example 38

### 4-(2,4-Difluoro-phenyl)-2-piperazin-1-yl)-quinazoline hydrochloride

Mp 261-263° C.

### Example 39

### 4-(2-Fluoro-phenyl)-2-(hexahydro-pyrrolo[3,4-c]pyrrol-2-yl)-quinazoline hydrochloride

Mp 270-278° C.; Elemental analysis found for C₂₀H₁₉FN₄.HCl.0.5 H₂O: C, 63.33; H, 5.48; N, 14.52; F, 4.98; Cl, 9.49.

### Example 40

### (S)-(+)-1-[4-(2-Fluoro-phenyl)-quinazolin-2-yl]-piperidin-3-ylamine hydrochloride

Mp 288-289° C.; Elemental analysis found for C₁₉H₁₉FN₄.HCl: C, 63.32; H, 5.38; N, 15.38; Cl, 10.11; F, 5.30.

### Example 41

### 4-(2-Fluoro-phenyl)-2-(piperidin-4-yl)-quinazoline hydrochloride

### Step A: 1-Methyl-piperidine-4-carboxylic acid (2-bromo-phenyl)-amide

Oxalyl chloride (58 mL, 116 mmol) was added to a mixture of 1-methyl-piperidine-4-carboxylic acid hydrochloride (10.44 g, 58 mmol) in CH₂Cl₂ (50 mL) followed by a catalytic amount of DMF (dimethylformamide) (gas evolved). The mixture was stirred at ambient temperature for 3 hrs. Solvent was co-evaporated with heptane. The resulting white solid was suspended in CH₂Cl₂ (100 mL), cooled in ice bath and a solution of 2-bromoaniline (10 g, 58 mmol) in CH₂Cl₂ (10 mL) was added slowly, followed by addition of triethylamine (24.3 mL, 174 mmol). The resulting white suspension was stirred at ambient temperature overnight. A solution of IN NaOH was added to the mixture and stirred until all solid dissolved. The phases were separated and aqueous phase was extracted with CH₂Cl₂. Organics were combined and washed with saturated NaHCO₃ solution, brine, dried over MgSO₄, and filtered. Evaporation of solvent gave off-white solid, which was stirred in t-butylmethylether (80 mL) for 15 min. Solid was filtered, and dried at 45° C. vacuum oven over night to yield 1-methyl-piperidine-4-carboxylic acid (2-bromo-phenyl)-amide as a white solid 11.72 g. APCI MS m/z 297 (M⁺+1, 100%), 299.

### Step B: 4-(2-Fluoro-phenyl)-2-(1-methyl-piperidin-4-yl)-quinazoline

n-Butyllithium (58 ml of a 2.35 M solution in isopar, 135 mmol) was slowly added to a suspension of 1-methyl-piperidine-4-carboxylic acid (2-bromo-phenyl)-amide (20.0 g, 67 mmol) in Et₂O (200 ml) at −78° C. Reaction mixture was stirred at −78° C. for 1 h, and then warmed to 40° C. for 2 h. The reaction mixture was cooled to −78° C. and 2-fluorobenzonitrile (7.6 ml, 70 mmol) was added. The resulting orange solution was stirred at −78° C. for 2 h then allowed to warm slowly to room temperature overnight. The reaction mixture was then quenched with saturated ammonium chloride (50 ml). Ethyl acetate (200 ml) and 1N NaOH solution (50 ml) was added and the phases were separated. The organic layers were dried over magnesium sulfate, filtered and solvents removed under reduced pressure to yield crude product as an orange oil. The residue was purified by chromatography using silica gel and 100% ethyl acetate to 95:5 ethyl acetate/triethylamine to yield 12.1 g of 4-(2-fluoro-phenyl)-2-(1-methyl-piperidin-4-yl)-quinazoline as a waxy off-white solid. APCI MS m/z 322 (M⁺+1, 100%)

### Step C: 4-(2-Fluoro-phenyl)-2-(piperidin-4-yl)-quinazoline hydrochloride

4-(2-Fluoro-phenyl)-2-(piperidin-4-yl)-quinazoline hydrochloride was prepared from 4-(2-fluoro-phenyl)-2-(1-methyl-piperidin-4-yl)-quinazoline in a manner similar to the procedure provided in step E of Example 47. Elemental analysis found for C₁₉H₁₈F₁N₃.H₁Cl₁.0.1 H₂O: C, 65.93; H, 5.52; N, 11.94.

Examples 42-46 were prepared according to Example 41.

### Example 42

### 4-Phenyl-2-piperidin-4-yl-quinazoline hydrochloride

Elemental analysis found for C₁₉H₁₉N₃: C, 69.14; H, 6.11; N, 12.66.

### Example 43

### 4-(2-Fluoro-phenyl)-2-(1-methyl-piperidin-4-yl)-quinazoline

Elemental analysis found for C₂₀H₂₀F₁N₃: C, 74.47; H, 6.31; N, 12.93.

### Example 44

### 4-(2-Chloro-phenyl)-2-(1-methyl-piperidin-4-yl)-quinazoline

Elemental analysis found for C₂₀H₂₀Cl₁N₃.0.2H₂O: C, 70.29; H, 5.90; N, 12.18.

### Example 45

### 4-(2-Chloro-phenyl)-2-piperidin-4-yl-quinazoline hydrochloride

Elemental analysis found for C₂₀H₂₁N₃.H₁Cl₁.H₁Cl: C, 63.16; H, 5.37; N, 11.60.

### Example 46

### 4-(2-Methoxy-phenyl)-2-piperidin-4-yl-quinazoline hydrochloride

Elemental analysis found for C₂₀H₂₁N₃O1.H₁Cl₁.0.75 H₂O.0.3 C₄H₈O₃: C, 64.05; H, 6.40; N, 10.55.

### Example 47

### 4-(2-Methyl-phenyl)-2-(piperidin-4-yl)-quinazoline hydrochloride

### Step A: Pyridin-4-yl-4-o-tolyl-quinazoline

Anthranilonitrile (1.18 g, 10 mmol) in 10 mL Et₂O was added slowly to a solution of o-tolylmagnesium bromide (20 mmol) in 20 mL Et₂O. The reaction was refluxed for 2 h then cooled to 0° C. and isonicotinoyl chloride (2.1 g, 15 mmol) was added portionwise. The reaction was stirred at 0° C. for 15 minutes and then warmed to reflux for 1 hour. The reaction was cooled to ambient temperature and saturated KH₂PO₄ solution (50 mL) and Et₂O (40 mL) were added. The phases were separated and the collected organic layers was washed twice with saturated KH₂PO₄ solution and then once with brine. The organic layer was then dried over MgSO₄, filtered and concentrated to yield a yellow solid. Recrystallization of the solids in EtOH yielded 591 mg of desired product. The remaining material was purified by chromatography on silica gel using 2:1 ethyl acetate/hexanes to yield 1.87 g of 2-Pyridin-4-yl-4-o-tolyl-quinazoline as a light yellow solid. APCI MS m/z 298 (M⁺+1, 100%)

### Step B: 1-Methyl-4-(4-o-tolyl-quinazolin-2-yl)-pyridinium iodide

Iodomethane (5 mL, 8.0 mmol) was added to a solution of 2-Pyridin-4-yl-4-o-tolyl-quinazoline (2.15 g, 7.2 mmol) in acetonitrile (60 mL). The reaction mixture gently refluxed 4 h then heated at 40° C. overnight. A precipitate formed. Solvents removed under reduced pressure and resulting solids washed with Et₂O and collected by filtration and vacuumed dried to obtain 3.13 g of 1-methyl-4-(4-o-tolyl-quinazolin-2-yl)-pyridinium iodide as a bright yellow solid APCI MS m/z 298 (M⁺—CH₃, 100%), 313 (M⁺+1)

### Step C: 2-(1-Methyl-1,2,3,6-tetrahydro-pyridin-4-yl)-4-o-tolyl-quinazoline

Sodium borohydride (1.29 g, 34 mmol.) was added to a solution of 1-methyl-4-(4-o-tolyl-quinazolin-2-yl)-pyridinium iodide (3.00 g, 6.8 mmol.), in methanol (15 mL), cooled to 0° C. The reaction is exothermic and a gas evolves. The reaction mixture was stirred at 0° C. for 0.5 hours, and then at ambient temperature overnight. The reaction was cooled to 0° C. and quenched with 25 mL 6 N HCl solution. The solvents removed under reduced pressure and remaining aqueous phase cooled to 0° C. and pH adjusted to 10-11 with saturated NH₄OH, then extracted three times with 100 mL EtOAc. The organic layers were combined and washed with brine, dried over MgSO₄, filtered and solvents removed to yield 2.15 g orange solid. Recrystallization in 95% ethanol yielded 1.487 g of 2-(1-methyl-1,2,3,6-tetrahydro-pyridin-4-yl)-4-o-tolyl-quinazoline as a gray solid. APCI MS m/z 298, 316 (M⁺+1, 100%)

### Step D: 2-(1-Methyl-piperidin-4-yl)-4-o-tolyl-quinazoline

2-(1-Methyl-1,2,3,6-tetrahydro-pyridin-4-yl)-4-o-tolyl-quinazoline was reduced using 10% Pd(OH)₂/C in THF. The solvent was removed under reduced pressure to yield 1.01 g. The residue was chromatographed on silica gel column using 95:5 ethylacetate/triethylamine as eluent. The desired product was obtained as a light yellow solid (380 mg). APCI MS m/z 318 (M⁺+1, 100%)

### Step E: 2-Piperidin-4-yl-4-o-tolyl-quinazoline hydrochloride

1-Chloroethylchloroformate (520 μL, 4.73 mmol) was added to a solution of 2-(1-methyl-piperidin-4-yl)-4-o-tolyl-quinazoline (300 mg, 0.95 mmol) and proton sponge (1,8-bis(dimethylamino)naphthalene) (111 mg, 0.52 mmol) in 10 ml of methylene chloride. The reaction was refluxed for 1.5 hours. The intermediate carbamate was purified by chromatographing on silica gel using 1:2 ethylacetate/hexanes as eluent. The resulting light yellow oil was dissolved in methanol (7 mL) and warmed to 60° C. for 1.5 hours. The solvent was removed under reduced pressure to yield an off-white solid that was triturated with ethyl acetate, collected by filtration and dried in a 50° C. vacuum oven to yield 235 mg of the title compound as a white powder. Elemental analysis found for C₂₀H₂₁N₃.H₁Cl₁.0.1 H₂O: C, 70.22; H, 6.53; N, 12.09.

### Example 48

### 4-Phenyl-2-piperidin-3-yl-quinazoline hydrochloride

### Step A: 2-(1-Methyl-piperidin-4-yl)-4-phenyl-quinazoline

1-Methyl-piperidine-4-carboxylic acid (2-cyano-phenyl)-amide (10.0 g, 0.0412 M) dissolved in dry ether was added dropwise to a well-stirred solution of phenylmagnesium bromide (1M, 0.09 M) in ether at reflux. After the addition was completed, the mixture was refluxed for an additional 4 hours, cooled and then treated with 10% aqueous ammonium chloride and ether. The ether phase was washed with water, dried over sodium sulfate, filtered and evaporated in vacuo to give 16.5 g of a tan semisolid. This material was chromatographed (silica gel, 1:10:89 NH₄OH:MeOH:CH₂Cl₂) to give a solid. Recrystallization was carried out from hexane, mp 83-86° C.; Elemental analysis found for C₂₀H₂₁N₃: C, 79.25; H, 7.14; N, 13.88.

### Step B: 4-Phenyl-2-piperidin-3-yl-quinazoline hydrochloride

4-Phenyl-2-piperidin-3-yl-quinazoline hydrochloride was prepared from 2-(1-methyl-piperidin-4-yl)-4-phenyl-quinazoline in a manner similar to the procedure provided in step E of Example 47. Mp 231-232° C.; Elemental analysis found for C₁₉H₁₉N₃.HCl.0.2 H₂O: C, 69.24; H, 6.30; N, 12.72; Cl, 10.83.

### Example 49

### 4-Phenyl-2-piperidin-3-yl-quinazoline hydrochloride

### Step A: 1-tert-Butyl 3-methyl 3-(4-phenylquinazolin-2-yl)-piperidine-1,3-dicarboxylate

To a solution of LDA (lithium diisopropylamide) (prepared by mixing 0.64 mL of i-Pr₂NH and 2.7 mL of a 1.6 M solution of n-BuLi in hexanes in 4 mL of anhydrous THF) at −78° C. under N₂ was added a solution of 1-(1,1-dimethylethyl) 3-methyl 1,3-piperidinedicarboxylate (prepared as in U.S. Pat. No. 5,190,953, 1.009 g, 4.15 mmol) in 6 mL of anhydrous THF. The reaction mixture was stirred at −78° C. for 30 min. A solution of 2-Chloro-4-phenylquinazoline (1.002 g, 4.16 mmol) in 8 mL of anhydrous THF was then added dropwise into the reaction mixture at −78° C. The reaction mixture was stirred at −78° C. for 20 min. and then slowly warmed to room temperature in 3 hours. The mixture was cooled back to 0° C. Saturated NH₄Cl solution was added to quench the reaction. The mixture was extracted with EtOAc (2×50 mL). The combined organic extracts was dried with MgSO₄, filtered and concentrated. The residue was chromatographed on silica gel with 10% ethyl acetate in hexanes to remove the unreacted 2-Chloro-4-phenylquinazoline. The column was then eluted with 20% ethyl acetate in hexanes to give 1.803 g of product as a white foam. APCI MS m/z 348, 392, 448 (M⁺+1, 100%).

### Step B: tert-Butyl-3-(4-phenylquinazolin-2-yl)-piperidine-1-carboxylate

A mixture of 1-tert-butyl-3-methyl-3-(4-phenylquinazolin-2-yl)-piperidine-1,3-dicarboxylate (0.766 g, 1.734 mmol) and sodium cyanide (0.425 g, 8.672 mmol) in 3 mL of DMF was refluxed for 72 hours. The reaction mixture was cooled to RT. H₂O (50 mL) and EtOAc (50 mL) were added. The mixture was stirred at room temperature for 10 min. The organic layer was collected and the aqueous layer was extracted with EtOAc (50 mL). The combined organic layers were washed with saturated NaCl solution (1×5 mL). The organic layer was dried with MgSO₄, filtered and concentrated. The residue was chromatographed on silica gel with 20% EtOAc in hexanes to give 0.259 g of product as a white foam. APCI MS m/z 290, 390 (M⁺+1, 100%).

### Step C: 4-Phenyl-2-piperidin-3-yl-quinazoline

A mixture of tert-butyl 3-(4-phenylquinazolin-2-yl)-piperidine-1-carboxylate (0.259 g, 0.665 mmol) in 0.2 mL water and 2 mL of TFA was stirred at room temperature for 1 hour. The mixture was loaded onto a 5% HOAc in MeOH pre-washed Varian Mega bond elut SCX column. The column was washed with MeOH (3×30 mL) to remove TFA. It was then eluted with 1N NH₃ in MeOH (2×40 mL) to give 0.161 g of product as a light yellow oil. APCI MS m/z 290 (M⁺+1, 100%).

### Step D: 4-Phenyl-2-piperidin-3-yl-quinazoline hydrochloride

To a solution of 4-phenyl-2-piperidin-3-ylquinazoline (0.161 g, 0.556 mmol) in 30 mL of THF was added a 1.0 M HCl solution (0.56 mL, 0.56 mmol) in ether. The yellow solution turned into a light pink suspension. The mixture was stirred at ambient temperature for 15 min. The solid was collected by filtration and washed with Et₂O (2×5 mL). The solid was dried in vacuum at 90° C. overnight to give 0.163 g of the title compound as a pale pink solid. Mp=231-232° C. Elemental analysis found for C₁₉H₁₉N₃.HCl.0.2 H₂O: C, 69.24; H, 6.30; N, 12.72; Cl, 10.83.

### Methyl 3-(4-phenylquinazolin-2-yl)-piperidine-3-carboxylate

A mixture of 1-tert-butyl 3-methyl 3-(4-phenylquinazolin-2-yl)-piperidine-1,3-dicarboxylate (1.027 g, 2.295 mmol) in 5.5 mL of TFA and 0.55 mL of water was stirred at room temperature for 1 hour. The mixture was loaded onto a 5% HOAc in MeOH pre-washed Varian Mega bond elut SCX column. The column was washed with MeOH (3×60 mL) to remove TFA and then eluted with 1N NH₃ in MeOH (3×50 mL) to give 0.620 g of product as a colorless oil. APCI MS m/z 348 (M⁺+1, 100%).

### Methyl 3-(4-phenylquinazolin-2-yl)-piperidine-3-carboxylate hydrochloride

To a solution of methyl 3-(4-phenylquinazolin-2-yl)-piperidine-3-carboxylate (0.620 g, 1.785 mmol) in 35 mL of THF was added a 1.0 M solution of HCl in ether. The solution was stirred at room temperature for 15 min and was then concentrated on a rotavap to a volume of approximately 2 mL. A white precipitate was formed. THF (30 mL) was then added and the suspension was stirred at room temperature for 15 min. The solid was collected by filtration and washed with THF (2×5 mL). The solid was dried over weekend under vacuum at 95° C. to give 0.646 g of product as a white solid. mp=143-145° C. (dec., gas evolution).

Examples 50-52 were prepared in accordance with Example 49.

### Example 50

### 4-(4-Phenyl-quinazolin-2-yl)piperidine-4-carboxylic acid methyl ester hydrochloride

Mp 227-228° C.; Elemental analysis found for C₂₁H₂₁N₃O₂: C, 64.01; H, 5.79; N, 10.36; Cl, 9.02.

### Example 51

### 4-[4-(2-Fluoro-phenyl)-quinazolin-2-yl]-piperidine-4-carboxylic acid methyl ester hydrochloride

Mp 210-211° C.; Elemental analysis found for C₂₁H₂₀FN₃O₂.HCl.0.35 H₂O: C, 61.81; H, 5.41; N, 10.11; Cl, 8.60; F, 4.65.

### Example 52

### 3-(4-Phenyl-quinazolin-2-yl)-piperidine-3-carboxylic acid methyl ester hydrochloride

Mp 143-145° C.; Elemental analysis found for C₂₁H₂₁N₃O₂.HCl.0.1 H₂O: C, 65.53; H, 6.21; N, 10.31; Cl, 8.96.

### Example 53

### 2-Piperazin-1-yl-4-s-tolyl-quinazoline hydrochloride

The titled compound was made in a manner similar to Example 49 and was verified via LC/MS.

Examples 54-56 were made in a manner similar to Example 1.

### Example 54

### 2-(3-Methyl-piperazin-1-yl)-4-phenyl-quinazoline

MP 107-108° C.

### Example 55

### 2-(3,9-Diaza-bicyclo[3.3.1]non-9-yl)-4-phenyl-quinazoline

The amine was made in accordance with a procedure in Biagi et al., Farmaco (2000) 55(8), 551, 553. MP 247-246.

### Example 56

### 2-(3,8-Diaza-bicyclo[3.2.1]oct-8-yl)-4-phenyl-quinazoline

MP 278-280; Elemental analysis found for C₂₀ H₂₀ N₄.1.15 HCl.0.35 H₂O: C 65.89, H 5.99, N, 14.98, Cl 10.89.

### Example 57

### 2-[1,4]Diazepan-1-yl-4-(2,3-difluoro-phenyl)-quinazoline

The titled compound was made in accordance with Example 18. MP 203-206; Elemental analysis found for C₁₉H₁₈F₂N₄:

### Example 58

### 4-(2,6-Difluoro-phenyl)-quinazolin-2-yl]-pyrrolidin-3-yl-amine

S-[4-(2,6-Difluoro-phenyl)-quinazolin-2-yl]-pyrrolidin-3-yl-amine was prepared as in Example 140, with the exception that (S)-1-Cbz-3-aminopyrrolidine was used instead of 1-methyl-piperazine, and the benzyloxycarbonyl (Cbz) group was removed using palladium on carbon: (S)-1-Cbz-3-aminopyrrolidine (840 mg, 3.8 mmol) was added to a suspension of 2-chloro-4-(2,6-difluoro-phenyl)-quinazoline (500 mg, 1.81 mmol) in toluene (10 mL). The reaction mixture stirred at reflux for 16 hours. Silica gel chromatography of the reaction mixture (0-40% ethyl acetate in hexanes) afforded 0.8 g (96%) of 3-[4-(2,6-Difluoro-phenyl)-quinazolin-2-ylamino]-pyrrolidine-1-carboxylic acid benzyl ester as a yellow oil.

3-[4-(2,6-Difluoro-phenyl)-quinazolin-2-ylamino]-pyrrolidine-1-carboxylic acid benzyl ester (0.8 g, 1.7 mmol) and 20% Pd on C (0.092 g, 0.17 mmol Pd) in a flask and purged with N2. Added 20 mL MeOH. Flask was purged with H2 (10×). Reaction mixture was stirred at room temperature for 30 min. Reaction was diluted with ethyl acetate (20 mL) and filtered through celite. The solvent was removed in vacuo and the resulting residue was dissolved in ethyl acetate (10 mL). To this solution was added 2M HCl in diethyl ether (2.0 mL). The resulting solid was collected by filtration and dried in a 45° C. vacuum oven to yield 0.553 mg of the title compound as an off-white powder. Elemental analysis found for C₁₈H₁₆F₂N₄:

### Example 59

### 7-Fluoro-4-(2-fluoro-phenyl)-2-piperazin-1-yl-quinazoline

The titled compound was made in accordance with Example 18. MP 147-148.

### Example 60

### 4-(3-Fluoro-phenyl)-2-piperazin-1-yl-quinazoline

The titled compound was made in accordance with Example 22. Elemental analysis found for C₁₈H₁₇FN₄.HCl.0.1H₂O: C 62.06, H 5.11, N 15.90.

### Example 61

### 4-(2-Chloro-4-fluoro-phenyl)-2-piperazin-1-yl-quinazoline

The titled compound was made in accordance with Example 18. MP 248-251; Elemental analysis found for C₁₈H₁₆ClFN₄.1.05 HCl.0.15 H₂O.0.15 THF: C 56.59, H 4.59, N 14.14, F 4.77, Cl 18.12.

### Example 62

### 4-(4-Chloro-phenyl)-2-piperazin-1-yl-quinazoline

The titled compound was made in accordance with Example 22. Elemental analysis found for C₁₈H₁₇ClN₄.HCl: C 59.54, H 4.81, N 15.18.

### Example 63

### 4-(2,6-Dichloro-phenyl)-2-piperazin-1-yl-quinazoline

The titled compound was made in accordance with Example 18. MP>300.

Examples 64 and 65 were made in a manner similar to Example 41.

### Example 64

### 6-Fluoro-4-(2-fluoro-phenyl)-2-piperidin-4-yl-quinazoline

Elemental analysis found for C₁₉H₁₇F₂N₃.HCl: C 62.86, H 4.87, N 4.87.

### Example 65

### 7-Fluoro-4-(2-fluoro-phenyl)-2-piperidin-4-yl-quinazoline

Elemental analysis found for C₁₉H₁₇F₂N₃.HCl; C 62.69, H 4.80, N 11.60.

Examples 66-68 were made in a manner similar to Example 48.

### Example 66

### 4-(3-Fluoro-phenyl)-2-piperidin-4-yl-quinazoline

Elemental analysis found for C₁₉H₁₈FN₃.HCl: C 66.07, H 5.27, N 11.84.

### Example 67

### 4-(3-Fluoro-phenyl)-2-(1-methyl-piperidin-4-yl)-quinazoline

Elemental analysis found for C₂₀H₂₀FN₃.1.50 HCl: C 63.51, H 5.74, N 10.86.

### Example 68

### 4-(4-Fluoro-phenyl)-2-piperidin-4-yl-quinazoline

Elemental analysis found for C₁₉H₁₈FN₃.HCl: C 66.24, H 5.60, N 12.15.

Examples 69-71 were made in a manner similar to Example 41.

### Example 69

### 4-(2,6-Difluoro-phenyl)-2-(1-methyl-piperidin-4-yl)-quinazoline

Elemental analysis found for C₂₀H₁₉F₂N₃.0.10 H₂O: C 70.19, H 5.45, N 12.19.

### Example 70

### 4-(2,6-Difluoro-phenyl)-2-piperidin-4-yl-quinazoline

Elemental analysis found for C₁₉H₁₇F₂N₃.HCl: C 62.74, H 4.90, N 11.47.

### Example 71

### 4-(2,3-Difluoro-phenyl)-2-piperidin-4-yl-quinazoline

Elemental analysis found for C₁₉H₁₇F₂N₃.HCl.0.10 H₂O: C 62.51, H 4.80, N 11.38.

### Example 72

### 4-(2,4-Difluoro-phenyl)-2-piperidin-4-yl-quinazoline

The titled compound was made in accordance with Example 47. Elemental analysis found for C₁₉H₁₇F₂N₃.HCl: C 62.80, H 5.00, N 11.46.

Examples 73-75 were made in a manner similar to Example 41.

### Example 73

### 2-Piperidin-4-yl-4-(2,3,6-trifluoro-phenyl)-quinazoline

Elemental analysis found for C₁₉H₁₆F₃N₃.HCl: C 59.77, H 4.52, N 10.66.

### Example 74

### 4-(2-Chloro-6-fluoro-phenyl)-2-(1-methyl-piperidin-4-yl)-quinazoline

Elemental analysis found for C₂₀H₁₉ClFN₃: C 67.37, H 5.25, N 11.64.

### Example 75

### 4-(2-Chloro-6-fluoro-phenyl)-2-piperidin-4-yl-quinazoline

Elemental analysis found for C₁₉H₁₇ClFN₃.HCl: C 60.34, H 4.62, N 10.97.

### Example 76

### 2-Piperidin-4-yl-4-o-tolyl-quinazoline

Example 76 was made in accordance with Example 47. Elemental analysis found for C₂₀H₂₁N₃.HCl.0.10 H₂O: C 70.22, H 6.53, N 12.09.

Examples 77 and 78 were made in a manner similar to Example 41.

### Example 77

### 4-(2-Fluoro-phenyl)-2-piperidin-3-yl-quinazoline

Elemental analysis found for C₁₉H₁₈FN₃.HCL.0.25 H₂O: C 65.26, H 5.32, N 11.82.

### Example 78

### 2-Piperidin-3-yl-4-o-tolyl-quinazoline

Elemental analysis found for C₂₀H₂₁N₃.HCl.0.30 H₂O: C 69.22, H 6.61, N 12.08.

### Example 79

### 4-(2-Fluoro-phenyl)-2-(4-phenyl-piperidin-4-yl)-quinazoline

### Step A: 4-[2-(2-Fluoro-benzoyl)-phenylcarbamoyl]-4-phenyl-piperidine-1-carboxylic acid tert-butyl ester

To a mixture of the acid in pyridine and methylene chloride at room temperature was added thionyl chloride dropwise and the mixture allowed to stir for one hour. The reaction was concentrated in vacuo to give a solid which was treated with pyridine, a catalytic amount of dimethylaminopyridine and 2-amino-2′-fluorobenzophenone and the solution was heated to 50C overnight. The solution was concentrated in vacuo, dissolved in EtOAc, washed with IN HCl, saturated potassium carbonate, brine, dried over magnesium sulfate, filtered and concentrated in vacuo to give a yellow solid.

### Step B: 4-[4-(2-Fluoro-phenyl)-quinazolin-2-yl]-4-phenyl-piperidine-1-carboxylic acid tert-butyl ester

The ketoamide was dissolved in NH3/EtOH and heated to 110C for 30 hours. The solution was concentrated in vacuo. This material was chromatographed (10-20% EtOAc) to give an off white solid.

Step C

To a solution of the BOC-amine in 10 ml of CH₂Cl₂ was added 10 ml of TFA and the solution allowed to stir for 1 h. The solution was conc. in vacuo, suspended in EtOAc, washed with sat K₂CO₃ (2×), dried over sodium sulfate, filtrated and concentrated. The resultant semisolid was dissolved in EtOAc and 2.5 ml of 1M HCl was added and the mixture further diluted with Et2O. The resultant off-white solid was collected by filtration and dried (0.84 g). This material was 92-95% pure by HPLC. This was washed with EtOAc to give material that was 100% pure by HPLC/MS.

Examples 80 and 81 were prepared in a manner similar to Example 1.

### Example 80

### 2-(2,5-Diaza-bicyclo[2.2.1]hept-2-yl)-4-phenyl-quinazoline

### Example 81

### 2-(Hexahydro-pyrrolo[3,4-c]pyrrol-2-yl)-4-phenyl-quinazoline

MP 267-270; Elemental analysis found for C₂₀H₂₀N₄.HCl.0.55 H₂O: C 65.87, H 6.20, N 15.20, Cl 9.73.

### Example 82

### S-4-(2,4-Difluoro-phenyl)-2-(2-methyl-piperazin-1-yl)-quinazoline

The titled compound was prepared in a manner similar to Example 139. MS (APCI) M+1=341.2; 1H NMR (400 MHz, DMSO-D6) d ppm 1.21 (d, J=6.83 Hz, 3H), 2.56 (m, 1H), 2.79 (d, J=2.68 Hz, 2H), 2.95 (d, J=12.20 Hz, 1H), 3.04 (m, 1H), 4.47 (d, J=13.42 Hz, 1H), 4.84 (m, 1H), 7.18 (m, 1H), 7.29 (m, 1H), 7.44 (m, 2H), 7.54 (d, J=8.30 Hz, 1H), 7.68 (m, J=2.00 Hz).

### Example 83

### 4-(2,6-Difluoro-phenyl)-7-fluoro-2-(4-methyl-piperazin-1-yl)-quinazoline

The titled compound was prepared in a manner similar to Example 140. MS (APCI) M+1=359.2; 1H NMR (400 MHz, CHLOROFORM-D) d ppm 2.4 (s, 3 H) 2.5 (m, 4 H) 4.0 (m, 4 H) 6.9 (m, 1 H) 7.1 (m, 2 H) 7.2 (m, 1 H) 7.4 (m, 1 H) 7.5 (m, 1 H).

### Example 84

### R-[4-(2,6-Difluoro-phenyl)-7-fluoro-quinazolin-2-yl]-pyrrolidin-3-yl-amine

The titled compound was prepared in a manner similar to Example 58. MS (APCI) M+1=345.2; 1H NMR (400 MHz, METHANOL-D4) d ppm 2.3 (td, J=13.4, 6.4 Hz, 1 H) 2.5 (td, J=14.5, 7.4 Hz, 1 H) 3.5 (m, 2 H) 3.6 (dt, J=11.7, 7.6 Hz, 1 H) 3.7 (dd, J=12.3, 6.8 Hz, 1 H) 7.2 (t, J=8.5 Hz, 3 H) 7.5 (dd, J=9.8, 2.4 Hz, 1 H) 7.7 (m,2 H).

Examples 85-89 were pared in a manner similar to Example 139.

### Example 85

### S-4-(2-Chloro-6-fluoro-phenyl)-2-(2-methyl-piperazin-1-yl)-quinazoline

MS (APCI) M+1=357.1; 1H NMR (400 MHz, DMSO-D6) d ppm 1.21 (d, J=6.10 Hz, 3H), 2.61 (m, 1H), 2.82 (m, 2H), 3.03 (m, 2H), 4.45 (m, 1H), 4.81 (m, 1H), 7.20 (m, 2H), 7.46 (t, J=8.66 Hz, 1H), 7.56 (t, J=9.03 Hz, 2H), 7.65 (m, 1H), 7.73 (m, 1H).

### Example 86

### R-4-(2,6-Difluoro-phenyl)-2-(2-methyl-piperazin-1-yl)-quinazoline

MS (APCI) M+1=341.1; 1H NMR (400 MHz, DMSO-D6) d ppm 1.21 (d, J=6.59 Hz, 3H), 2.60 (m, 1H), 2.81 (m, 2H), 2.98 (d, J=12.44 Hz, 1H), 3.05 (m, 1H), 3.27 (bs, 2H), 4.45 (dd, J=13.05, 1.59 Hz, 1H), 4.83 (m, 1H), 7.19 (m, 1H), 7.33 (t, J=8.42 Hz, 3H), 7.57 (d, J=8.54 Hz, 1H), 7.70 (m, 2H).

### Example 87

### R-4-(2,3-Difluoro-phenyl)-2-(2-methyl-piperazin-1-yl)-quinazoline

MS (APCI) M+1=341.2; 1H NMR (400 MHz, CHLOROFORM-D) d ppm 1.33 (d, J=6.83 Hz, 3H), 2.27 (bs, 2H), 2.85 (m, 1H), 2.95 (d, J=12.20 Hz, 1H), 3.07 (m, 1H), 3.14 (m, 1H), 3.22 (m, 1H), 4.70 (dd, J=13.42, 2.20 Hz, 1H), 5.08 (m, 1H), 7.14 (m, 1H), 7.29 (m, 3H), 7.49 (m, 1H), 7.64 (m, 2H).

### Example 88

### S-4-(2,3-Difluoro-phenyl)-2-(2-methyl-piperazin-1-yl)-quinazoline

MS (APCI) M+1=341.2; 1H NMR (400 MHz, DMSO-D6) d ppm 1.22 (d, J=6.59 Hz, 3H), 2.57 (m, 1H), 2.79 (d, J=2.68 Hz, 2H), 2.95 (dd, J=11.47, 2.20Hz, 1H), 3.04 (m, 1H), 4.46 (dd, J=13.18, 1.71 Hz, 1H), 4.83 (m, 1H), 7.19 (m, 1H), 7.42 (m, 3H), 7.56 (d, J=8.54 Hz, 1H), 7.68 (m, 2H).

### Example 89

### 7-Chloro-4-phenyl-2-piperazin-1-yl-quinazoline

MS (APCI) M+1=325.1; 1H NMR (400 MHz, DMSO-D6) d ppm 3.19 (m, 4H), 4.12 (m, 4H), 7.30 (dd, J=8.91, 2.07 Hz, 1H), 7.60 (m, 3H), 7.65 (d, J=1.95 Hz, 1H), 7.73 (m, 2H), 7.80 (d, J=9.27 Hz, 1H), 9.32 (bs, 2H).

Examples 90-92 were prepared in a manner similar to Example 18.

### Example 90

### 4-(3-methoxy-phenyl)-2-piperazin-1-yl-quinazoline

MS (APCI) M+1=321.2; 1H NMR (400 MHz, DMSO-D6) d ppm 3.19 (m, 4H), 3.82 (s, 3H), 4.11 (m, 4H), 7.17 (m, 1H), 7.27 (m, 3H), 7.50 (m, 1H), 7.62 (dd, J=8.54, 1.22 Hz, 1H), 7.79 (m, 2H), 9.22 (bs, 2H).

### Example 91

### 6-Bromo-4-phenyl-2-piperazin-1-yl-quinazoline

MS (APCI) M+1=371.1; 1H NMR (400 MHz, DMSO-D6) d ppm 3.19 (m, 4H), 4.11 (m, 4H), 7.60 (m, 4H), 7.73 (m, 2H), 7.87 (m, 2H), 9.32 (bs, 2H).

### Example 92

### 6-Fluoro-4-phenyl-2-piperazin-1-yl-quinazoline

MS (APCI) M+1=309.2; 1H NMR (400 MHz, DMSO-D6) d ppm 3.19 (m, 4H), 4.10 (m, 4H), 7.47 (dd, J=9.64, 2.07 Hz, 1H), 7.60 (m, 3H), 7.73 (m, 4H), 9.29 (bs, 2H).

Examples 93-96 were prepared in a manner similar to Example 58.

### Example 93

### R-1-[4-(2,6-Difluoro-phenyl)-7-fluoro-quinazolin-2-yl]-pyrrolidin-3-ylamine

MS (APCI) M+1=345.2; 1H NMR (400 MHz, METHANOL-D4) d ppm 2.4 (bs, 1 H) 2.6 (bs, 1 H) 4.1 (m, 3 H) 4.2 (m, 2 H) 7.3 (m, 2 H) 7.3 (m, 1 H) 7.7 (m, 1 H)7.7(m, 1 H)7.8(m, 1 H).

### Example 94

### S-1-[4-(2,6-Difluoro-phenyl)-7-fluoro-quinazolin-2-yl]-pyrrolidin-3-ylamine

MS (APCI) M+1=345.2; 1H NMR (400 MHz, METHANOL-D4) d ppm 2.4 (bs, 1 H) 2.6 (bs, 1 H) 4.1 (m, 3 H) 4.2 (m, 2 H) 7.3 (m, 2 H) 7.3 (m, 1 H) 7.7 (m, 1 H) 7.7 (m, 1 H) 7.8 (m, 1 H).

### Example 95

### R-1-[4-(2,6-Difluoro-phenyl)-quinazolin-2-yl]-pyrrolidin-3-ylamine

MS (APCI) M+1=327.2; 1H NMR (400 MHz, METHANOL-D4) d ppm 2.4 (bs, 1 H) 2.7 (bs, 1 H) 4.1 (m, 1 H) 4.1 (m, 4 H) 4.3 (m, 2 H) 7.3 (m, 3 H) 7.6 (m, 2 H) 7.8 (m, 3 H) 8.1 (m, 3 H).

### Example 96

### S-1-[4-(2,6-Difluoro-phenyl)-quinazolin-2-yl]-pyrrolidin-3-ylamine

MS (APCI) M+1=327.2; 1H NMR (400 MHz, METHANOL-D4) d ppm 2.4 (bs, 1 H) 2.7 (bs, 1 H) 4.1 (m, 1 H) 4.1 (m, 4 H) 4.3 (m, 2 H) 7.3 (m, 3 H) 7.6 (m, 2 H) 7.8 (m, 3 H) 8.1 (m, 3 H).

Examples 97 and 98 were prepared in a manner similar to Example 133.

### Example 97

### 7-Fluoro-4-phenyl-2-piperazin-1-yl-quinazoline

MS (APCI) M+1=309.2; 1H NMR (400 MHz, METHANOL-D4) d ppm 3.5 (m, 4 H) 4.4 (m, 4 H) 7.4 (m, 1 H) 7.6 (m, 2 H) 7.7 (m, 2 H) 7.9 (m, 2 H) 8.2 (dd, J=9.2, 5.7 Hz, 1 H).

### Example 98

### 4-(2,6-Difluoro-phenyl)-7-fluoro-2-piperazin-1-yl-quinazoline

MS (APCI) M+1=345.2; 1H NMR (400 MHz, METHANOL-D4) d ppm 3.4 (m, 4 H) 4.3 (m, 4 H) 7.2 (m, 3 H) 7.6 (dd, J=10.0, 2.4 Hz, 1 H) 7.7 (m, 2 H).

Examples 99-102 were prepared in a manner similar to Example 58.

### Example 99

### R-[4-(2,6-Difluoro-phenyl)-quinazolin-2-yl]-pyrrolidin-3-yl-amine

MS (APCI) M+1=327.2; 1H NMR (400 MHz, METHANOL-D4) d ppm 2.3 (td, J=13.5, 6.2 Hz, 1 H) 2.5 (m, 1 H) 3.5 (m, 2 H) 3.6 (dt, J=11.8, 7.7 Hz, 1 H) 3.7 (dd, J=12.4, 6.8 Hz, 1 H) 4.9 (m, 1 H) 7.3 (t, J=8.5 Hz, 2 H) 7.5 (t, J=7.6 Hz, 1 H) 7.7 (m, 2 H) 7.9 (d, J=8.3 Hz, 1 H) 8.0 (t,

### Example 100

### S-[4-(2,6-Difluoro-phenyl)-7-fluoro-quinazolin-2-yl]-pyrrolidin-3-yl-amine

MS (APCI) M+1=345.2; 1H NMR (400 MHz, METHANOL-D4) d ppm 1.8 (m, 1 H) 2.2 (td, J=13.9, 7.8 Hz, 1 H) 2.9 (m, 2 H) 3.1 (ddd, J=11.3, 8.2, 6.8 Hz, 1 H) 3.2 (dd, J=11.7, 6.3 Hz, 1 H) 4.5 (m, J=7.6, 6.4, 4.6, 4.6 Hz, 1 H) 7.0 (m, 1 H) 7.2 (m, 2 H) 7.3 (dd, J=10.5, 2.2 Hz, 1 H) 7.4

### Example 101

### S-[4-(2,6-Difluoro-phenyl)-quinazolin-2-yl]-pyrrolidin-3-yl-amine

MS (APCI) M+1=327.2; 1H NMR (400 MHz, METHANOL-D4) d ppm 2.3 (td, J=13.5, 6.2 Hz, 1 H) 2.5 (m, 1 H) 3.5 (m, 2 H) 3.6 (dt, J=11.8, 7.7 Hz, 1 H) 3.7 (dd, J=12.4, 6.8 Hz, 1 H) 4.9 (m, 1 H) 7.3 (t, J=8.5 Hz, 2 H) 7.5 (t, J=7.6 Hz, 1 H) 7.7 (m, 2 H) 7.9 (d, J=8.3 Hz, 1 H) 8.0 (t,

### Example 102

### R-1-[4-(2,3-Difluoro-phenyl)-quinazolin-2-yl]-pyrrolidin-3-ylamine

MS (APCI) M+1=327; 1H NMR (400 MHz, DMSO-D6) d ppm 1.7 (m, 1 H) 2.0 (m, 2 H) 3.6 (m, 2 H) 3.7 (dd, J=11.2, 5.6 Hz, 2 H) 7.2 (m, 1 H) 7.4 (m, 3 H) 7.6 (d, J=8.3 Hz, 1 H) 7.7 (m, 2 H).

### Example 103

### 4-(3,4-Difluoro-phenyl)-2-(4-methyl-piperazin-1-yl)-quinazoline

The titled compound was prepared in a manner similar to Example 112. MS (APCI) M+1=341.2; 1H NMR (400 MHz, CDCl3-D) d ppm 2.4 (s, 3 H) 2.6 (br. S., 4 H) 4.0 (br. S., 4 H) 7.2 (ddd, J=8.2, 5.3, 2.8 Hz, 1 H) 7.3 (dt, J=10.0, 8.2 Hz, 1 H) 7.5 (m, 1 H) 7.6 (ddd, J=10.9, 7.7, 2.1 Hz, 1 H) 7.7 (m, 2 H) 7.8 (ddd, J=8.2, 1.1, 1.0 Hz, 1 H).

Examples 104-107 were prepared in a manner similar to Example 139.

### Example 104

### R-4-(2,6-Difluoro-phenyl)-7-fluoro-2-(2-methyl-piperazin-1-yl)-quinazoline

MS (APCI) M+1=359.2; 1H NMR (400 MHz, METHANOL-D4) d ppm 1.4 (d, J=7.1 Hz, 3 H) 3.2 (m, 1 H) 3.4 (m, 2 H) 3.5 (m, 2 H) 5.0 (dd, J=14.3, 4.0 Hz, 1 H) 5.4 (m, 1 H) 7.1 (m, 1 H) 7.2 (t, J=8.4 Hz, 2 H) 7.4 (dd, J=10.2, 2.4 Hz, 1 H) 7.5 (m, J=9.1, 6.1, 1.3, 1.3 Hz, 1 H) 7.6 (m, J=

### Example 105

### R-7-Fluoro-2-(2-methyl-piperazin-1-yl)-4-phenyl-quinazoline

MS (APCI) M+1=323.2; 1H NMR (400 MHz, METHANOL-D4) d ppm 1.5 (d, J=7.1 Hz, 6 H) 3.2 (m, 2 H) 3.4 (m, 4 H) 3.6 (m, 3 H) 3.6 (m, 1 H) 5.1 (dd, J=14.6, 3.2 Hz, 2 H) 5.5 (m, 2 H) 7.2 (td, J=8.8, 2.6 Hz, 2 H) 7.5 (dd, J=10.1, 2.6 Hz, 2 H) 7.6 (m, 6 H) 7.8 (ddd, J=6.2, 1.8, 1.6 Hz,

### Example 106

### S-′7-Fluoro-2-(2-methyl-piperazin-1-yl)-4-phenyl-quinazoline

MS (APCI) M+1=323.2; 1H NMR (400 MHz, METHANOL-D4) d ppm 1.5 (d, J=7.1 Hz, 6 H) 3.2 (m, 2 H) 3.4 (m, 4 H) 3.6 (m, 3 H) 3.6 (m, 1 H) 5.1 (dd, J=14.6, 3.2 Hz, 2 H) 5.5 (m, 2 H) 7.2 (td, J=8.8, 2.6 Hz, 2 H) 7.5 (dd, J=10.1, 2.6 Hz, 2 H) 7.6 (m, 6 H) 7.8 (ddd, J=6.2, 1.8, 1.6 Hz,

### Example 107

### S-4-(2,6-Difluoro-phenyl)-7-fluoro-2-(2-methyl-piperazin-1-yl)-quinazoline

MS (APCI) M+1=359.2; 1H NMR (400 MHz, METHANOL-D4) d ppm 1.4 (d, J=7.1 Hz, 3 H)3.2 (m, 1 H) 3.4 (m, 2 H) 3.5 (m, 2 H) 5.0 (dd, J=14.3, 4.0 Hz, 1 H) 5.4 (m, 1 H) 7.1 (m, 1 H) 7.2 (t, J=8.4 Hz, 2 H) 7.4 (dd, J=10.2, 2.4 Hz, 1 H) 7.5 (m, J=9.1, 6.1, 1.3, 1.3 Hz, 1 H) 7.6 (m, J=

### Example 108

### 4-(3,4-difluoro-phenyl)-2-piperazin-1-yl-quinazoline

The titled compound was prepared in a manner similar to Example 112. MS (APCI) M+1=327.1; Elemental analysis found for C18H16F2N4.2HCl: C, 53.78; H, 4.73; N, 13.21; Cl, 15.26.

1H NMR (400 MHz, CD30D-D4) d ppm 3.5 (m, 4 H) 4.4 (m, 4 H) 7.5 (m, 2 H) 7.7 (m, 1 H) 7.9 (m, 2 H) 8.0 (m, 1 H) 8.1 (d, J=8.3 Hz, 1 H).

Examples 109-111 were prepared in a manner similar to Example 139.

### Example 109

### S-1-[4-(2,3-Difluoro-phenyl)-7-fluoro-quinazolin-2-yl]-pyrrolidin-3-yl-amine

MS (APCI) M+1=345.1; Elemental analysis found for C₁₈H₁₅F₃N₄.2HCl: C, 50.96; H, 4.31; N, 12.82; Cl, 15.14.

1H NMR (400 MHz, DMSO-D6) d ppm 2.2 (br. s., 1 H) 2.3 (br. s., 1 H) 3.7 (m, 1 H) 3.8 (m, 4 H) 7.1 (td, J=8.8, 2.6 Hz, 1 H) 7.4 (m, 3 H) 7.6 (m, 1 H) 7.7 (m, 1 H) 8.4 (s, 2 H).

### Example 110

### R-1-[4-(2,3-Difluoro-phenyl)-7-fluoro-quinazolin-2-yl]-pyrrolidin-3-yl-amine

MS (APCI) M+1=345.1; Elemental analysis found for C₁₈H₁₅F₃N₄.2HCl: C, 51.29; H, 4.26; N, 12.95; Cl, 15.27; 1H NMR (400 MHz, DMSO-D6) d ppm 2.2 (br. s., 1 H) 2.3 (br. s., 1 H) 3.7 (m, 1 H) 3.8 (m, 4 H) 7.1 (td, J=8.8, 2.6 Hz, 1 H) 7.4 (m, 3 H) 7.6 (m, 1 H) 7.7 (m, 1 H) 8.4 (s, 2 H).

### Example 111

### 4-(2,3-Difluoro-phenyl)-7-fluoro-2-piperazin-1-yl-quinazoline

MS (APCI) M+1=345.1; Elemental analysis found for C₁₈H₁₅F₃N₄.2HCl: C, 51.16; H, 4.53; N, 12.85; Cl, 14.43; 1H NMR (400 MHz, DMSO-D6) d ppm 3.2 (br. s., 4 H) 4.1 (m, 4 H) 7.2 (td, J=8.9, 2.7 Hz, 1 H) 7.4 (dd, J=10.6, 2.6 Hz, 1 H) 7.4 (m, 2 H) 7.7 (m, 2 H) 9.5 (br. s., 2 H).

### Example 112

### 4-(3,4-Difluoro-phenyl)-7-fluoro-2-(4-methyl-piperazin-1-yl)-quinazoline

### Step A: 7-Fluoro-1H-quinazoline-2,4-dione

2-Amino-4-fluoro-benzoic acid (5.00 g, 32.2 mmol) in water (180 mL) and glacial acetic acid (3 mL) was warmed to 35° C. and slowly treated with a suspension of sodium cyanate (5.24 g, 80.6 mmol) in water (20 mL). Residual sodium cyanate was washed in with three additional portions of water (10 mL each). The reaction mixture was stirred an additional 30 min, after which sodium hydroxide (35 g, 880 mmol) was slowly added giving a white precipitate. Water (100 mL) was added, the reaction mixture was cooled to 0° C., and acidified to pH=4 with concentrated hydrochloric acid. The white solid was filtered, washed with water and dried in vacuo to afford 4.1 g (71%) of the desired product.

### Step B: 4-Chloro-7-fluoro-2-(4-methyl-piperazin-1-yl)-quinazoline

A solution of 7-fluoro-1H-quinazoline-2,4-dione (4.1 g, 23 mmol), dimethylpiperazine (6.2 mL, 46 mmol), and tripropylamine (8.7 mL, 46 mmol) in dioxane (55 mL), was cooled to 0° C. and treated with phosphorous oxychloride (6.4 mL, 68 mmol). The reaction mixture was heated to 100° C. for 1 h, cooled to ambient temperature and stirred an additional 16 h. Chloroform (about 200 mL) was added and the mixture was slowly poured over ice. After neutralizing to pH>10 with 25% NaOH (about 30 mL), the organic layer was separated. The aqueous layer was extracted again with chloroform, and the combined organic extracts were dried over sodium sulfate, filtered and concentrated. Silica gel chromatography (10% ethyl acetate in hexanes) afforded 3.7 g (58%) of the desired product as a light brown solid.

### Step C: 4-(3,4-Difluoro-phenyl)-7-fluoro-2-(4-methyl-piperazin-1-yl)-quinazoline

4-Chloro-7-fluoro-2-(4-methyl-piperazin-1-yl)-quinazoline (0.400 g, 1.43 mmol), 3,4-difluoroboronic acid (0.270 g, 1.71 mmol), potassium fluoride (0.248 g, 4.27 mmol), palladium acetate (0.016 g, 0.071 mmol) and dicyclohexylphosphinobiphenyl (0.050 g, 0.14 mmol) were dissolved in THF (3 mL, degassed by bubbling with nitrogen for 30 min). The reaction mixture was placed under nitrogen, heated to 40° C. and stirred for 16 h. 5% NaOH (4 mL) and dichloromethane (10 mL) were added and the biphasic mixture was stirred about 15 min. Water was added and the mixture was extracted three times with dichloromethane. The combined organic extracts were dried over sodium sulfate, filtered and concentrated. Silica gel chromatography (0 to 10% methanol in dichloromethane:ethyl acetate (1:1)) afforded 0.415 g (81%) of the desired product as a yellow powder.

### Step D: 4-(3,4-Difluoro-phenyl)-7-fluoro-2-(4-methyl-piperazin-1-yl)-quinazoline

(0.360 g, 1.01 mmol) in dichloroethane (4 mL) was treated with proton sponge (0.13 g, 0.60 mmol), 1-chloroethyl chloroformate (0.220 mL, 2.01 mmol) and heated to reflux. After about 2 h, the reaction mixture was concentrated to about 2 mL and immediately purified by silica gel chromatography (10-70% ethyl acetate in hexanes). The resultant carbamate intermediate was dissolved in methanol (10 mL) and heated to reflux for 16 h. The reaction mixture was concentrated and dried in a vacuum oven at 40° C. to afford 0.290 g (76%) the title compound as a light yellow powder. MS (APCI) M+1=359.1; Elemental analysis found for C₁₈H₁₅F₃N₄.HCl: C, 56.89; H, 4.17; N, 13.96; Cl, 9.56; 1H NMR (400 MHz, CDCl3-D) d ppm 2.4 (s, 3 H) 2.6 (br. s., 4 H) 4.0 (br. s., 4 H) 6.9 (ddd, J=9.2, 8.0, 2.4 Hz, 1 H) 7.2 (m, 1 H) 7.3 (dt, J=10.0, 8.2 Hz, 1 H) 7.5 (m, 1 H) 7.6 (ddd, J=10.7, 7.7, 2.1 Hz, 1 H) 7.8 (dd, J=9.2, 6.2 Hz, 1 H).

### Example 113

### 4-(3-chloro-phenyl)-2-piperazin-1-yl-quinazoline

The titled compound was prepared in a manner similar to Example 18. MS (APCI) M+1=325.1; 1H NMR (400 MHz, DMSO-D6) d ppm 3.19 (m, 4H), 4.10 (m, 4H), 7.32 (m, 1H), 7.66 (m, 4H), 7.79 (m, 3H), 9.21 (bs, 2H).

### Example 114

### 4-(3,4-Difluoro-phenyl)-2-(4-methyl-piperazin-1-yl)-quinazoline

The titled compound was prepared in a manner similar to Example 140. MS (APCI) M+1=341.2; 1H NMR (400 MHz, DMSO-D6) d ppm 2.19 (s, 3 H), 2.37 (m, 4H), 3.83 (m, 4H), 7.21 (m, 1H), 7.33 (t, J=8.05 Hz, 4H), 7.58 (d, J=8.30 Hz, 1H), 7.70 (m, 2H).

### Example 115

### 4-(3,4-dichloro-phenyl)-2-piperazin-1-yl-quinazoline

MS (APCI) M+1=361.1; 1H NMR (400 MHz, DMSO-D6) d ppm 3.19 (m, 4H), 4.10 (m, 4H), 7.32 (m, 1H), 7.64 (d, J=8.05 Hz, 1H), 7.72 (dd, J=8.30, 1.95 Hz, 1H), 7.79 (m, 2H), 7.85 (d, J=8.05 Hz, 1H), 7.99 (d, J=1.95 Hz, 1H), 9.24 (bs, 2H).

### Example 116

### R-[4-(2,3-Difluoro-phenyl)-7-fluoro-quinazolin-2-yl]-pyrrolidin-3-yl-amine

The titled compound was prepared in a manner similar to Example 139. MS (APCI) M+1=345.1; Elemental analysis found for C₁₈H₁₅F₃N₄.2HCl: C, 51.70; H, 4.51; N, 12.65; Cl, 14.18; 1H NMR (400 MHz, CD30D-D4) d ppm 2.3 (m, 1 H) 2.5 (m, 1 H) 3.5 (m, 2 H) 3.6 (m, 1 H) 3.7 (dd, J=12.0, 6.6 Hz, 1 H) 4.9 (br. s., 1 H) 7.3 (br. s., 1 H) 7.4 (m, 2 H) 7.6 (m, 2 H) 7.8 (br. s., 1 H).

Examples 117-123 were prepared in a manner similar to Example 112.

### Example 117

### 7-Fluoro-4-(4-fluoro-2-methyl-phenyl)-2-piperazin-1-yl-quinazoline

MS (APCI) M+1=341.1; Elemental analysis found for C₁₉H₁₈F₂N₄.HCl: C, 59.67; H, 4.94; N, 14.31; Cl, 9.29; 1H NMR (400 MHz, CD30D-D4) d ppm 2.1 (s, 3 H) 3.3 (m, 4 H) 4.2 (m, 4 H) 7.1 (m, 2 H) 7.2 (dd, J=9.6, 2.6 Hz, 1 H) 7.3 (m, 2 H) 7.5 (dd, J=9.3, 6.1 Hz, 1 H).

### Example 118

### 7-Chloro-4-(4-fluoro-2-methyl-phenyl)-2-piperazin-1-yl-quinazoline

MS (APCI) M+1=357.1; Elemental analysis found for C₁₉H₁₈FClN₄.HCl: C, 56.35; H, 4.83; N, 13.49; Cl, 17.37; 1H NMR (400 MHz, CD30D-D4) d ppm 2.1 (s, 3 H) 3.3 (m, 4 H) 4.2 (m, 4 H) 7.1 (ddd, J=8.8, 2.6 Hz, 1 H) 7.3 (m, 2 H) 7.4 (m, 1 H) 7.4 (m, 1 H) 7.5 (dd, J=9.2, 6.2 Hz, 1 H).

### Example 119

### 4-(3,4-Difluoro-phenyl)-7-fluoro-2-piperazin-1-yl-quinazoline

MS (APCI) M+1=345.1; Elemental analysis found for C₁₈H₁₅F₃N₄.HCl: C, 56.89; H, 4.17; N, 13.96; Cl, 9.56; 1H NMR (400 MHz, CD30D-D4) d ppm 3.3 (m, 4 H) 4.2 (m, 4 H) 7.1 (ddd, J=9.2, 8.5, 2.6 Hz, 1 H) 7.3 (dd, J=10.5, 2.7 Hz, 1 H) 7.5 (dt, J=10.4, 8.2 Hz, 1 H) 7.6 (m, 1 H) 7.7 (ddd, J=11.1, 7.7, 2.2 Hz, 1 H) 7.9 (dd, J=9.3, 6.1 Hz, 1 H).

### Example 120

### 4-(2,4-Dichloro-phenyl)-7-fluoro-2-piperazin-1-yl-quinazoline

MS (APCI) M+1=377.0; Elemental analysis found for C₁₈H₁₅Cl₂FN₄.HCl.H₂O: C, 51.69; H, 4.50; N, 12.39; Cl, 22.31; 1H NMR (400 MHz, CD30D-D4) d ppm 3.3 (m, 4 H) 4.2 (m, 4 H) 7.1 (m, 1 H) 7.3 (dd, J=10.4, 2.6 Hz, 1 H) 7.5 (m, 2 H) 7.6 (m, 1 H) 7.7 (d, J=2.2 Hz, 1 H).

### Example 121

### 4-(2,3-Difluoro-phenyl)-6-fluoro-2-piperazin-1-yl-quinazoline

Elemental analysis found for C₁₈H₁₆F₂N₄.HCl: C, 56.68; H, 4.34; N, 14.18; Cl, 9.20; 1H NMR (400 MHz, METHANOL-D4) d ppm 3.3 (m, 4 H) 4.8 (s, 4 H) 7.2 (m, 1 H) 7.4 (m, 2 H) 7.5 (m, 1 H) 7.6 (m, 1 H) 7.8 (dd, J=9.3, 5.1 Hz, 1 H)

### Example 122

### 4-(2,4-Difluoro-phenyl)-6-fluoro-2-piperazin-1-yl-quinazoline

Elemental analysis found for C₁₈H₁₅F₃N₄.HCl: C, 56.94; H, 4.32; N, 14.21; Cl, 9.17; 1H NMR (400 MHz, METHANOL-D4) d ppm 3.3 (m, 4 H) 4.2 (m, 4 H) 7.2 (m, 3 H) 7.6 (m, 2 H) 7.7 (m, 1 H).

### Example 123

### 4-(2,3-Difluoro-phenyl)-6,7-difluoro-2-piperazin-1-yl-quinazoline

Elemental analysis found for C₁₈H₁₄F₄N₄.HCl: C, 52.45; H, 3.72; N, 13.49; Cl, 10.58; 1H NMR (400 MHz, METHANOL-D4) d ppm 3.4 (m, 4 H) 4.3 (m, 4 H) 7.4 (m, 2 H) 7.5 (m, 2 H) 7.7 (dd, J=11.3, 7.2 Hz, 1 H).

### Example 124

### S-4-(2,3-Difluoro-phenyl)-7-fluoro-2-(2-methyl-piperazin-1-yl)-quinazoline

The titled compound was prepared in a manner similar to Example 139. MS (APCI) M+1=359.1; Elemental analysis found for C₁₉H₁₇F₃N₄.2HCl: C, 54.04; H, 4.57; N, 12.51; Cl, 12.84; 1H NMR (400 MHz, CD30D-D4) d ppm 1.5 (d, J=7.1 Hz, 3 H) 3.2 (m, 1 H) 3.4 (m, 2 H) 3.5 (m, 1 H) 3.6 (m, 1 H) 5.0 (dd, J=15.0, 3.3 Hz, 1 H) 5.4 (m, 1 H) 7.2 (td, J=8.8, 2.4 Hz, 1 H) 7.4 (m, 2 H) 7.6 (m, 2 H) 7.8 (ddd, J=9.1, 6.0, 2.9 Hz, 1 H).

Examples 125 and 126 were prepared in a manner similar to Example 134.

### Example 125

### 4-(2,5-dichloro-phenyl)-2-piperazin-1-yl-quinazoline

MS (APCI) M−1=358.1; 1H NMR (400 MHz, DMSO-D6) d ppm 3.2 (m, 4 H) 4.1 (t, J=5.1 Hz, 4 H) 7.3 (dd, J=6.6, 1.2 Hz, 1 H) 7.3 (m, 1 H) 7.6 (m, 1 H) 7.7 (m, 3 H) 7.8 (m, 1 H) 9.1 (s, 2 H).

### Example 126

### 4-(3,5-difluoro-phenyl)-2-piperazin-1-yl-quinazoline

MS (APCI) M−1=327; 1H NMR (400 MHz, DMSO-D6) d ppm 3.2 (m, 4 H) 4.1 (m, 4 H) 7.3 (t, J=7.6 Hz, 1 H) 7.5 (m, 3 H) 7.6 (d, J=8.1 Hz, 1 H) 7.8 (m, 2 H) 9.2 (s, 2 H).

### Example 127

### S-4-(2,6-Difluoro-phenyl)-2-(2-methyl-piperazin-1-yl)-quinazoline

The titled compound was prepared in a manner similar to Example 139. MS (APCI) M+1=341.1; 1H NMR (400 MHz, DMSO-D6) d ppm 1.29 (d, J=7.08 Hz, 3H), 3.06 (m, 1H), 3.31 (m, 4H), 4.77 (d, J=14.64 Hz, 1H), 5.16 (m, 1H), 7.35 (m, 4H), 7.70 (m, 2H), 7.81 (m, 1H), 8.67 (bs, 1H), 9.12 (bs, 1H).

### Example 128

### 6-Chloro-4-phenyl-2-piperazin-1-yl-quinazoline

The titled compound was prepared in a manner similar to Example 18. MS (APCI) M−1=324.1; 1H NMR (400 MHz, DMSO-D6) d ppm 3.19 (m, 4H), 4.12 (m, 4H), 7.62 (m, 4H), 7.75 (m, 4H), 9.34 (s, 2H).

### Example 129

### 4-(2-Fluoro-phenyl)-6-chloro-2-piperazin-1-yl-quinazoline

The titled compound was prepared in a manner similar to Example 112. Elemental analysis found for C₁₈H₁₆ClFN₄.HCl: C, 56.30; H, 4.68; N, 13.84; Cl, 8.87; 1H NMR (400 MHz, METHANOL-D4) d ppm 3.3 (m, 4 H) 4.2 (s, 4 H) 7.5 (m, 3 H) 7.7 (m, J=9.8 Hz, 4 H).

### Example 130

### R-4-(2,3-Difluoro-phenyl)-7-fluoro-2-(2-methyl-piperazin-1-yl)-quinazoline

The titled compound was prepared in a manner similar to Example 139. MS (APCI) M+1=359.1; Elemental analysis found for C₁₉H₁₇F₃N₄.2HCl: C, 54.04; H, 4.57; N, 12.51; Cl, 12.84; 1H NMR (400 MHz, CD30D-D4) d ppm 1.5 (d, J=7.1 Hz, 3 H) 3.2 (m, 1 H) 3.4 (m, 2 H) 3.5 (m, 1 H) 3.6 (m, 1 H) 5.0 (dd, J=15.0, 3.3 Hz, 1 H) 5.4 (m, 1 H) 7.2 (td, J=8.8, 2.4 Hz, 1 H) 7.4 (m, 2 H) 7.6 (m, 2 H) 7.8 (ddd, J=9.1, 6.0, 2.9 Hz, 1 H).

### Example 131

### 4-(2,6-Difluoro-phenyl)-6-chloro-2-piperazin-1-yl-quinazoline

The titled compound was prepared in a manner similar to Example 112. Elemental analysis found for C₁₈H₁₅ClF₂N₄.HCl: C, 52.10; H, 4.17; N, 13.40; Cl, 8.78; 1H NMR (400 MHz, METHANOL-D4) d ppm 3.3 (m, 4 H) 4.2 (m, 4 H) 7.2 (m, 2 H) 7.4 (d, J=1.5 Hz, 1 H) 7.7 (m, 2 H) 7.8 (m, 1 H).

### Example 132

### N1-[4-(2,4-Difluoro-phenyl)-quinazolin-2-yl]-ethane-1,2-diamine

Examples 132, 148, 149, 150, 151, 152, 153, 154, 155, 156, 157, and 158 were prepared as follows: template (an appropriately substituted 2-Chloro-4-phenyl-quinazoline) (0.1807 mmol), 0.317 ml of an appropriately substituted amine (e.g., 1-methyl-pyrrolidin-3-ylamine, methyl-piperidin-4-yl-amine, 4-amino-piperidine-1-carboxylic acid tert-butyl ester, 3-aminomethyl-pyrrolidine-1-carboxylic acid tert-butyl ester, etc.) (0.6325 mmol), 3 ml of toluene, and 3 drops of pyridine are combined in a vial. The reaction is stirred and refluxed at 111° C. overnight. The solvent is removed in vacuo and purified using Waters Fractionlynx LC/MS apparatus with a Xterra RP-18 5 micron, 30×100 mm column (supplied from Waters) and running 10% acetonitrile (with 3% 1-propanol): 90% water (with 3% 1-propanol) as solvent for the first 7 minutes and then switching to 100% acetonitrile (with 3% 1-propanol) for the remaining 3 minutes of the run. Samples containing BOC groups were further subjected to 3 ml of 25% TFA (trifluoroacetic acid) in dichloromethane and shaken at room temperature for 4 hours. The solvent is removed in vacuo and the samples are purified using a Xterra RP-18 5 micron, 30×100 mm column, and running 15% acetonitrile (with 3% 1-propanol): 85% water (with 3% 1-propanol) as the solvent for the first 7 minutes, then switching to 100% acetonitrile (with 3% 1-propanol) for the remaining 3 minutes of the run. The average yield was 36.17 mg (0.1063 mmol, 58.85% yield) of desired product, with an average purity of 99.42% after purification. For the titled compound: MS (APCI) M+1=301.2; 1H NMR (400 MHz, CDCl3) d ppm 1.2 (s, 2 H) 2.9 (m, 2 H) 3.6 (m, 2 H) 5.8 (s, 1H) 6.9 (m, 2 H) 7.1 (m, 1 H) 7.4 (m, 2 H) 7.6 (m, 2 H).

### Example 133

### 8-Fluoro-4-phenyl-2-piperazin-1-yl-quinazoline

8-Fluoro4-phenyl-2-piperazin-1-yl-quinazoline was prepared as in Example 139, except that a piperazine was used instead of 1-methyl-piperazine. Piperazine (290 mg, 3.4 mmol) was added to a solution of 2-chloro-8-fluoro-4-phenyl-quinazoline (500 mg, 1.93 mmol) in dichloromethane (10 mL). Reaction mixture stirred at room temperature for 16 hours. The mixture was then diluted with dichloromethane (50 mL) and washed with 5% aqueous NaOH (15 mL). Organic layer was dried over Na₂SO₄, filtered and concentrated in vacuo. Silica gel chromatography of the residue (0-5% methanol in dichloromethane) afforded a yellow oil. Treatment of the oil with 2M HCl in diethyl ether gave a yellow solid that was collected by filtration and dried in a 45° C. vacuum oven to yield 0.202 mg (43%) of 8-Fluoro-4-phenyl-2-piperazin-1-yl-quinazoline. MS (APCI) M+1=309.2; 1H NMR (400 MHz, CHLOROFORM-D) d ppm 3.0 (m, 4 H) 4.0 (m, 4 H) 7.0 (td, J=8.1, 4.9 Hz, 1 H) 7.3 (ddd, J=10.7, 7.6, 1.2 Hz, 1 H) 7.5 (m, 3 H) 7.6 (d, J=8.5 Hz, 1 H) 7.7 (m, 1 H) 7.7 (d, J=2.2 Hz, 1 H).

### Example 134

### 4-(2-Chloro-4-fluoro-phenyl)-7-fluoro-2-piperazin-1-yl-quinazoline

4-(2-Chloro-4-fluoro-phenyl)-7-fluoro-2-piperazin-1-yl-quinazoline. Example 134 was prepared as in Example 112 except that the reaction involving the boronic acid was replaced with the following step. 2-chloro-4-fluorophenylzinc iodide in 0.5M tetrahydrofuran and a catalytic amount of 1,1-bis(diphenylphosphino)ferrocenepalladium(II) chloride was added to a suspension of 4-Chloro-7-fluoro-2-(4-methyl-piperazin-1-yl)-quinazoline in toluene. The reaction was heated to reflux for 6 hours. The solvent was removed under reduced pressure, diluted with ethyl acetate and washed with water. The filtrate was dried with magnesium sulfate, filtered, concentrated and purified by chromatography on silica gel using 5% methanol/dichloromethane as eluent. The solvent was removed under reduced pressure to afford 4-(2-Chloro-4-fluoro-phenyl)-7-fluoro-2-(4-methyl-piperazin-1-yl)-quinazoline as a solid. MS (APCI) M−1=361.1; 1H NMR (400 MHz, DMSO-D6) d ppm 3.2 (m, 4 H) 4.1 (m, 4 H) 7.2 (m, 1 H) 7.4 (dd, J=10.6, 2.6 Hz, 1 H) 7.7 (dd, J=9.2, 6.2 Hz, 1 H) 7.8 (m, 1 H) 7.9 (m, 1 H) 8.1 (d, J=7.8 Hz, 1 H) 9.3 (s, 2H).

Examples 135-137 were prepared in a manner similar to Example 134.

### Example 135

### 7-Fluoro-2-piperazin-1-yl-4-thiazol-2-yl-quinazoline

MS (APCI) M−1=316.0; 1H NMR (400 MHz, DMSO-D6) d ppm 3.2 (m, 4 H) 4.1 (m, 4 H) 7.3 (m, 2 H) 8.1 (d, J=3.2 Hz, 1 H) 8.2 (d, J=3.2 Hz, 1 H) 9.3 (s H) 9.5 (m, 1 H).

### Example 136

### 4-(2-Methoxy-phenyl)-7-fluoro-2-piperazin-1-yl-quinazoline

MS (APCI) M−1=339.1; 1H NMR (400 MHz, DMSO-D6) d ppm 3.2 (m, 4 H) 3.7 (s, 3 H) 4.1 (m, 4 H) 7.1 (m, 2 H) 7.3 (d, J=2.4 Hz, 1 H) 7.3 (m, 1 H) 7.4 (dd, J=9.2, 6.5 Hz, 1 H) 7.5 (m, 1 H) 9.4 (s, 2 H).

### Example 137

### 7-Fluoro-4-(5-fluoro-2-methyl-phenyl)-2-piperazin-1-yl-quinazoline

MS (APCI) M−1=341.1; 1H NMR (400 MHz, DMSO-D6) d ppm 2.0 (s, 3 H) 3.2 (m, 4 H) 4.1 (m, 4 H) 7.1 (m, 1 H) 7.2 (dd, J=9.2, 2.8 Hz, 1 H) 7.3 (m, 1 H) 7.3 (m, J=8.3 Hz, 1 H) 7.4 (m, 2 H) 9.5 (s, 2 H).

### Example 138

### R-4-(2,4-Difluoro-phenyl)-7-fluoro-2-(2-methyl-piperazin-1-yl)-quinazoline

The titled compound was prepared in a manner similar to Example 139. MS (APCI) M−1=345.1; 1H NMR (400 MHz, DMSO-D6) d ppm 3.2 (m, 4 H) 4.1 (m, 4 H) 7.2 (m, 1 H) 7.4 (d, J=12.9 Hz, 1 H) 7.5 (m, 3 H) 7.9 (dd, J=9.3, 6.3 Hz, 1 H) 9.1 (s, 2 H).

### Example 139

### S-4-(2,4-Difluoro-phenyl)-7-fluoro-2-(2-methyl-piperazin-1-yl)-quinazoline

S4-(2,4-Difluoro-phenyl)-7-fluoro-2-(2-methyl-piperazin-1-yl)-quinazoline was prepared as in Example 140, except that a 4N-BOC-2-methylpiperazine was used instead of 1-methyl-piperazine, and the benzyloxycarbonyl (Cbz) group was removed using palladium on carbon. (S)-4-N-BOC-2-methylpiperazine (0.507 mg, 2.55 mmol) was added to a suspension of 2-Chloro-4-(2,4-difluoro-phenyl)-7-fluoro-quinazoline (300 mg, 1.02 mmol) in toluene (7 mL). Reaction mixture stirred at reflux 20 hours. HPLC analysis indicated that the reaction was not complete. A catalytic amount of piperazine was added and reaction mixture continued to heat for an additional 24 hours. Solvent was removed under reduced pressure to afford 4-[4-(2,4-Difluoro-phenyl)-7-fluoro-quinazolin-2-yl]-3-methyl-piperazine-1-carboxylic acid tert-butyl ester as a solid. Hydrochloric acid (2M in ethyl ether) (3.0 mL) was added to a suspension of) 4-[4-(2,4-Difluoro-phenyl)-7-fluoro-quinazolin-2-yl]-3(s)-methyl-piperazine-1-carboxylic acid tert-butyl ester (0.382 g, 0.833 mL) in dichloromethane (10 mL). Reaction stirred at room temperature for 24 hours. Solvent was removed under reduced pressure to afford 4-(2,4-Difluoro-phenyl)-7-fluoro-2-(2-methyl-piperazin-1-yl)-quinazoline as a solid product. MS (APCI) M−1=359.1; 1H NMR (400 MHz, DMSO-D6) d ppm 1.3 (d, J=6.8 Hz, 3 H) 2.7 (m, 1 H) 3.0 (m, 2 H) 3.1 (m, 2 H) 4.6 (m, J=12.2 Hz, 1 H) 5.0 (m, 1 H) 7.1 (m, 1 H) 7.3 (m, 2 H) 7.5 (m, 2 H) 7.7 (m, 1 H).

### Example 140

### 4-(2,4-Difluoro-phenyl)-7-fluoro-2-piperazin-1-yl-quinazoline

4-(2,4-Difluoro-phenyl)-7-fluoro-2-piperazin-1-yl-quinazoline was prepared in a manner similar to Example 18, except N-chlorosuccinimide and triphenyl phosphine were used instead of phosphorous pentachloride and phosphorous oxychloride as follows: triphenyl phosphine (3000 mg, 115 mmol) was slowly added to a suspension of N-chlorosuccinimide (1500 mg 115 mmol) in dioxane (400 mL). The reaction mixture stirred at room temperature for a half an hour then added 4-(2,4-Difluoro-phenyl)-7-fluoro-1H-quinazolin-2-one (4.8 g, 17.4 mmol) and heated to reflux for 24 hours. The reaction mixture was cooled to room temperature, quenched with triethylamine (150 mL), concentrated under reduced pressure, dissolved in ethyl acetate (200 mL) and stirred for one hour. Solvent was removed under reduced pressure and purified by chromatography on silica gel using 5% ethyl acetate/hexanes as eluent. Solvent was removed under reduced pressure to afford 2-Chloro-4-(2,4-difluoro-phenyl)-7-fluoro-quinazoline as a white solid. MS (APCI) M−1=345.0; 1H NMR (400 MHz, DMSO-D6) d ppm 2.8 (m, 4 H) 3.8 (m, 4 H) 7.1 (m, 1 H) 7.3 (m, 2 H) 7.5 (m, 2 H) 7.7 (m, 1H).

Examples 141-144 were prepared in a manner similar to Example 58.

### Example 141

### Azetidin-3-yl-[4-(2,4-difluoro-phenyl)-7-fluoro-quinazolin-2-yl]-amine

MS (APCI) M−1=331.0; 1H NMR (400 MHz, DMSO-D6) d ppm 4.1 (m, 2 H) 4.2 (m, 2 H) 4.9 (m, 1 H) 7.1 (m, 1 H) 7.3 (m, 1 H) 7.4 (m, 1 H) 7.5 (m, 2 H) 7.7 (m, 1 H) 8.5 (s, 1 H) 9.1 (s, 2 H).

### Example 142

### R,R-4-(2,4-Difluoro-phenyl)-7-fluoro-2-(hexahydro-pyrrolo[3,4-b]pyrrol-1-yl)-quinazoline

MS (APCI) M−1=371.1; 1H NMR (400 MHz, DMSO-D6) d ppm 2.0 (m, 1 H) 2.1 (m, 1 H) 3.1 (m, 2 H) 3.3 (m, 3 H) 3.7 (m, 1 H) 3.8 (m, 1 H) 4.6 (dd, J=13.2, 2.9 Hz, 1 H) 7.1 (t, J=8.9 Hz, 1 H) 7.3 (m, 2 H) 7.5 (m, 2 H) 7.7 (m, 1 H).

### Example 143

### R-[4-(2,4-Difluoro-phenyl)-7-fluoro-quinazolin-2-yl]-pyrrolidin-3-yl-amine

MS (APCI) M−1=345.1; 1H NMR (400 MHz, DMSO-D6) d ppm 2.1 (m, 1 H) 2.3 (m, 1 H) 3.7 (m, 1 H) 3.8 (m, 4 H) 7.1 (m, 1 H) 7.3 (m, 1 H) 7.5 (m, 3 H) 7.7 (m, 1 H) 8.4 (s, 2 H).

### Example 144

### S-[4-(2,4-Difluoro-phenyl)-7-fluoro-quinazolin-2-yl]-pyrrolidin-3-yl-amine

MS (APCI) M−1=345.1; 1H NMR (400 MHz, METHANOL-D4) d ppm 2.4 (m, 1 H) 2.7 (m, 1 H) 4.1 (m, 1 H) 4.2 (m, 4 H) 7.3 (m, 2 H) 7.4 (m, 1 H) 7.8 (m, 1 H) 7.8 (m, 1 H) 7.9 (m, 1 H).

Examples 145-147 were prepared in a manner similar to Example 112.

### Example 145

### 5-Methyl-4-phenyl-2-piperazin-1-yl-quinazoline

MS (APCI) M−1=305.1; 1H NMR (400 MHz, DMSO-D6) d ppm 1.9 (s, 3 H), 3.2 (s, 4 H), 4.1 (m, 4 H), 7.1 (d, J=7.1 Hz, 1 H), 7.5 (m, 6 H), 7.6 (m, 1 ), 9.2 (s, 2 H).

### Example 146

### 4-(2,6-Difluoro-phenyl)-6,7-difluoro-2-piperazin-1-yl-quinazoline

Elemental analysis found for C₁₈H14F₄N₄.HCl: C, 52.54; H, 3.60; N, 13.29; Cl, 8.2; 1H NMR (400 MHz, METHANOL-D4) d ppm 3.3 (m, 4 H) 4.2 (m, 4 H) 7.2 (m, 3 H) 7.5 (m, 1 H) 7.7 (m, J=6.6 Hz, 1 H).

### Example 147

### 4-(2,4-Difluoro-phenyl)-6,7-difluoro-2-piperazin-1-yl-quinazoline

Elemental analysis found for C₁₈H₁₄F₄.HCl: C, 52.54; H, 3.60; N, 13.29; Cl, 8.2; 1H NMR (400 MHz, METHANOL-D4) d ppm 3.3 (m, 4 H) 4.2 (m, 4 H) 7.2 (m, 2 H) 7.4 (m, 1 H) 7.5 (m, 1 H) 7.6 (m, 1 H).

Examples 148-158 were prepared as in Example 132.

### Example 148

### [4-(2,6-Difluoro-phenyl)-quinazolin-2-yl]-piperidin-4-yl-amine

MS (APCI) M+1=341.2.

### Example 149

### {1-[4-(2,6-Difluoro-phenyl)-quinazolin-2-yl)-pyrrolidin-3-yl}-methyl-amine

MS (APCI) M+1=341.2.

### Example 150

### {1-[4-(2,4-Difluoro-phenyl)-quinazolin-2-yl]-pyrrolidin-3-yl}-methyl-amine

MS (APCI) M+1=341.2.

### Example 151

### {1-[4-(2,6-Difluoro-phenyl)-quinazolin-2-yl]-piperidin-4-yl}-methyl-amine

MS (APCI) M+1=355.2.

### Example 152

### N1-[4-(2,4-Difluoro-phenyl)-quinazolin-2-yl]-propane-1,3-diamine

MS (APCI) M+1=315.2.

### Example 153

### {1-[4-(2,4-Difluoro-phenyl)-quinazolin-2-yl]-piperidin-4-yl}-methyl-amine

MS (APCI) M+1=355.2.

### Example 154

### [4-(2,4-Difluoro-phenyl)-quinazolin-2-yl]-piperidin-4-yl-amine

MS (APCI) M+1=341.2.

### Example 155

### [4-(2,4-Difluoro-phenyl)-quinazolin-2-yl]-pyrrolidin-3-ylmethyl-amine

MS (APCI) M+1=341.2.

### Example 156

### [4-(2,3-Difluoro-phenyl)-quinazolin-2-yl]-piperidin-4-yl-amine

MS (APCI) M+1=341.2.

### Example 157

### {1-[4-(2,3-Difluoro-phenyl)-quinazolin-2-yl]-pyrrolidin-3-yl}-methyl-amine

MS (APCI) M+1=341.2.

### Example 158

### {1-[4-(2,3-Difluoro-phenyl)-quinazolin-2-yl-piperidin-4-yl}-methyl-amine

MS (APCI) M+1=355.2.

### Example 159

### N1-[4-(2,3-Difluoro-phenyl)-quinazolin-2-yl]-propane-1,3-diamine

The titled compound was prepared in accordance with Example 161. MS (APCI) M+1=315.2.

### Example 160

### 7-Fluoro-2-piperazin-1-yl-4-(2-trifluoromethyl-phenyl)-quinazoline

The titled compound was prepared in a manner similar to Example 112. MS (APCI) M+1=377.1; Elemental analysis found for C₁₉H₁₆F₄N₄.HCl: C, 53.44; H, 4.22; N, 12.77; Cl, 9.78; 1H NMR (400 MHz, CD30D-D4) d ppm 3.3 (m, 4 H) 4.2 (dd, J=6.1, 4.6 Hz, 4 H) 7.1 (m, 1 H) 7.3 (dd, J=10.2, 2.4 Hz, 1 H) 7.4 (dd, J=9.0, 6.1 Hz, 1 H) 7.5 (m, 1 H) 7.8 (m, 2 H) 7.9 (m, 1 H).

### Example 161

### 2-(2,4-Difluoro-phenyl)-4-piperazin-1-yl-quinazoline

2-(2,4-Difluoro-phenyl)-4-piperazin-1-yl-quinazoline was prepared as in Example 112 except that instead of reacting the quinazoline-2,4-dione with dimethylpiperazine, tripropylamine, and phosphorous oxychloride, the following were carried out. Quinazoline-2,4-dione (10.0 g, 61.7 mmol) was dissolved in phosphorous oxychloride (56 mL, 617 mmol) and treated slowly with dimethylaniline (15.6 mL, 123 mmol). The reaction mixture was heated to 100° C., stirred for 16 h, cooled and concentrated. The residue was dissolved in dichloromethane, cooled to 0° C., and carefully treated with water to quench the remaining phosphorous oxychloride. The organic layer was separated, washed twice with water, dried over sodium sulfate (anhydrous), filtered and concentrated. The residue was recrystallized from hot isopropanol:water (10:1) to afford 4.0 g (33%) of 2,4-Dichloro-quinazoline.

2,4-Dichloro-quinazoline (1.0 g, 5.0 mmol) in THF (10 mL) was treated dropwise with methylpiperazine (0.56 mL, 5.0 mmol) and stirred at for 2 h. An additional 0.28 mL (2.5 mmol) methylpiperazine was added and the mixture was stirred another 1.5 h. Dichloromethane and 5% NaOH were added, and the organic layer was separated. The aqueous layer was extracted twice with dichloromethane and the combined organic layers were dried over sodium sulfate, filtered and concentrated. Flash chromatography on silica gel (0-10% methanol in dichloromethane) afforded 1.27 g. (96%) of 2-Chloro-4-(4-methyl-piperazin-1-yl)-quinazoline. MS (APCI) M+1=327.1; Elemental analysis found for C₁₈H₁₆F₂N₄.HCl: C, 57.13; H, 5.10; N, 13.99; Cl, 9.87; 1H NMR (400 MHz, CD30D-D4) d ppm 3.5 (m, 4 H) 4.1 (m, 4 H) 7.1 (m, 2 H) 7.7 (ddd, J=8.4, 6.9, 1.3 Hz, 1 H) 7.9 (ddd, J=8.4, 7.0, 1.5 Hz, 1 H) 8.0 (ddd, J=8.5, 1.3, 0.6 Hz, 1 H) 8.1 (m, 2 H).

### Example 162

### 4-(2,6-Difluoro-phenyl)-7-fluoro-2-piperidin-4-yl-quinazoline

The titled compound was prepared in a manner similar to Example 41. MS (APCI) M+1=344.1; 1H NMR (400 MHz, DMSO-D6) d ppm 2.1 (m, 2 H) 2.2 (m, 2 H) 3.1 (m, 2 H) 3.3 (d, J=3.9Hz, 1 H) 3.4 (m, 2 H) 7.4 (m, 2 H) 7.6 (m, 1 H) 7.8 (m, 2 H) 7.9 (d, J=2.4 Hz, 1 H) 8.7 (s, 1 H) 9.1 (s, 1 H).

### Example 163

### 7,8-Difluoro-4-phenyl-2-piperazin-1-yl-quinazoline

The titled compound was prepared in a manner similar to Example 139. MS (APCI) M+1=327.1; 1H NMR (400 MHz, METHANOL-D4) d ppm 3.3 (m, 4 H) 4.3 (m, 4 H) 7.2 (ddd, J=10.2, 9.3, 7.1 Hz, 1 H) 7.6 (m, 3 H) 7.7 (m, 3 H).

Examples 164-167 were prepared as in Example 140, except that 1-benzyl-3-isopropylpiperazine or 1-benzyl-3-ethylpiperazine was used instead of 1-methyl-piperazine.

### Example 164

### S-4-(2,6-Difluoro-phenyl)-2-(2-ethyl-piperazin-1-yl)-quinazoline

Elemental analysis found for C₂₀H₂₀F₂N₄.HCl: C, 56.48; H, 5.40; N, 12.82; Cl, 13.04; 1H NMR (400 MHz, METHANOL-D4) d ppm 1.0 (t, J=7.3 Hz, 3 H) 1.9 (m, 1 H) 2.0 (m, 1 H) 3.3 (m, 1 H) 3.4 (dd, J=13.2, 4.1 Hz, 1 H) 3.6 (m, 3 H) 5.0 (d, J=14.4 Hz, 1 H) 5.3 (m, 3 H) 7.2 (t, J=8.7 Hz, 2 H) 7.4 (m, 1 H) 7.6 (d, J=7.8 Hz, 1 H) 7.7 (m, 1 H) 7.9 (m, 2 H).

### Example 165

### S-4-(2,4-Difluoro-phenyl)-2-(2-ethyl-piperazin-1-yl)-quinazoline

MS (APCI) M+1=355.1; 1H NMR (400 MHz, DMSO-D6) d ppm 0.8 (t, 3 H) 1.8 (m, 1 H) 1.9 (m, 1 H) 2.9 (m 1 H) 3.1 (m, 1 H) 3.3 (m, 3 H) 4.9 (m, 1 H) 5.1 (m, 1 H) 7.2 (m, 1 H) 7.3 (m, 1 H) 7.5 (m, 2 H) 7.6 (m, 1 H) 7.7 (m, 1 H) 7.8 (ddd, J=8.5, 6.9, 1.5 Hz, 1 H).

### Example 166

### S-4-(2,3-Difluoro-phenyl)-2-(2-isopropyl-piperazin-1-yl)-quinazoline

MS (APCI) M+1=369.2; 1H NMR (400 MHz, DMSO-D6) d ppm 0.8 (d, J=6.6 Hz, 4 H) 1.0 (d, J=6.3 Hz, 3 H) 3.0 (m, 1 H) 3.1 (m, 1 H) 3.3 (m, 3 H) 3.5 (m, 1 H) 4.8 (m, 1 H) 5.0 (m, 1 H) 7.3 (m, 1 H) 7.4 (m, 2 H) 7.5 (m, 1 H) 7.6 (d, J=8.5 Hz, 1 H) 7.7 (m, 1 H) 7.8 (ddd, J=8.5, 7.0, 1

### Example 167

### S-4-(2,4-Difluoro-phenyl)-2-(2-isopropyl-piperazin-1-yl)-quinazoline

A solution of 2-chloro-4-(2,4-difluoro-phenyl)-quinazoline (300 mg, 1.086 mmol) and 1-benzyl-3-isopropylpiperazine (585 mg, 2.68 mmol) in toluene (5 mL) was heated to 145° C. for 24 h. The solvent evaporated to provide a brown oil. The reaction was cooled and purified by silica gel chromatography (15% diethyl ether/hexanes) to provide 369 mg (74%) of 2-(4-Benzyl-2-isopropyl-piperazin-1-yl)-4-(2,4-difluoro-phenyl)-quinazoline as an orange oil. MS (APCI) M+1=369.2; 1H NMR (400 MHz, DMSO-D6) d ppm 0.8 (d, J=6.6 Hz, 3 H) 1.0 (d, J=6.3 Hz, 3 H) 2.5 (m, 1 H) 3.0 (m, 1 H) 3.1 (m, 1 H) 3.3 (m, 2 H) 3.5 (d, J=12.9 Hz, 1 H) 4.8 (dd, J=11.3, 3.5 Hz, 1 H) 5.0 (m, 1 H) 7.25 (ddd, J=8.2, 6.9, 1.2 Hz, 1 H) 7.3 (td, J=8.2, 2.4 H.

### 2-Bromophenyl Carbamic Acid Methyl Ester

To a solution of 2-bromoaniline (50.0 g, 291 mmol) and pyridine (58.8 mL, 726.6 mmol, 2.5 equiv) in CH₂Cl₂(300 mL), cooled in ice water bath under N₂, was added dropwise a solution of methyl chloroformate (27.0 mL, 348.8 mmol, 1.2 equiv) in CH₂Cl₂ (50 mL). A white solid formed. After the addition, the mixture was warmed to ambient temperature and stirred overnight. Water was added to dissolve the entire solid. The mixture was washed with 1N HCl (3×), saturated brine, saturated NaHCO₃, and dried over MgSO₄. Co-evaporation with heptane gave an oil, which was dried at 45° C. vacuum oven over night, to give 2-bromophenyl carbamic acid methyl ester as a light yellow oil, 65.11 g (97.4%).

### 4-(2-FIuorophenyl)-2(1H)-Quinazolinone (or 4-(2-Fluorophenyl)-2-hydroxy-quinazoline)

To a solution of 2-bromophenyl carbamic acid methyl ester (63.4 g, 276 mmol) in Et₂O (552 mL, 0.5 M), cooled in dry ice-MeCN bath (−40° C.), under N₂, was added dropwise a solution of n-BuLi (362 mL, 579 mmol, 2.1 equiv, 1.6 M, Aldrich). A bright yellow solid formed. The mixture was stirred at −20° C. for 30 min, and then a solution of 2-fluorobanzonitrile (30.8 mL, 289.4 mmol, 1.05 equiv) in ether (30 mL) was added slowly. The mixture turned to brick red-brown in color. The mixture was warmed to ambient temperature and stirred overnight (18 h). H₂O was added to quench the reaction, and the mixture was acidified with 1N HCl to pH˜2. The solid formed was filtered, washed with tBuOMe (2×), dried at 50° C. vacuum oven for 3 days to give a light yellow solid 58.47 g, mp>250° C. The filtrate was extracted with CH₂Cl₂ (3×), washed with saturated NaCl, NaHCO₃, and dried (MgSO₄). Removal of solvent gave a light yellow solid, which was dried at 50° C. vacuum oven for 18 h, to give 5.55 g of a 2^{nd} crop of 4-(2-Fluorophenyl)-2(1H)-Quinazolinone (or 4-(2-Fluorophenyl)-2-hydroxy-quinazoline) as a very light yellow solid, mp>250° C.

Total yield of 4-(2-Fluorophenyl)-2(1H)-Quinazolinone was 64.0 g, 96.7%.

### 4-(2-Fluorophenyl)-2-Chloro-quinazoline

A mixture of SOCl₂ (678.6 mL, 9.303 mole, 15 equiv), 4-(2-Fluorophenyl)-2(1H)-Quinazolinone (149.0 g, 602.2 mmol) and DMF (1.5 mL) was heated to reflux for 4 h, and then cooled to ambient temperature over night. All solid dissolved in 1 hr. The light brown mixture was poured very carefully and slowly to ice with stirring. Additional ice was added to cool the mixture. Solid formed was filtered, washed with water (3×), and left in the funnel with suction for 3 hrs to remove extra amount of water. The solid was dried in 50° C. vacuum oven for 20 h, to give 4-(2-Fluorophenyl)-2-Chloro-quinazoline as an off-white solid 154.02 g (96.0%), HPLC: 95%, mp 141° C.

### 4-[(2-Fluorophenyl)-2-Quinazolinyl)]-1-Piperazinecarboxylic acid 1,1 -dimethylethyl ester

To a slurry of 4-(2-fluorophenyl)-2-chloro-quinazoline (19.5 g, 75.4 mmol) and N-ethylmorpholine (24.0 mL, 188.5 mmol, 2.5 equiv) in i-PrOH (332 mL, 0.3 M) was added N-Boc piperazine (17.0 g, 90 mmol, 1.2 equiv). The light yellow slurry was heated to reflux for 3 h. All solid dissolved to give a light brown solution. Then solid formed again after 1.5 h. The mixture was cooled in ice water bath. The solid was filtered, washed with IPA, dried at 50° C. vacuum oven over night, to give almost white solid, 31.9 g (98.0 %), mp 183.5-185° C., HPLC purity: 94.6%.

### 4-[(2-Fluorophenyl)-2-(1 -Piperazinyl)-Quinazoline)

To the solid of 4-[(2-Fluorophenyl)-2-Quinazolinyl)]-1-Piperazinecarboxylic acid 1,1-dimethyl-ethyl ester (31.9 g, 78.1 mmol) was added a solution of trifluoroacetic acid (60.2 mL, 781 mmol, 10 equiv) in CH₂Cl₂ (150 mL, 40%), cooled in ice-water bath. The reaction was exothermic and gas bubble formed. The solution was stirred at ambient temperature for 2 h (MS showed there was no starting material, and a major product peak (M⁺+1:309)). Solvent was co-evaporated with heptane to give a brown foaming solid.

The foaming solid was dissolved in CH₂Cl₂ (100 mL) and a solution of Na₂CO₃ (83.0 g, 781.0 mmol, 10 equiv) dissolved in H₂O (500 mL) was added. The mixture was stirred at ambient temperature for 18 h. The aqueous layer was separated from the organic, and extracted with CH₂Cl₂ (2×). The combined organic was washed with saturated NaHCO₃ and H₂O, dried over MgSO₄. Evaporation of solvent, and co-evaporation with heptane gave a light yellow solid. The solid was dried at 50° C. vacuum oven for 20 h, to give a yellow solid, 19.26 g (80.0%). mp 133° C., HPLC purity: 99.3%, ¹H, ¹⁹F NMR.

### 4-[(2-Fluorophenyl)-2-(1 -Piperazinyl)-Quinazoline

A three-necked round bottom flask (RBF) was equipped with a mechanical stirrer, and a dropping funnel with glass wool at the bottom. At the top of the dropping funnel was fitted with a condenser. The dropping funnel was charged with solid chloride (10.0 g, 38.7 mmol). In the RBF, perizine (33.3 g, 387 mmol, 10 equiv) and N-methylmorpholine (6.38 mL, 58.0 mmol, 1.5 equiv) were dissolved in i-PrOH (500 mL) and heated to reflux. The i-PrOH was condensed into the dropping funnel and added to the RBF. It took 6 h to complete the addition. The mixture was refluxed for additional 2.5 h, and then stirred at ambient temperature over night (18 h). The mixture was concentrated to almost dry. H₂O (100 mL) was added, and stirred for 30 min. The solid was filtered, washed with water, dried at 50° C. vacuum oven for 4 days, to give a yellow solid, 7.07 g (90.6%), mp 131.5-133° C.

### 4-[(2-Fluorophenyl)-2-(1-Piperazinyl)-Quinazoline Hydrochloride

To a solution of 4-[(2-Fluorophenyl)-2-(1-Piperazinyl)-Quinazoline (5.5 g, 18 mmol) in THF (80 mL) was added a solution of HCl (9.36 mL, 18.7 mmol, 2.0 equiv, 2.0 N in ether, Aldrich) slowly with stirring. Solid formed immediately to give a light yellow paste. Ether (60 mL) was added to dilute the paste. The mixture was stirred at ambient temperature for 2 hr. The light yellow solid was filtered, washed with ether, dried at 50° C. vacuum oven over night, to give light yellow solid 4.52 g (73.5%).

5-HT3A Receptor Binding

Radioligand binding studies can be performed according to Wong, D. T., D. W. Robertson, and L. R. Reid. (1989) Specific 3H-LY-278584 binding to 5-HT3 recognition sites in rat cerebral cortex. European Journal of Pharmacology, 166:1070-110, with some modifications. Briefly, approximately 70 mg/96well plate of frozen cell paste expressing human 5-HT3A receptors was homogenized using a Brinkman Polytron model PT3000 (setting 15,000 rpm, 15 seconds) in 50 mM Tris HCl buffer pH 7.4 containing 2 mM MgCl₂. The homogenate was centrifuged for ten minutes at 40,000 g, washed and recentrifuged. The final pellet was resuspended in 20 mM Tris HCl buffer pH 7.4 at 37 degrees Celsius containing 154 mM NaCl (3.5 mgs/mL). Incubations were initiated by the addition of tissue homogenate to wells of 96 well plates containing ³H-LY-278584 (1 nM, final concentration) and varying concentrations of test compound, buffer or 10 uM MDL-72222 in a final volume of 250 μl. Non-specific binding was defined as the radioactivity remaining in the presence of a saturating concentration of MDL-72222. After a 60 minute incubation at 37° C., assay samples were filtered onto GF/B filtermats presoaked in 0.5% polyethylenimine, using a Skatron cell harvester (Molecular Devices) and washed with ice-cold 50 mM Tris buffer pH 7.4 at 4 degrees Celsius. Radioactivity was quantified by liquid scintillation counting (Betaplate, Wallac Instruments). The IC₅₀ value (concentration at which 50% inhibition of specific binding occurs) was calculated by linear regression of the concentration-response data. Kᵢ values were calculated according to Cheng & Prusoff, where Kᵢ═IC₅₀/(1+(L/K_{d})), where L is the concentration of the radioligand used in the experiment and the K_{d} value is the dissociation constant for the radioligand (determined previously by saturation analysis).

All of the title compounds of the examples were tested and at least one stereoisomer of each such compound exhibited a binding affinity for the human norepinephrine transporter (hNET) receptor, measured as percent inhibition at a concentration of 1 μM, of no less than 50% and up to 100%. At least one stereoisomer of each such compound exhibited a binding affinity for the hNET receptor, measured as percent inhibition at a concentration of 1 μM, of no less than 50% and up to 100%.

The ability of the compounds of this invention to bind to the hNET, hSERT, or the 5HT3 receptor can be determined using conventional radioligand receptor binding assays. The receptors can be heterologously expressed in cell lines and experiments conducted in membrane preparations from those cell lines using procedures. IC₅₀ concentrations can be determined by nonlinear regression of concentration-dependent reduction in specific binding. The Cheng-Prussoff equation can be used to convert the IC₅₀ to Kᵢ concentrations.

hNET Receptor Binding:

Cell pastes of HEK-293 cells transfected with the human norepinephrine transporter were supplied by the Pfizer Ann Arbor Protein Expression and Production group. Pellets were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 sec. Aliquots of membranes (5 mg/ml protein) were stored in liquid nitrogen until used.

The binding assay was set up in Beckman deep-well polypropylene plates with a total volume of 250 μl containing: drug (10⁻⁵M to 10⁻¹²M), cell membranes, and 50 pM [¹²⁵I]-RTI-55 (Perkin Elmer, NEX-272; specific activity 2200 Ci/mmol). The reaction was incubated by gentle agitation for 90 min at room temperature and was terminated by filtration through Whatman GF/C filter plates using a Brandel 96-well plate harvester. Scintillation fluid (100 μl) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to Kᵢ values using the Cheng-Prusoff equation. The Kᵢ values for the hNET are reported below in Table 1.

hSERT Receptor Binding

Cell pastes of HEK-293 cells transfected with the human serotonin transporter were supplied by the Pfizer Ann Arbor Protein Expression and Production Group. Pellets were resuspended in 400 to 700 mL of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at Setting 7 for 30 seconds. Aliquots of membranes (˜2.5 mg/mL protein) were stored in liquid nitrogen until used.

Assays were set up in FlashPlates pre-coated with 0.1% PEI in a total volume of 250 μL containing: drug (10⁻⁵M to 10⁻¹²M), cell membranes, and 50 pM [¹²⁵I]-RTI-55 (Perkin Elmer, NEX-272; specific activity 2200 Ci/mmol). The reaction was incubated and gently agitated for 90 minutes at room temperature, and terminated by removal of assay volume. Plates were covered, and bound [¹²⁵I]-RTI-55 was determined using a Wallac Trilux Beta Plate Counter. Test compounds were run in duplicate, and specific binding was defined as the difference between binding in the presence and absence of 10 μM citalopram.

Excel and GraphPad Prism software were used for data calculation and analysis. IC₅₀ values were converted to Kᵢ values using the Cheng-Prusoff equation. The Kᵢ values for the hSERT are reported below in Table 1.

TABLE 1

| Example | | hNET | hSERT |
|---|---|---|---|
| # | | Ki(nM) | Ki(nM) |
| 1 | 2-(4-Methyl-piperazin-1-yl)-4-phenyl- | 29.7 | |
| | quinazoline | | |
| 2 | 2-(4-Methyl-piperazin-1-yl)-4-p-tolyl- | 77.3 | |
| | quinazoline | | |
| 3 | 4-Phenyl-2-piperazin-1-yl-quinazoline | 4.8 | |
| 4 | 2-(4-Methyl-piperazin-1-yl)-4-o-tolyl- | 34.1 | |
| | quinazoline | | |
| 5 | 2-(3-Methyl-3,9-diaza- | 42.0 | |
| | bicyclo[3.3.1]non-9-yl)-4-phenyl- | | |
| | quinazoline | | |
| 6 | 4-Isopropyl-2-piperazin-1-ylquinazoline | 16.5 | |
| | hydrochloride | | |
| 7 | 2-[1,4]Diazepan-1-yl-4-phenyl- | 2.0 | |
| | quinazoline hydrochloride | | |
| 8 | 2-[1,4]Diazepan-1-yl-4-isopropyl- | 4.8 | |
| | quinazoline hydrochloride | | |
| 9 | 2-(2,5-Dimethyl-piperazin-1-yl)-4- | 22.0 | |
| | phenyl-quinazoline hydrochloride | | |
| 10 | 2-(2,5-Diaza-bicyclo[2.2.1]hept-2-yl)-4- | 3.4 | |
| | phenyl-quinazoline hydrochloride | | |
| 11 | 2-[1-(4-Phenyl-quinazolin-2-yl)- | 80.5 | |
| | piperidin-3-yl]-ethylamine hydrochloride | | |
| 12 | 1-(4-Phenyl-quinazolin-2-yl)-piperidin- | 95.5 | |
| | 4-ylamine hydrochloride | | |
| 13 | N¹-(4-Phenyl-quinazolin-2-yl)-ethane- | 89.2 | |
| | 1,2-diamine hydrochloride | | |
| 14 | 1-(4-Phenyl-quinazolin-2-yl)-pyrrolidin- | 3.7 | |
| | 3-ylamine hydrochloride | | |
| 15 | 2-(2-Methyl-piperazin-1-yl)-4-phenyl- | 4.6 | |
| | quinazoline hydrochloride | | |
| 16 | 1-(4-Phenyl-quinazolin-2-yl)-pyrrolidin- | 31 | |
| | 3-yl amine hydrochloride | | |
| 17 | (4-Phenyl-quinazolin-2-yl)-pyrrolidin-3- | 45 | |
| | yl-amine hydrochloride | | |
| 18 | 4-(2-Fluoro-phenyl)-2-(4-methyl- | 12.0 | |
| | piperazin-1-yl)-quinazoline | | |
| 19 | 4-(2-Chloro-phenyl)-2-(4-methyl- | 26.7 | |
| | piperazin-1-yl)-quinazoline | | |
| 20 | 4-(2-Fluoro-phenyl)-2-piperazin-1-yl- | 1.7 | 12.0 |
| | quinazoline hydrochloride | | |
| 21 | 2-[1,4]Diazepan-1-yl-4-(2-fluoro- | 3.7 | |
| | phenyl)-quinazoline hydrochloride | | |
| 22 | 4-(2-Chloro-phenyl)-2-piperazin-1-yl- | 4.8 | |
| | quinazoline hydrochloride | | |
| 23 | 4-(2-Methoxy-phenyl)-2-piperazin-1-yl- | 29.5 | |
| | quinazoline hydrochloride | | |
| | 4-(2-Methyl-phenyl)-2-(piperazin-1-yl)- | | |
| | quinazoline hydrochloride | | |
| 25 | 4-(4-Fluoro-phenyl)-2-(4-methyl- | 82.0 | |
| | piperazin-1-yl)-quinazoline | | |
| 26 | 4-(3-Fluoro-phenyl)-2-(4-methyl- | 34.9 | |
| | piperazin-1-yl)-quinazoline | | |
| 27 | 2-(4-Methyl-piperazin-1-yl)-4-thiophen- | 67.4 | |
| | 2-yl-quinazoline | | |
| 28 | 4-Benzyl-2-piperazin-1-yl-quinazoline | 93.4 | |
| | hydrochloride | | |
| 29 | 4-(2,6-Difluoro-phenyl)-2-piperazin-4- | 4.8 | |
| | yl-quinazoline | | |
| 30 | 2-(2-Methyl-piperazin-1-yl)-4-phenyl- | 5.5 | |
| | quinazoline | | |
| 31 | 2-(3-Methyl-piperazin-1-yl)-4-phenyl- | 34.5 | |
| | quinazoline | | |
| 32 | 2-(3,9-Diaza-bicyclo[3.3.1]non-3-yl)-4- | 38.5 | |
| | phenyl-quinazoline | | |
| 33 | 2-(3,9-Diaza-bicyclo[3.3.1]non-3-yl)-4- | 19.0 | |
| | (2-fluoro-phenyl)-quinazoline | | |
| 34 | 1-[4-(2-Fluoro-phenyl)-quinazolin-2-yl]- | 3.8 | |
| | pyrrolidin-3-ylamine | | |
| 35 | {1-[4-(2-Fluoro-phenyl)-quinazolin-2- | 19.3 | |
| | yl]-pyrrolidin-3-yl}-methyl-amine | | |
| 36 | 4-(2-Chloro-6-fluoro-phenyl)-2- | 4.0 | |
| | piperazin-1-yl-quinazoline | | |
| 37 | 4-(2,3-Difluoro-phenyl)-2-piperazin-1- | 3.0 | |
| | yl-quinazoline | | |
| 38 | 4-(2,4-Difluoro-phenyl)-2-piperazin-1- | 2.0 | |
| | yl-quinazoline | | |
| 39 | 4-(2-Fluoro-phenyl)-2-(hexahydro- | 41.0 | |
| | pyrrolo[3,4-c]pyrrol-2-yl)-quinazoline | | |
| 40 | 1-[4-(2-Fluoro-phenyl)-quinazolin-2-yl]- | 18.0 | |
| | piperidin-3-ylamine | | |
| 41 | 4-(2-Fluoro-phenyl)-2-piperidin-4-yl- | 4.0 | |
| | quinazoline | | |
| 42 | 4-Phenyl-2-piperidin-4-yl-quinazoline | 13.3 | |
| | hydrochloride | | |
| 43 | 4-(2-Fluoro-phenyl)-2-(1-methyl- | 29.2 | |
| | piperidin-4-yl)-quinazoline | | |
| 44 | 4-(2-Chloro-phenyl)-2-(1-methyl- | 62.5 | |
| | piperidin-4-yl)-quinazoline | | |
| 45 | 4-(2-Chloro-phenyl)-2-piperidin-4-yl- | 8.7 | |
| | quinazoline | | |
| 46 | 4-(2-Methoxy-phenyl)-2-piperidin-4-yl- | 60.3 | |
| | quinazoline | | |
| | 4-(2-Methoxy-phenyl)-2-piperidin-4-yl- | | |
| | quinazoline | | |
| 48 | 4-Phenyl-2-piperidin-3-yl-quinazoline | 48.0 | |
| | 4-Phenyl-2-piperidin-3-yl-quinazoline | | |
| 50 | 4-(4-Phenyl-quinazolin-2-yl)piperidine- | 9.8 | |
| | 4-carboxylic acid methyl ester | | |
| | hydrochloride | | |
| 51 | 4-[4-(2-Fluoro-phenyl)-quinazolin-2-yl]- | 15.1 | |
| | piperidine-4-carboxylic acid methyl ester | | |
| 52 | 3-(4-Phenyl-quinazolin-2-yl)-piperidine- | 53.4 | |
| | 3-carboxylic acid methyl ester | | |
| 53 | 2-Piperazin-1-yl-4-s-tolyl-quinazoline | 10.5 | |
| | hydrochloride | | |
| 54 | 2-(3-Methyl-piperazin-1-yl)-4-phenyl- | 41.0 | |
| | quinazoline | | |
| 55 | 2-(3,9-Diaza-bicyclo[3.3.1]non-9-yl)-4- | 11.0 | |
| | phenyl-quinazoline | | |
| 56 | 2-(3,8-Diaza-bicyclo[3.2.1]oct-8-yl)-4- | 5.0 | |
| | phenyl-quinazoline | | |
| 57 | 2-[1,4]Diazepan-1-yl-4-(2,3-difluoro- | 4.0 | |
| | phenyl)-quinazoline | | |
| 58 | 4-(2,6-Difluoro-phenyl)-quinazolin-2- | 8.0 | |
| | yl]-pyrrolidin-3-yl-amine | | |
| 59 | 7-Fluoro-4-(2-fluoro-phenyl)-2- | 2.7 | |
| | piperazin-1-yl-quinazoline | | |
| 60 | 4-(3-Fluoro-phenyl)-2-piperazin-1-yl- | 4.1 | |
| | quinazoline | | |
| 61 | 4-(2-Chloro-4-fluoro-phenyl)-2- | 6.9 | |
| | piperazin-1-yl-quinazoline | | |
| 62 | 4-(4-Chloro-phenyl)-2-piperazin-1-yl- | 17.3 | |
| | quinazoline | | |
| 63 | 4-(2,6-Dichloro-phenyl)-2-piperazin-1- | 17.6 | |
| | yl-quinazoline | | |
| 64 | 6-Fluoro-4-(2-fluoro-phenyl)-2- | 17.7 | |
| | piperidin-4-yl-quinazoline | | |
| 65 | 7-Fluoro-4-(2-fluoro-phenyl)-2- | 3.3 | |
| | piperidin-4-yl-quinazoline | | |
| 66 | 4-(3-Fluoro-phenyl)-2-piperidin-4-yl- | 5.5 | |
| | quinazoline | | |
| 67 | 4-(3-Fluoro-phenyl)-2-(1-methyl- | 53.5 | |
| | piperidin-4-yl)-quinazoline | | |
| 68 | 4-(4-Fluoro-phenyl)-2-piperidin-4-yl- | 40.6 | |
| | quinazoline | | |
| 69 | 4-(2,6-Difluoro-phenyl)-2-(1-methyl- | 37.9 | |
| | piperidin-4-yl)-quinazoline | | |
| 70 | 4-(2,6-Difluoro-phenyl)-2-piperidin-4-yl- | 2.9 | |
| | quinazoline | | |
| 71 | 4-(2,3-Difluoro-phenyl)-2-piperidin-4-yl- | 3.0 | |
| | quinazoline | | |
| 72 | 4-(2,4-Difluoro-phenyl)-2-piperidin-4-yl- | 17.7 | |
| | quinazoline | | |
| 73 | 2-Piperidin-4-yl-4-(2,3,6-trifluoro- | 15.9 | |
| | phenyl)-quinazoline | | |
| 74 | 4-(2-Chloro-6-fluoro-phenyl)-2-(1- | 22.9 | |
| | methyl-piperidin-4-yl)-quinazoline | | |
| 75 | 4-(2-Chloro-6-fluoro-phenyl)-2- | 5.9 | |
| | piperidin-4-yl-quinazoline | | |
| 76 | 2-Piperidin-4-yl-4-o-tolyl-quinazoline | 44.7 | |
| 77 | 4-(2-Fluoro-phenyl)-2-piperidin-3-yl- | 22.8 | |
| | quinazoline | | |
| 78 | 2-Piperidin-3-yl-4-o-tolyl-quinazoline | 19.8 | |
| 79 | 4-(2-Fluoro-phenyl)-2-(4-phenyl- | 63.4 | |
| | piperidin-4-yl)-quinazoline | | |
| 80 | 2-(2,5-Diaza-bicyclo[2.2.1]hept-2-yl)-4- | 9.5 | |
| | phenyl-quinazoline | | |
| 81 | 2-(Hexahydro-pyrrolo[3,4-c]pyrrol-2-yl)- | 61.0 | |
| | 4-phenyl-quinazoline | | |
| 82 | 4-(2,4-Difluoro-phenyl)-2-(2-methyl- | 19.4 | 64.1 |
| | piperazin-1-yl)-quinazoline | | |
| 83 | 4-(2,6-Difluoro-phenyl)-7-fluoro-2-(4- | 37.6 | 314 |
| | methyl-piperazin-1-yl)-quinazoline | | |
| 84 | [4-(2,6-Difluoro-phenyl)-7-fluoro- | 49.8 | 653 |
| | quinazolin-2-yl]-pyrrolidin-3-yl-amine | | |
| 85 | 4-(2-Chloro-6-fluoro-phenyl)-2-(2- | 26.3 | 265 |
| | methyl-piperazin-1-yl)-quinazoline | | |
| 86 | 4-(2,6-Difluoro-phenyl)-2-(2-methyl- | 14.4 | 88.6 |
| | piperazin-1-yl)-quinazoline | | |
| 87 | 4-(2,3-Difluoro-phenyl)-2-(2-methyl- | 20.0 | 99.1 |
| | piperazin-1-yl)-quinazoline | | |
| 88 | 4-(2,3-Difluoro-phenyl)-2-(2-methyl- | 18.9 | 69.3 |
| | piperazin-1-yl)-quinazoline | | |
| 89 | 7-Chloro-4-phenyl-2-piperazin-1-yl- | 49.0 | 110 |
| | quinazoline | | |
| 90 | 4-(3-methoxy-phenyl)-2-piperazin-1-yl- | 14.6 | 152 |
| | quinazoline | | |
| 91 | 6-Bromo-4-phenyl-2-piperazin-1-yl- | 211 | 31.4 |
| | quinazoline | | |
| 92 | 6-Fluoro-4-phenyl-2-piperazin-1-yl- | 33.8 | 53 |
| | quinazoline | | |
| 93 | 1-[4-(2,6-Difluoro-phenyl)-7-fluoro- | 21.9 | 50.6 |
| | quinazolin-2-yl]-pyrrolidin-3-ylamine | | |
| 94 | 1-[4-(2,6-Difluoro-phenyl)-7-fluoro- | 23.7 | 15.7 |
| | quinazolin-2-yl]-pyrrolidin-3-ylamine | | |
| 95 | 1-[4-(2,6-Difluoro-phenyl)-quinazolin-2- | 76.9 | 322 |
| | yl]-pyrrolidin-3-ylamine | | |
| 96 | 1-[4-(2,6-Difluoro-phenyl)-quinazolin-2- | 12.2 | 70.8 |
| | yl]-pyrrolidin-3-ylamine | | |
| 97 | 7-Fluoro-4-phenyl-2-piperazin-1-yl- | 13.8 | 49.6 |
| | quinazoline | | |
| 98 | 4-(2,6-Difluoro-phenyl)-7-fluoro-2- | 20 | 32 |
| | piperazin-1-yl-quinazoline | | |
| 99 | [4-(2,6-Difluoro-phenyl)-quinazolin-2- | 45.7 | 2900 |
| | yl]-pyrrolidin-3-yl-amine | | |
| 100 | [4-(2,6-Difluoro-phenyl)-7-fluoro- | 29.3 | 649 |
| | quinazolin-2-yl]-pyrrolidin-3-yl-amine | | |
| 101 | [4-(2,6-Difluoro-phenyl)-quinazolin-2- | 7.5 | 4870 |
| | yl]-pyrrolidin-3-yl-amine | | |
| 102 | 1-[4-(2,3-Difluoro-phenyl)-quinazolin-2- | 11.4 | 155 |
| | yl]-pyrrolidin-3-ylamine | | |
| 103 | 4-(3,4-Difluoro-phenyl)-2-(4-methyl- | 101 | 645 |
| | piperazin-1-yl)-quinazoline | | |
| 104 | 4-(2,6-Difluoro-phenyl)-7-fluoro-2-(2- | 36.4 | 27.6 |
| | methyl-piperazin-1-yl)-quinazoline | | |
| 105 | 7-Fluoro-2-(2-methyl-piperazin-1-yl)-4- | 16.6 | 65.0 |
| | phenyl-quinazoline | | |
| 106 | 7-Fluoro-2-(2-methyl-piperazin-1-yl)-4- | 18.3 | 49.7 |
| | phenyl-quinazoline | | |
| 107 | 4-(2,6-Difluoro-phenyl)-7-fluoro-2-(2- | 47.3 | 30.8 |
| | methyl-piperazin-1-yl)-quinazoline | | |
| 108 | 4-(3,4-difluoro-phenyl)-2-piperazin-1-yl- | 7.7 | 77.7 |
| | quinazoline | | |
| 109 | 1-[4-(2,3-Difluoro-phenyl)-7-fluoro- | 13.9 | 30.4 |
| | quinazolin-2-yl]-pyrrolidin-3(S)-yl- | | |
| | amine | | |
| 110 | 1-[4-(2,3-Difluoro-phenyl)-7-fluoro- | 13.6 | 57.1 |
| | quinazolin-2-yl]-pyrrolidin-3(R)-yl- | | |
| | amine | | |
| 111 | 4-(2,3-Difluoro-phenyl)-7-fluoro-2- | 18.6 | 37.1 |
| | piperazin-1-yl-quinazoline | | |
| 112 | 4-(3,4-Difluoro-phenyl)-7-fluoro-2-(4- | 707 | 204 |
| | methyl-piperazin-1-yl)-quinazoline | | |
| 113 | 4-(3-chloro-phenyl)-2-piperazin-1-yl- | 28.6 | 119 |
| | quinazoline | | |
| 114 | 4-(3,4-Difluoro-phenyl)-2-(4-methyl- | 31.7 | 1090 |
| | piperazin-1-yl)-quinazoline | | |
| 115 | 4-(3,4-dichloro-phenyl)-2-piperazin-1-yl- | 114 | 332 |
| | quinazoline | | |
| 116 | [4-(2,3-Difluoro-phenyl)-7-fluoro- | 138 | 963 |
| | quinazolin-2-yl]-pyrrolidin-3-yl-amine | | |
| 117 | 7-Fluoro-4-(4-fluoro-2-methyl-phenyl)- | 32.9 | 114 |
| | 2-piperazin-1-yl-quinazoline | | |
| 118 | 7-Chloro-4-(4-fluoro-2-methyl-phenyl)- | 67.2 | 49 |
| | 2-piperazin-1-yl-quinazoline | | |
| 119 | 4-(3,4-Difluoro-phenyl)-7-fluoro-2- | 7.3 | 22.7 |
| | piperazin-1-yl-quinazoline | | |
| 120 | 4-(2,4-Dichloro-phenyl)-7-fluoro-2- | 95 | 34.1 |
| | piperazin-1-yl-quinazoline | | |
| 121 | 4-(2,3-Difluoro-phenyl)-6-fluoro-2- | 15.7 | 32.5 |
| | piperazin-1-yl-quinazoline | | |
| 122 | 4-(2,4-Difluoro-phenyl)-6-fluoro-2- | 12.1 | 15.5 |
| | piperazin-1-yl-quinazoline | | |
| 123 | 4-(2,3-Difluoro-phenyl)-6,7-difluoro-2- | 28.5 | 47.2 |
| | piperazin-1-yl-quinazoline | | |
| 124 | 4-(2,3-Difluoro-phenyl)-7-fluoro-2-(2- | 21.6 | 28.1 |
| | methyl-piperazin-1-yl)-quinazoline | | |
| 125 | 4-(2,5-dichloro-phenyl)-2-piperazin-1-yl- | 32.2 | 636 |
| | quinazoline | | |
| 126 | 4-(3,5-difluoro-phenyl)-2-piperazin-1-yl- | 3.7 | 107 |
| | quinazoline | | |
| 127 | 4-(2,6-Difluoro-phenyl)-2-(2-methyl- | 33.9 | 58.6 |
| | piperazin-1-yl)-quinazoline | | |
| 128 | 6-Chloro-4-phenyl-2-piperazin-1-yl- | 35.5 | 11.4 |
| | quinazoline | | |
| 129 | 4-(2-Fluoro-phenyl)-6-chloro-2- | 25.6 | 8.4 |
| | piperazin-1-yl-quinazoline | | |
| 130 | 4-(2,3-Difluoro-phenyl)-7-fluoro-2-(2- | 24.7 | 44.3 |
| | methyl-piperazin-1-yl)-quinazoline | | |
| 131 | 4-(2,6-Difluoro-phenyl)-6-chloro-2- | 30 | 4.8 |
| | piperazin-1-yl-quinazoline | | |
| 132 | N1-[4-(2,4-Difluoro-phenyl)-quinazolin- | | |
| | 2-yl]-ethane-1,2-diamine | | |
| 133 | 8-Fluoro-4-phenyl-2-piperazin-1-yl- | 22.0 | 1590 |
| | quinazoline | | |
| 134 | 4-(2-Chloro-4-fluoro-phenyl)-7-fluoro-2- | 30.3 | 62.6 |
| | piperazin-1-yl-quinazoline | | |
| 135 | 7-Fluoro-2-piperazin-1-yl-4-thiazol-2-yl- | 56.8 | 313 |
| | quinazoline | | |
| 136 | 4-(2,-Methoxy-phenyl)-7-fluoro-2- | 43.1 | 76.5 |
| | piperazin-1-yl-quinazoline | | |
| 137 | 7-Fluoro-4-(5-fluoro-2-methyl-phenyl)- | 16.6 | 190 |
| | 2-piperazin-1-yl-quinazoline | | |
| 138 | 4-(2,4-Difluoro-phenyl)-7-fluoro-2-(2- | 35.4 | 24.6 |
| | methyl-piperazin-1-yl)-quinazoline | | |
| 139 | 4-(2,4-Difluoro-phenyl)-7-fluoro-2-(2- | 30.7 | 24.9 |
| | methyl-piperazin-1-yl)-quinazoline | | |
| 140 | 4-(2,4-Difluoro-phenyl)-7-fluoro-2- | 28.3 | 16.7 |
| | piperazin-1-yl-quinazoline | | |
| 141 | Azetidin-3-yl-[4-(2,4-difluoro-phenyl)-7- | 102 | 1920 |
| | fluoro-quinazolin-2-yl]-amine | | |
| 142 | 4-(2,4-Difluoro-phenyl)-7-fluoro-2- | 90.6 | 91.4 |
| | (hexahydro-pyrrolo[3,4-b]pyrrol-1-yl)- | | |
| | quinazoline | | |
| 143 | [4-(2,4-Difluoro-phenyl)-7-fluoro- | 17.9 | 9.3 |
| | quinazolin-2-yl]-pyrrolidin-3-yl-amine | | |
| 144 | [4-(2,4-Difluoro-phenyl)-7-fluoro- | 23.4 | 26.5 |
| | quinazolin-2-yl]-pyrrolidin-3-yl-amine | | |
| 145 | 5-Methyl-4-phenyl-2-piperazin-1-yl- | 16.6 | 121 |
| | quinazoline | | |
| 146 | 4-(2,6-Difluoro-phenyl)-6,7-difluoro-2- | 31.5 | 19.0 |
| | piperazin-1-yl-quinazoline | | |
| 147 | 4-(2,4-Difluoro-phenyl)-6,7-difluoro-2- | 28.0 | 25.6 |
| | piperazin-1-yl-quinazoline | | |
| 148 | [4-(2,6-Difluoro-phenyl)-quinazolin-2- | 267 | 4060 |
| | yl]-piperidin-4-yl-amine | | |
| 149 | {1-[4-(2,6-Difluoro-phenyl)-quinazolin- | 135 | 1620 |
| | 2-yl]-pyrrolidin-3-yl}-methyl-amine | | |
| 150 | {1-[4-(2,4-Difluoro-phenyl)-quinazolin- | 41.5 | 2700 |
| | 2-yl]-pyrrolidin-3-yl}-methyl-amine | | |
| 151 | {1-[4-(2,6-Difluoro-phenyl)-quinazolin- | 384 | 5540 |
| | 2-yl]-piperidin-4-yl}-methyl-amine | | |
| 152 | N1-[4-(2,4-Difluoro-phenyl)-quinazolin- | 25.8 | 10000 |
| | 2-yl]-propane-1,3-diamine | | |
| 153 | {1-[4-(2,4-Difluoro-phenyl)-quinazolin- | 150 | 10000 |
| | 2-yl]-piperidin-4-yl}-methyl-amine | | |
| 154 | [4-(2,4-Difluoro-phenyl)-quinazolin-2- | 58.1 | 5370 |
| | yl]-piperidin-4-yl-amine | | |
| 155 | [4-(2,4-Difluoro-phenyl)-quinazolin-2- | 425 | 10000 |
| | yl]-pyrrolidin-3-ylmethyl-amine | | |
| 156 | [4-(2,3-Difluoro-phenyl)-quinazolin-2- | 221 | 6980 |
| | yl]-piperidin-4-yl-amine | | |
| 157 | {1-[4-(2,3-Difluoro-phenyl)-quinazolin- | 148 | 4250 |
| | 2-yl]-pyrrolidin-3-yl}-methyl-amine | | |
| 158 | {1-[4-(2,3-Difluoro-phenyl)-quinazolin- | 207 | 6030 |
| | 2-yl]-piperidin-4-yl}-methyl-amine | | |
| 159 | N1-[4-(2,3-Difluoro-phenyl)-quinazolin- | 123 | 10000 |
| | 2-yl]-propane-1,3-diamine | | |
| 160 | 7-Fluoro-2-piperazin-1-yl-4-(2- | 60.7 | 976 |
| | trifluoromethyl-phenyl)-quinazoline | | |
| 161 | 2-(2,4-Difluoro-phenyl)-4-piperazin-1- | 3480 | 382 |
| | yl-quinazoline | | |
| 162 | 4-(2,6-Difluoro-phenyl)-7-fluoro-2- | 11.4 | 52.8 |
| | piperidin-4-yl-quinazoline | | |
| 163 | 7,8-Difluoro-4-phenyl-2-piperazin-1-yl- | 12.6 | 441 |
| | quinazoline | | |
| 164 | 4-(2,6-Difluoro-phenyl)-2-(2-ethyl- | 31.8 | 18.0 |
| | piperazin-1-yl)-quinazoline | | |
| 165 | 4-(2,4-Difluoro-phenyl)-2-(2-ethyl- | 31 | 14.4 |
| | piperazin-1-yl)-quinazoline | | |
| 166 | 4-(2,3-Difluoro-phenyl)-2-(2-isopropyl- | 79.7 | 185 |
| | piperazin-1-yl)-quinazoline | | |
| 167 | 4-(2,4-Difluoro-phenyl)-2-(2-isopropyl- | 66.1 | 68.8 |
| | piperazin-1-yl)-quinazoline | | |

1. A compound having the formula 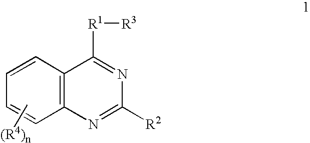
or a pharmaceutically acceptable salt, solvate or hydrate thereof, wherein
R¹ is (C₁-C₆)alkyl, (C₃-C₈)cycloalkyl, (C₁-C₆)alkoxy, aryl, amino, halogen, hydroxy, heteroaryl, or a saturated, unsaturated, or aromatic five to seven membered monocyclic heterocyclic ring containing from one to three heteroatoms independently selected from oxygen, nitrogen, and sulfur;
R² is (C₁-C₆)alkyl, (C₃-C₈)cycloalkyl, amino, or a saturated, unsaturated, or aromatic five to seven membered monocyclic heterocyclic ring or a six to ten membered bicyclic ring containing from one to three heteroatoms independently selected from oxygen, nitrogen, and sulfur, any of which may be unsubstituted or substituted with one or more of the following substituents: (C₁-C₆)alkyl, substituted (C₁-C₆)alkyl, amino, (C₁-C₆)alkylamino, or a heterocyclic group;
R³ is independently selected from one or more of hydrogen, (C₁-C₆)alkyl, (C₃-C₈)cycloalkyl, (C₁-C₆)alkoxy, aryl, amino, halogen, or hydroxy groups;
R⁴ is independently selected from one or more of hydrogen, halogen, —NO₂, (C₁-C₆)alkyl, (C₁-C₆)alkoxy, or a heterocyclic group, wherein each occurrence of R⁴ may be the same or different; and n is 0, 1, 2, or 3.

2. The compound of claim 1, wherein R¹ is aryl and R² is piperazinyl or piperidinyl.

3. The compound of claim 2, wherein R¹ is a substituted phenyl group.

4. The compound of claim 1 having the structure 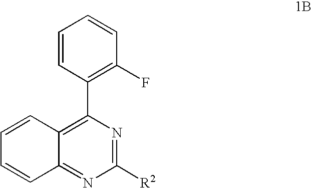
wherein R² is substituted or unsubstituted piperidinyl, homopiperazinyl, piperazinyl, or 3-aminopyrrolidinyl and the pharmaceutically acceptable salts thereof.

5. The compound of claim 1, wherein said compound is selected from the group consisting of:
2-(4-Methyl-piperazin-1-yl)-4-phenyl-quinazoline;
2-(4-Methyl-piperazin-1-yl)-4-p-tolyl-quinazoline;
4-Phenyl-2-piperazin-1-yl-quinazoline;
2-(4-Methyl-piperazin-1-yl)-4-o-tolyl-quinazoline;
2-(3-Methyl-3,9-diaza-bicyclo[3.3.1]non-9-yl)-4-phenyl-quinazoline;
4-Isopropyl-2-piperazin-1-yl-quinazoline;
2-[1,4]Diazepan-1-yl-4-phenyl-quinazoline;
2-[1,4]Diazepan-1-yl-4-isopropyl-quinazoline;
2-(2,5-Dimethyl-piperazin-1-yl)-4-phenyl-quinazoline;
2-(2,5-Diaza-bicyclo[2.2.1 ]hept-2-yl)-4-phenyl-quinazoline;
2-[1-(4-Phenyl-quinazolin-2-yl)-piperidin-3-yl]-ethylamine;
1-(4-Phenyl-quinazolin-2-yl)-piperidin-4-ylamine;
N1-(4-Phenyl-quinazolin-2-yl)-ethane-1,2-diamine;
1-(4-Phenyl-quinazolin-2-yl)-pyrrolidin-3-ylamine;
2-(2-Methyl-piperazin-1-yl)-4-phenyl-quinazoline;
1-(4-Phenyl-quinazolin-2-yl)-pyrrolidin-3-yl amine;
3-(4-Phenyl-quinazolin-2-yl)-aminopyrrolidine;
4-(2-Fluoro-phenyl)-2-(4-methyl-piperazin-1-yl)-quinazoline;
4-(2-Chloro-phenyl)-2-(4-methyl-piperazin-1-yl)-quinazoline;
4-(2-Fluoro-phenyl)-2-piperazin-1-yl-quinazoline;
2-[1,4]Diazepan-1-yl-4-(2-fluoro-phenyl)-quinazoline;
4-(2-Chloro-phenyl)-2-piperazin-1-yl-quinazoline;
4-(2-Methoxy-phenyl)-2-piperazin-1-yl-quinazoline;
4-(2-Methyl-phenyl-2-piperazin-1-yl)quinazoline;
4-(4-Fluoro-phenyl)-2-(4-methyl-piperazin-1-yl)-quinazoline;
4-(3-Fluoro-phenyl)-2-(4-methyl-piperazin-1-yl)-quinazoline;
2-(4-Methyl-piperazin-1-yl)-4-thiophen-2-yl-quinazoline;
4-Benzyl-2-piperazin-1-yl-quinazoline;
4-(2,6-Difluoro-phenyl)-2-piperazin-4-yl-quinazoline;
(R)-(−)-2-(2-Methyl-piperazin-1-yl)-4-phenyl-quinazoline;
(R)-(+)-2-(3-Methyl-piperazin-1-yl)-4-phenyl-quinazoline;
2-(3,9-Diaza-bicyclo[3.3.1]non-3-yl)-4-phenyl-quinazoline;
2-(3,9-Diaza-bicyclo[3.3.1]non-3-yl)-4-(2-fluoro-phenyl)-quinazoline;
(S)-(+)-1-[4-(2-Fluoro-phenyl)-quinazolin-2-yl]-pyrrolidin-3-yl amine;
(S)-(+)-{1-[4-(2-Fluoro-phenyl)-quinazolin-2-yl]-pyrrolidin-3-yl}-methyl-amine;
4-(2-Chloro-6-fluoro-phenyl)-2-piperazin-1-yl-quinazoline;
4-(2,3-Difluoro-phenyl)-2-piperazin-1-yl-quinazoline;
4-(2,4-Difluoro-phenyl)-2-piperazin-1-yl-quinazoline;
4-(2-Fluoro-phenyl)-2-(hexahydro-pyrrolo[3,4-c]pyrrol-2-yl)-quinazoline;
(S)-(+)-1-[4-(2-Fluoro-phenyl)-quinazolin-2-yl]-piperidin-3-ylamine;
4-(2-Fluoro-phenyl)-2-(piperidin-4-yl)-quinazoline;
4-Phenyl-2-piperidin-4-yl-quinazoline;
4-(2-Fluoro-phenyl)-2-(1-methyl-piperidin-4-yl)-quinazoline;
4-(2-Chloro-phenyl)-2-(1-methyl-piperidin-4-yl)-quinazoline;
4-(2-Chloro-phenyl)-2-piperidin-4-yl-quinazoline;
4-(2-Methoxy-phenyl)-2-piperidin-4-yl-quinazoline;
4-(2-Methyl-phenyl)-2-piperidin-4-yl-quinazoline;
4-Phenyl-2-piperidin-3-yl-quinazoline;
4-(4-Phenyl-quinazolin-2-yl)piperidine-4-carboxylic acid methyl ester;
4-[4-(2-Fluoro-phenyl)-quinazolin-2-yl]-piperidine-4-carboxylic acid methyl ester;
3-(4-Phenyl-quinazolin-2-yl)-piperidine-3-carboxylic acid methyl ester;
2-Piperazin-1-yl-4-s-tolyl-quinazoline;
2-(3-Methyl-piperazin-1-yl)-4-phenyl-quinazoline;
2-(3,9-Diaza-bicyclo[3.3.1]non-9-yl)-4-phenyl-quinazoline;
2-(3,8-Diaza-bicyclo[3.2.1]oct-8-yl)-4-phenyl-quinazoline;
2-[1,4]Diazepan-1-yl-4-(2,3-difluoro-phenyl)-quinazoline;
4-(2,6-Difluoro-phenyl)-quinazolin-2-yl]-pyrrolidin-3-yl-amine;
7-Fluoro-4-(2-fluoro-phenyl)-2-piperazin-1-yl-quinazoline;
4-(3-Fluoro-phenyl)-2-piperazin-1-yl-quinazoline;
4-(2-Chloro-4-fluoro-phenyl)-2-piperazin-1-yl-quinazoline;
4-(4-Chloro-phenyl)-2-piperazin-1-yl-quinazoline;
4-(2,6-Dichloro-phenyl)-2-piperazin-1-yl-quinazoline;
6-Fluoro-4-(2-fluoro-phenyl)-2-piperidin-4-yl-quinazoline;
7-Fluoro4-(2-fluoro-phenyl)-2-piperidin-4-yl-quinazoline;
4-(3-Fluoro-phenyl)-2-piperidin-4-yl-quinazoline;
4-(3-Fluoro-phenyl)-2-(1-methyl-piperidin-4-yl)-quinazoline;
4-(4-Fluoro-phenyl)-2-piperidin-4-yl-quinazoline;
4-(2,6-Difluoro-phenyl)-2-(1-methyl-piperidin-4-yl)-quinazoline;
4-(2,6-Difluoro-phenyl)-2-piperidin-4-yl-quinazoline;
4-(2,3-Difluoro-phenyl)-2-piperidin-4-yl-quinazoline;
4-(2,4-Difluoro-phenyl)-2-piperidin-4-yl-quinazoline;
2-Piperidin-4-yl-4-(2,3,6-trifluoro-phenyl)-quinazoline;
4-(2-Chloro-6-fluoro-phenyl)-2-(1-methyl-piperidin-4-yl)-quinazoline;
4-(2-Chloro-6-fluoro-phenyl)-2-piperidin-4-yl-quinazoline;
2-Piperidin-4-yl-4-o-tolyl-quinazoline;
4-(2-Fluoro-phenyl)-2-piperidin-3-yl-quinazoline;
2-Piperidin-3-yl-4-o-tolyl-quinazoline;
4-(2-Fluoro-phenyl)-2-(4-phenyl-piperidin-4-yl)-quinazoline;
2-(2,5-Diaza-bicyclo[2.2.1]hept-2-yl)-4-phenyl-quinazoline;
2-(Hexahydro-pyrrolo[3,4-c]pyrrol-2-yl)-4-phenyl-quinazoline;
4-(2,4-Difluoro-phenyl)-2-(2-methyl-piperazin-1-yl)-quinazoline;
4-(2,6-Difluoro-phenyl)-7-fluoro-2-(4-methyl-piperazin-1-yl)-quinazoline;
[4-(2,6-Difluoro-phenyl)-7-fluoro-quinazolin-2-yl]-pyrrolidin-3-yl-amine;
4-(2-Chloro-6-fluoro-phenyl)-2-(2-methyl-piperazin-1-yl)-quinazoline;
4-(2,6-Difluoro-phenyl)-2-(2-methyl-piperazin-1-yl)-quinazoline;
4-(2,3-Difluoro-phenyl)-2-(2-methyl-piperazin-1-yl)-quinazoline;
4-(2,3-Difluoro-phenyl)-2-(2-methyl-piperazin-1-yl)-quinazoline;
7-Chloro-4-phenyl-2-piperazin-1-yl-quinazoline;
4-(3-methoxy-phenyl)-2-piperazin-1-yl-quinazoline;
6-Bromo-4-phenyl-2-piperazin-1-yl-quinazoline;
6-Fluoro-4-phenyl-2-piperazin-1-yl-quinazoline;
1-[4-(2,6-Difluoro-phenyl)-7-fluoro-quinazolin-2-yl]-pyrrolidin-3-ylamine;
1-[4-(2,6-Difluoro-phenyl)-7-fluoro-quinazolin-2-yl-pyrrolidin-3-ylamine;
1-[4-(2,6-Difluoro-phenyl)-quinazolin-2-yl]-pyrrolidin-3-ylamine;
1-[4-(2,6-Difluoro-phenyl)-quinazolin-2-yl]-pyrrolidin-3-ylamine;
7-Fluoro-4-phenyl-2-piperazin-1-yl-quinazoline;
4-(2,6-Difluoro-phenyl)-7-fluoro-2-piperazin-1-yl-quinazoline;
[4-(2,6-Difluoro-phenyl)-quinazolin-2-yl]-pyrrolidin-3-yl-amine;
[4-(2,6-Difluoro-phenyl)-7-fluoro-quinazolin-2-yl]-pyrrolidin-3-yl-amine;
[4-(2,6-Difluoro-phenyl)-quinazolin-2-yl]-pyrrolidin-3-yl-amine;
1-[4-(2,3-Difluoro-phenyl)-quinazolin-2-yl]-pyrrolidin-3-ylamine;
4-(3,4-Difluoro-phenyl)-2-(4-methyl-piperazin-1-yl)-quinazoline;
4-(2,6-Difluoro-phenyl)-7-fluoro-2-(2-methyl-piperazin-1-yl)-quinazoline;
7-Fluoro-2-(2-methyl-piperazin-1-yl)-4-phenyl-quinazoline;
7-Fluoro-2-(2-methyl-piperazin-1-yl)-4-phenyl-quinazoline;
4-(2,6-Difluoro-phenyl)-7-fluoro-2-(2-methyl-piperazin-1-yl)-quinazoline;
4-(3,4-difluoro-phenyl)-2-piperazin-1-yl-quinazoline;
1-[4-(2,3-Difluoro-phenyl)-7-fluoro-quinazolin-2-yl]-pyrrolidin-3(S)-yl-amine;
1-[4-(2,3-Difluoro-phenyl)-7-fluoro-quinazolin-2-yl]-pyrrolidin-3(R)-yl-amine;
4-(2,3-Difluoro-phenyl)-7-fluoro-2-piperazin-1-yl-quinazoline;
4-(3,4-Difluoro-phenyl)-7-fluoro-2-(4-methyl-piperazin-1-yl)-quinazoline;
4-(3-chloro-phenyl)-2-piperazin-1-yl-quinazoline;
4-(3,4-Difluoro-phenyl)-2-(4-methyl-piperazin-1-yl)-quinazoline;
4-(3,4-dichloro-phenyl)-2-piperazin-1-yl-quinazoline;
[4-(2,3-Difluoro-phenyl)-7-fluoro-quinazolin-2-yl]-pyrrolidin-3-yl-amine;
7-Fluoro-4-(4-fluoro-2-methyl-phenyl)-2-piperazin-1-yl-quinazoline;
7-Chloro-4-(4-fluoro-2-methyl-phenyl)-2-piperazin-1-yl-quinazoline;
4-(3,4-Difluoro-phenyl)-7-fluoro-2-piperazin-1-yl-quinazoline;
4-(2,4-Dichloro-phenyl)-7-fluoro-2-piperazin-1-yl-quinazoline;
4-(2,3-Difluoro-phenyl)-6-fluoro-2-piperazin-1-yl-quinazoline;
4-(2,4-Difluoro-phenyl)-6-fluoro-2-piperazin-1-yl-quinazoline;
4-(2,3-Difluoro-phenyl)-6,7-difluoro-2-piperazin-1-yl-quinazoline;
4-(2,3-Difluoro-phenyl)-7-fluoro-2-(2-methyl-piperazin-1-yl)-quinazoline;
4-(2,5-dichloro-phenyl)-2-piperazin-1-yl-quinazoline;
4-(3,5-difluoro-phenyl)-2-piperazin-1-yl-quinazoline;
4-(2,6-Difluoro-phenyl)-2-(2-methyl-piperazin-1-yl)-quinazoline;
6-Chloro-4-phenyl-2-piperazin-1-yl-quinazoline;
4-(2-Fluoro-phenyl)-6-chloro-2-piperazin-1-yl-quinazoline;
4-(2,3-Difluoro-phenyl)-7-fluoro-2-(2-methyl-piperazin-1-yl)-quinazoline;
4-(2,6-Difluoro-phenyl)-6-chloro-2-piperazin-1-yl-quinazoline;
N1-[4-(2,4-Difluoro-phenyl)-quinazolin-2-yl]-ethane-1,2-diamine;
8-Fluoro-4-phenyl-2-piperazin-1-yl-quinazoline;
4-(2-Chloro-4-fluoro-phenyl)-7-fluoro-2-piperazin-1-yl-quinazoline;
7-Fluoro-2-piperazin-1-yl-4-thiazol-2-yl-quinazoline;
4-(2,-Methoxy-phenyl)-7-fluoro-2-piperazin-1-yl-quinazoline;
7-Fluoro-4-(5-fluoro-2-methyl-phenyl)-2-piperazin-1-yl-quinazoline;
4-(2,4-Difluoro-phenyl)-7-fluoro-2-(2-methyl-piperazin-1-yl)-quinazoline;
4-(2,4-Difluoro-phenyl)-7-fluoro-2-(2-methyl-piperazin-1-yl)-quinazoline;
4-(2,4-Difluoro-phenyl)-7-fluoro-2-piperazin-1-yl-quinazoline;
Azetidin-3-yl-[4-(2,4-difluoro-phenyl)-7-fluoro-quinazolin-2-yl]-amine;
4-(2,4-Difluoro-phenyl)-7-fluoro-2-(hexahydro-pyrrolo[3,4-b]pyrrol-1-yl)-quinazoline;
[4-(2,4-Difluoro-phenyl)-7-fluoro-quinazolin-2-yl]-pyrrolidin-3-yl-amine;
[4-(2,4-Difluoro-phenyl)-7-fluoro-quinazolin-2-yl]-pyrrolidin-3-yl-amine;
5-Methyl-4-phenyl-2-piperazin-1-yl-quinazoline;
4-(2,6-Difluoro-phenyl)-6,7-difluoro-2-piperazin-1-yl-quinazoline;
4-(2,4-Difluoro-phenyl)-6,7-difluoro-2-piperazin-1-yl-quinazoline;
[4-(2,6-Difluoro-phenyl)-quinazolin-2-yl]-piperidin-4-yl-amine;
{1-[4-(2,6-Difluoro-phenyl)-quinazolin-2-yl]-pyrrolidin-3-yl}-methyl-amine;
{1-[4-(2,4-Difluoro-phenyl)-quinazolin-2-yl]-pyrrolidin-3-yl}-methyl-amine;
{1-[4-(2,6-Difluoro-phenyl)-quinazolin-2-yl]-piperidin-4-yl}-methyl-amine;
N1-[4-(2,4-Difluoro-phenyl)-quinazolin-2-yl]-propane-1,3-diamine;
{1-[4-(2,4-Difluoro-phenyl)-quinazolin-2-yl]-piperidin-4-yl}-methyl-amine;
[4-(2,4-Difluoro-phenyl)-quinazolin-2-yl]-piperidin-4-yl-amine;
[4-(2,4-Difluoro-phenyl)-quinazolin-2-yl]-pyrrolidin-3-ylmethyl-amine;
[4-(2,3-Difluoro-phenyl)-quinazolin-2-yl]-piperidin-4-yl-amine;
{1-[4-(2,3-Difluoro-phenyl)-quinazolin-2-yl]-pyrrolidin-3-yl}-methyl-amine;
{1-[4-(2,3-Difluoro-phenyl)-quinazolin-2-yl]-piperidin-4-yl}-methyl-amine;
N1-[4-(2,3-Difluoro-phenyl)-quinazolin-2-yl]-propane-1,3-diamine;
7-Fluoro-2-piperazin-1-yl-4-(2-trifluoromethyl-phenyl)-quinazoline;
2-(2,4-Difluoro-phenyl)-4-piperazin-1-yl-quinazoline;
4-(2,6-Difluoro-phenyl)-7-fluoro-2-piperidin-4-yl-quinazoline;
7,8-Difluoro-4-phenyl-2-piperazin-1-yl-quinazoline;
4-(2,6-Difluoro-phenyl)-2-(2-ethyl-piperazin-1-yl)-quinazoline;
4-(2,4-Difluoro-phenyl)-2-(2-ethyl-piperazin-1-yl)-quinazoline;
4-(2,3-Difluoro-phenyl)-2-(2-isopropyl-piperazin-1-yl)-quinazoline;
4-(2,4-Difluoro-phenyl)-2-(2-isopropyl-piperazin-1-yl)-quinazoline; and pharmaceutically acceptable salts, solvates, and hydrates thereof.

6. A pharmaceutical composition comprising the compound of claim 1 or a pharmaceutically acceptable salt thereof; and a pharmaceutically acceptable carrier.

7. A method of treating a disorder or condition selected from the group consisting of norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression or reactive depression including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); age related cognitive deficit disruptive behavior disorder; behavioral disturbances associated with mental retardation, autistic disorder, and conduct disorder; anxiety disorders such as panic disorder with or without agoraphobia, agoraphobia without history of panic disorder, specific phobias including specific animal phobias, social anxiety, social phobia including social anxiety disorder, obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders including schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions including dependencies on, or addictions to, alcohol, heroin, cocaine, benzodiazepines, psychoactive substances, nicotine, or phenobarbitol and behavioral addictions including addiction to gambling; ocular disorders such as glaucoma and ischemic retinopathy addictive disorders including those due to alcohol, nicotine, and other psychoactive substances and withdrawal syndrome, adjustment disorders and disruptive behavior disorders including depressed mood, anxiety, mixed anxiety and depressed mood, disturbance of conduct, and mixed disturbance of conduct and mood; age-associated learning and mental disorders including Alzheimer's disease; anorexia nervosa; apathy; attention-deficit or other cognitive disorders due to general medical conditions including attention-deficit disorder (ADD) and attention-deficit hyperactivity disorder (ADHD) and it's recognized sub-types; reading disorders; bulimia nervosa; chronic fatigue syndrome; pain; chronic pain; cyclothymic disorder; depression including adolescent depression and minor depression; fibromyalgia and other somatoform disorders including somatization disorder; conversion disorder; pain disorder; hypochondriasis; body dysmorphic disorder; undifferentiated somatoform disorder; and somatoform NOS; incontinence including stress incontinence; genuine stress incontinence; and mixed incontinence; urinary disorders; premature ejaculation; inhalation disorders; intoxication disorders including alcohol addiction; mania; migraine headaches; obesity including reducing the weight of obese or overweight patients; restless legs syndrome; oppositional defiant disorder; peripheral neuropathy; diabetic neuropathy; post-herpetic neuralgic; premenstrual dysphoric disorder including premenstrual syndrome and late luteal phase dysphoric disorder; hot flashes; sleep disorders including narcolepsy, insomnia, enuresis, sleep walking disorder, and breathing related sleep disorder; specific developmental disorders; selective serotonin reuptake inhibition (SSRI) “poop out” syndrome; and TIC disorders including Tourette's Disease in a mammal, including a human, comprising administering to a mammal in need of such treatment an amount of a compound of the formula I as defined in claim 1.

8. A method according to claim 10, wherein the disorder or condition being treated is ADHD.

9. A method according to claim 10, wherein the disorder or condition being treated is fibromyalgia.

10. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression or reactive depression including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, including, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, including, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders including severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, including Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders including medication-induced movement disorders, including, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; pain; stress induced urinary incontinence; premature ejaculation; chemical dependencies and addictions including dependencies on, or addictions to, alcohol, heroin, cocaine, benzodiazepines, nicotine, or phenobarbitol and behavioral addictions such as an addiction to gambling; and ocular disorders such as glaucoma and ischemic retinopathy in a mammal, including a human, comprising administering to said mammal:
(a) a compound according to claim 1, or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.

11. A pharmaceutical composition comprising:
(a) a compound according to claim 1, or a pharmaceutically acceptable salt thereof;
(b) another pharmaceutically active agent that is an antidepressant or an anti-anxiety agent; and
a pharmaceutically acceptable carrier.

12. A method of treating attention deficit hyperactivity disorder (ADHD) comprising administering to a mammal in need of such treatment a therapeutically effective amount of a compound having the formula 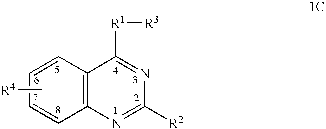
or a pharmaceutically acceptable salt, solvate or hydrate thereof, wherein
R¹ is (C₁-C₆)alkyl, (C₃-C₈)cycloalkyl, (C₁-C₆)alkoxy, aryl, amino, halogen, hydroxy, or a saturated, unsaturated, or aromatic five to seven membered monocyclic heterocyclic ring containing from one to three heteroatoms independently selected from oxygen, nitrogen, and sulfur;
R² is (C₁-C₆)alkyl, (C₃-C₈)cycloalkyl, or a saturated, unsaturated, or aromatic five to seven membered monocyclic heterocyclic ring or a six to ten membered bicyclic ring containing from one to three heteroatoms independently selected from oxygen, nitrogen, and sulfur, any of which may be unsubstituted or substituted with one or more of the following substituents: (C₁-C₆)alkyl, amino, (C₁-C₆)alkylamino, or a heterocyclic group;
R³ is independently selected from one or more of hydrogen, (C₁-C₆)alkyl, (C₃-C₈)cycloalkyl, amino, (C₁-C₆)alkylamino, or a heterocyclic group; and
R⁴ is a hydrogen, halogen, —NO₂, (C₁-C₆)alkyl, (C₁-C₆) alkoxy, or a heterocyclic group.

13. A compound having the formula 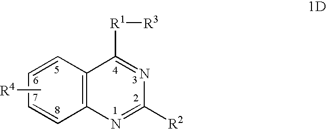
or a pharmaceutically acceptable salt, solvate, or hydrate thereof, wherein
R¹ is (C₁-C₆)alkyl, (C₃-C₈)cycloalkyl, (C₁-C₆)alkoxy; aryl, amino, halogen, hydroxy, or a saturated, unsaturated, or aromatic five to seven membered monocyclic heterocyclic ring containing from one to three heteroatoms independently selected from oxygen, nitrogen, and sulfur;
R² is piperidinyl unsubstituted or substituted with one or more of the following substituents: (C₁-C₆)alkyl, amino, (C₁-C₆)alkylamino, a heterocyclic group or a carboxylic acid or ester thereof;
R³ is independently selected from one or more of hydrogen, (C₁-C₆)alkyl, (C₃-C₈)cycloalkyl, (C₁-C₆)alkoxy, aryl, amino, halogen, or hydroxy groups; and
R⁴ is a hydrogen, halogen, —NO₂, (C₁-C₆)alkyl, (C₁-C₆) alkoxy, or a heterocyclic group, with the proviso that when R¹ is phenyl and R² is 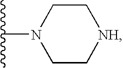
then R⁴cannot be halogen, methyl, or NO₂ at the 6-position.

14. A compound which inhibits norepinephrine reuptake and is 5-hydroxytryptamine-3 (5-HT3) antagonist.

15. A method of treating attention deficit hyperactivity disorder, schizophrenia, and age-related cognitive deficit, said method comprising the step of administering to a subject in need thereof a compound which is an inhibitor of norepinephrine reuptake and a 5-hydroxytryptamine(5-HT3) antagonist.
